SEQUENCE LISTING (22 sequences)

SEQ ID NO: 1 - DNA, 2520 bp, Thermobaculum terrenum
atgaaacatctggttaccacactgctgtttggtctggtttttgcagcagcagttcgtgcacagagcagccgtgttgatagcctgctggcagcaatgaccctggcagaaaaaatcagcctgctgcatggtgcaattgatccgaccggtgaagccggtgcaggttatgttccgggtgttccgcgtctgggtattccggcaatgcgtctgaccgatggtccggcaggtattcgtacccgtcatccggcaaccgcactgccagcaccggttgcactggcagccagctttgatccggaactggcatatcgttatggtcatgttatgggtattgaaggtcgtgcacgtcgtcatgaagttctgctgagcccgatggttaatattgttcgtgttccggaagcaggtcgtaactttgaaacctttagcgaagatccgctgctgagcgcagaaatggttgcagccgaagtgcgtggtattcaggatgcaggtatgatggcaaccgttaaacattatgtggccaacaactttgagaatgatcgtatgcgtgttaatgtggttgttgatgaacgtaccctgcgcgaaatttatctgcctggttttgaagcagccattaaagcgggtgcagcagccgttatgtgtgcatataatcgtgtgaatggtccgtatgcctgtgataatgaaatgctgctgaccgatattctgcgtgatgaatggggctttgaaggttgggttatgaccgattggtttgcaggtcatagcctggaaagcctggttcgtggtctggatcaagaaatgcctggttataccattccgtttagcagtccggatatgccgctggcaccggcagtttttgccgatagcctgttagccgcagttgaaagcggtcgtattgatgaagcctatgttgatcgtgcagttcgtcgtattctggttcagatggaacagtttggcctgctggatggcgaaagcacccctccggaaattaacattgaagcacatgcagccgttgcacgtgaagttgcagaagcaggcgctgttctgctgcgtaatgaaaatcagaccctgccgctgagcgaacgtgatctgcagcatctggttgttattggtccgaccgcaacacgtccgctgattggtggtggcggtagcagtcgtgttcagccgtttcgtaccacaagcactctggcagcactgcaagaactggcaggtccgcaggcacagattcgttatgtgccaggtattgatctggatggtatgccggttccgagcagcgcactgcgtacaccggatggtcagcctggtctgctgcgccagggtgcagatggtacaacccaggttgatgcacagctggattttaccggtgaacgtgcactgcctccgggtagccagtggacctggaccggtacactgaccgcaccgaccgcaggcgtttatgaactgaaactgcagacagccggtggtgttggcaccctgagcattgatggccagccggttctgcgtaccggtatgttttttagtgatgcaagcctgattccgacagcagatagtctggaaaatgcaacctatcgtattgaactgcaggcaggccagcagctgagcctgaccgttcagattagcggtcaggtgccgagcctgccgtttctgcctgccggtcagaatccggttcaggttcgtctggcatgggttacaccggaacgtcgtgcagcctttctggaagaagcagcggaagcagcacgtgcagcacatgccgcaattgtttttgtttatgaagagggcaccgaaggtcgcgatcgtgaaaccctggcactgcgtccggatcaggatgccctggttgaagcagttgccgcagcaaatccgcgtaccaccgttgttatgaatgttggtgcaccgacactgatgccgtgggcagaacgtgttggtgccattctgctgatgtggtatccgggtcaagaaggtggttgggccaccgcagatgtgctgctgggtcgtgcaaatccggcaggccgtctgccggttacctttccgcgtcgtgccgaagatgctccgaccgccagtccggaacgttatcctggtgttgatctgacagcacgttatgatgaaggcatttttgttggttatcgttggtatgatgcccagcagattgaaccgctgtttccgtttggtcatggtctgagctataccacctttgcctatgaaaatctgcgtgttgaacctgatggtgatggttttgttgttcgttttgtggtgcgtaataccggtgatcgcgcaggtagtgatgttccgcaggtttatctgggtccgcctgaaaatccgcctgttccgatggccgttcgtcagctggttggttttcgtcgtgttacactggcacctggtgaagcacaagaagttaccgttcgtatcgatggtcgtgccctgagctattggagcgttgaagatcatgcctgggttaaagcaaccggtcgtcgtaccctgtatgtgggtgcaagcgcacgcgatctgcgcctgcagaccgaaattgatgttcagtag

SEQ ID NO: 2 - DNA, 438 bp, Artificial Sequence
  Synthetic
  misc_feature (336)..(356): n is a, c, g, or t
attccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatcnnnnnnnnnnnnnnnnnnnnngttttagagctagaaatagcaagttaaaataaggctagtccgttatcaacttgaaaaagtggcaccgagtcggtgctttttt

SEQ ID NO: 3 - DNA, 71 bp, Artificial Sequence
  Synthetic
tgactgactctagaaataattttgtttaactttaagaaggagatataccatggacccgtatgaagatccgc

SEQ ID NO: 4 - DNA, 36 bp, Artificial Sequence
  Synthetic
gcatgcatggatccctacagggtcagaccatgaccg

SEQ ID NO: 5 - DNA, 69 bp, Artificial Sequence
  Synthetic
tgactgactctagaaataattttgtttaactttaagaaggagatataccatggcttcctccgaagacgt

SEQ ID NO: 6 - DNA, 34 bp, Artificial Sequence
  Synthetic
gcatgcatggatccttaagcaccggtggagtgac

SEQ ID NO: 7 - DNA, 74 bp, Artificial Sequence
  Synthetic
tgactgactctagaaataattttgtttaactttaagaaggagatataccatgaaacatctggttaccacactgc

SEQ ID NO: 8 - DNA, 40 bp, Artificial Sequence
  Synthetic
gcatgcatggatccctactgaacatcaatttcggtctgca

SEQ ID NO: 9 - DNA, 71 bp, Artificial Sequence; identical to SEQ ID NO: 3
  Synthetic

SEQ ID NO: 10 - DNA, 36 bp, Artificial Sequence; identical to SEQ ID NO: 4
  Synthetic

SEQ ID NO: 11 - DNA, 67 bp, Artificial Sequence
  Synthetic
tgactgactctagaaataattttgtttaactttaagaaggagatataccatgagcgtagcgcggttt

SEQ ID NO: 12 - DNA, 40 bp, Artificial Sequence
  Synthetic
gcatgcatggatccctaaccttccaccaaagcatttcttg

SEQ ID NO: 13 - DNA, 21 bp, Artificial Sequence
  Synthetic
actgcacgccgtaggtcaggg

SEQ ID NO: 14 - DNA, 21 bp, Artificial Sequence
  Synthetic
gcaactacaagacccgcgccg

SEQ ID NO: 15 - DNA, 21 bp, Artificial Sequence
  Synthetic
tcgatgcccttcagctcgatg

SEQ ID NO: 16 - DNA, 21 bp, Artificial Sequence
  Synthetic
tcaagatccgccacaacatcg

SEQ ID NO: 17 - DNA, 21 bp, Artificial Sequence
  Synthetic
ccatgccgagagtgatcccgg

SEQ ID NO: 18 - DNA, 21 bp, Artificial Sequence
  Synthetic
gccgcccgagagtcttaatcg

SEQ ID NO: 19 - DNA, 20 bp, Artificial Sequence
  Synthetic
gggcgaggagctgttcaccg

SEQ ID NO: 20 - DNA, 40 bp, Artificial Sequence
  Synthetic
atatatcgtctcgaattgaaagtatttcgatttcttgggt

SEQ ID NO: 21 - DNA, 82 bp, Artificial Sequence
  Synthetic
ataattcgtctctagcgcaaaacgcctaaccctaagcagattcttcatgcaattgtgtctagaaaaaagcaccgactcggtg

SEQ ID NO: 22 - DNA, 18 bp, Artificial Sequence
  Synthetic
atttaggtgacactatag

(19) United States
(12) Patent Application Publication
(10) Pub. No.: US 2020/0001302 A1
(43) Pub. Date: Jan. 2, 2020

(54) MICROFLUIDIC DEVICES, SYSTEMS, INFRASTRUCTURES, USES THEREOF AND METHODS FOR GENETIC ENGINEERING USING SAME

(71) Applicant: VALORBEC, SOCIÉTÉ EN COMMANDITE, Montréal (CA)

(72) Inventors: Steve SHIH, Montréal (CA); Mathieu HUSSER, Montréal (CA); Philippe VO, Brossard (CA); Hugo SINHA, Boston, MA (US)

(73) Assignee: VALORBEC, SOCIÉTÉ EN COMMANDITE, Montréal (CA)

(21) Appl. No.: 16/502,859

(22) Filed: Jul. 3, 2019

Related U.S. Application Data
(63) Continuation-in-part of application No. PCT/CA2018/051063, filed on Sep. 4, 2018.
(60) Provisional application No. 62/627,022, filed on Feb. 6, 2018.
(60) Provisional application No. 62/693,998, filed on Jul. 4, 2018.
(60) Provisional application No. 62/693,998, filed on Jul. 4, 2018.

(51) Int. Cl. B01L 3/00

(52) U.S. Cl. CPC B01L 3/502792; B01L 3/502715; B01L 3/50273; B01L 2200/06; B01L 2200/028; B01L 2400/0427; B01L 2200/027

(57) ABSTRACT

There are provided various microfluidics devices. Microfluidics devices that include a culture area for mixing a composition and an assay area for measuring enzyme activity of samples of the bacterial culture are, for example, provided. The assay area may include an optical density reader. The optical density reader may include a light emitting source and sensor to allow monitoring of the optical density of samples. Microfluidic devices comprising a first plate comprising at least one hydrophilic site are also provided as well as methods of manufacture thereof. Methods for performing analyses of compositions on microfluidics devices comprising a plate assembly having a first plate and a second plate are also provided in various embodiments.

### CROSS-REFERENCE TO RELATED APPLICATIONS

This application is a continuation-in-part of International patent application no. PCT/CA2018/051063, filed Sep. 4, 2018, which claims the benefit of priority from U.S. provisional application No. 62/627,022 filed on Feb. 6, 2018 and from U.S. provisional application No. 62/693,998 filed on Jul. 4, 2018. In addition, this application claims priority to said application No. 62/693,998. These documents are hereby incorporated by reference in their entirety.

### INCORPORATION OF SEQUENCE LISTING

A computer readable form of the Sequence Listing “P54910US02_SequenceListing.txt” (8.47 KB), submitted via EFS-WEB and created on Jun. 28, 2019, is herein incorporated by reference.

### FIELD OF THE DISCLOSURE

The present subject matter relates to systems and methods for controlling and manipulating droplets in a microfluidics device.

### BACKGROUND OF THE DISCLOSURE

Digital microfluidics (DMF) provides methods of manipulating nanoliter to microliter volumes of liquids on an array of electrodes. By applying an electric potential to an electrode, these discrete droplets can be controlled in parallel, transported, mixed, reacted, and analyzed. Typically, an automation system is interfaced with a DMF device that uses a standard set of basic instructions written by the user to execute droplet operations.

The integration of a capacitive feedback system with digital microfluidics use electronic circuits to sense and to monitor the droplet on device. However, a drawback with these methods is that these systems are not capable of detecting individual droplet failures. If a failure is detected, these systems require a re-application of a potential on the destination electrode for all the droplets on the device since it is not known which droplet on the device has failed in operation. This is not a favorable solution since excess activation of electrodes reduces the integrity of the dielectric and causes the surface to be prone to biofouling. Furthermore, these systems are only capable of sensing the droplet, but require external detectors (e.g., well-plate readers) for bioanalysis.

### SUMMARY

The present subject matter relates to systems and methods for controlling and manipulating droplets in a microfluidics device

According to one example, there is provided an image-based system for tracking droplet movement on a digital microfluidics device. The image-based system includes a computer vision system for capturing images of at least one droplet on one or more electrodes of the digital microfluidics device; a control unit configured to manipulate the at least one droplet on the one or more electrodes of the digital microfluidics device; and an interface unit electrically coupled to the computer vision system and electrically coupled to the control unit. The interface unit is configured to: direct the control unit to manipulate the at least one droplet on the one or more electrodes of the digital microfluidics device; receive images of the at least one droplet on the one or more electrodes of the digital microfluidics device, the images captured by the computer vision system; and determine, based on the images captured by the computer visions system, a position of the at least one droplet on the one or more electrodes of the digital microfluidics device.

According to one example, there is provided a microfluidics device including: an optical density (OD) reader, wherein the optical density reader comprises a light emitting source and sensor to allow monitoring of the optical density of samples of a bacterial culture cultivated in the device.

According to one example, there is provided a microfluidics device including: a culture area for mixing bacterial culture; and an assay area for measuring enzyme activity of samples of the bacterial culture, the assay area comprising an optical density reader, wherein the optical density reader comprises a light emitting source and sensor to allow monitoring of the optical density of samples of the bacterial culture.

According to one example, there is provided a microfluidics device including: a culture area for mixing bacterial culture; at least one reservoir for storing reagents for inducing the bacterial culture; a waste area for discharging waste of the bacterial culture; and an assay area for measuring enzyme activity of samples of the bacterial culture, the assay area comprising an optical density reader, wherein the optical density reader comprises a light emitting source and sensor to allow monitoring of the optical density of samples of the bacterial culture.

According to one example, there is provided a method of inducing bacterial culture in a microfluidics system, including: inducing bacterial culture; carrying out at least one incubation of the bacterial culture in a micro-array; quenching the incubated bacterial culture; and reading optical density of samples of the quenched bacterial culture.

According to one example, there is provided a method of inducing bacterial culture in a microfluidics system, including: inducing the bacterial culture; carrying out two incubations of the bacterial culture in a micro-array, wherein the two incubations are carried at different times; quenching the incubated bacterial culture; and reading optical density of samples of the quenched bacterial culture.

According to one example, there is provided an image-based system for automating and tracking droplet movement on a digital microfluidics device, including: a computer vision system for acquiring images used to detect droplets on the digital microfluidics device; a control unit for manipulating droplets in a digital microfluidics device; and a graphical user interface for programming droplet operations, tracking droplet movements and visualizing current droplet manipulations.

According to one example, there is provided a method for operation an AIMS comprising: inserting the device into the OD reader; loading the reagents onto the device; and inputting a series of desired droplet movement steps such that induction (and cell culture and analysis) is performed by the AIMS.

According to one example, there is provided a method for operating an image-based feedback system, comprising: resting a droplet a first electrode; applying a potential to a second electrode; capturing a frame after actuation; creating a difference frame by taking the difference from a grayscale image and a reference image (e.g. no dispensed droplets); creating a binarized frame from the difference frame; detecting circles from this frame through a Hough transform; and returning a successful or unsuccessful result depending on the location of the actuated droplet and the user-defined detection box.

According to one example, there is provided a method for operating a digital microfluidic device, comprising: moving a droplet in the digital microfluidic device to take an optical density (OD) reading of the droplet.

According to one example, there is provided a method for building a digital microfluidics (DMF) device comprising: drawing a design of the DMF device; printing a photomask of the DMF device; forming a bottom plate and a top-plate, wherein the bottom plate and top plate are formed of substrates; imprinting transparency mask designs chromium substrates to form the bottom plate, such the substrates are coated with photoresist material; rinsing the coated substrates and drying them under a gas stream and baking them; etching the exposed chromium of the substrates, rinsing the substrates and drying it under a gas stream; and assembling the device by joining the top-plate to the bottom plate.

According to one example, there is provided a microfluidic device comprising: a first plate comprising at least one hydrophilic site.

According to one example, there is provided a microfluidic device comprising: a plate assembly comprising a first plate and a second plate that are separated from one another by a separation material; wherein the first plate comprises at least one hydrophilic site.

According to one example, there is provided a method for performing an analysis of a composition on a microfluidics device comprising a plate assembly having a first plate and a second plate, the method comprising: dispensing a composition on the second plate of the microfluidics device; conveying the composition from the second plate to first plate by using gravity, such that the composition transferred from the second plate to the first plate; and analyzing or treating the composition on the first plate.

According to one example, a microfluidic device is provided. The microfluidic device includes: a first plate including: a cell culture region for maintaining a cell culture; an optical density reader for measuring an optical density of at least a portion of the cell culture; a hydrophilic site between the cell culture region and the optical density reader, the hydrophilic site for presenting the at least a portion of the cell culture to the optical density reader; and a second plate comprising electrodes that, when actuated, control movement of the at least a portion of the cell culture to the hydrophilic site to be measured by the optical density reader.

According to one example, a microfluidic device is provided. The microfluidic device includes a first plate comprising: a cell culture region for maintaining a cell culture; a reservoir for storing reagents to induce at least a portion of the cell culture; and a hydrophilic site between the cell culture region and the reservoir for mixing the at least a portion of the cell culture and at least a portion the reagents to induce the at least a portion of the cell culture; and a second plate spaced apart from the first plate, the second plate comprising electrodes that, when actuated, control movement of the at least a portion of the cell culture and the at least a portion of the reagents to the hydrophilic site.

According to one example, there is provided method of inducing protein expression by cells in a cell culture on a microfluidic device. The microfluidic device includes a plate assembly having a first plate and a second plate. The method includes monitoring an optical density of at least a portion of the cell culture; when the optical density of the at least a portion of the composition reaches a threshold optical density, moving the at least a portion of the cell culture to a hydrophilic site of the microfluidic device; and combining an inducing agent with the at least a portion of the cell culture at the hydrophilic site of the microfluidic device to induce protein expression by the cells in the cell culture at the hydrophilic site of the microfluidic device. The moving of the at least a portion of the cell culture to the hydrophobic site includes sequentially actuating electrodes of the second plate to control movement of the at least a portion of the cell culture to the hydrophilic site.

In one aspect, the present invention is generally directed to a method of gene-editing a cell, such as a mammalian cell. In one set of embodiments, the method comprises culturing mammalian cells within a digital microfluidics device, dispensing a droplet containing a transfection complex on the digital microfluidics device; and exposing the mammalian cells to the droplet to transfect at least some of the mammalian cells using the transfection complex.

In another set of embodiments, the method includes culturing mammalian cells within a digital microfluidics device, transfecting the mammalian cells within the digital microfluidics device, and determining transfection of the mammalian cells within the digital microfluidics device.

The method, in still another set of embodiments, comprises culturing mammalian cells within a digital microfluidics device, and transfecting the mammalian cells within the digital microfluidics device.

The present invention, in another aspect, is directed to a digital microfluidics device. In accordance with one set of embodiments, the device comprises a first reservoir containing a fluid containing plasmid, a second reservoir containing a fluid containing a transfection reagent, a mixing region fluidly connected to the first reservoir and the second reservoir, where the mixing region contains a droplet containing a transfection complex formed from the plasmid and the transfection reagent, a media reservoir containing cell culture media, and a plurality of cell culture regions, each fluidly connected to the mixing region and the media reservoir. In some cases, the plurality of cell culture regions comprises mammalian cells.

The below-presented examples are non-limitative and are used to better exemplify various embodiments of the present disclosure, including but not limited to any of the embodiments described above.

For example, the microfluidics device further includes an absorbance reading electrode, the absorbance reading electrode comprising a transparent section, such that the optical density reader measures a sample of the composition deposited on the absorbance reading electrode.

For example, the transparent section is in the middle, center, or edge of the absorbance reading electrode.

For example, the light emitting source is placed above the absorbance reading electrode and the sensor is placed on the absorbance reading electrode for monitoring of the optical density of samples of the bacterial culture.

For example, the light emitting source is placed above the transparent window of the absorbance reading electrode and the sensor is placed below the transparent window for reading intensity of the light passing emitted by the light emitting source.

For example, the absorbance reading electrode comprises a width of about 2.25 mm and a length of about 2.25 mm.

For example, the transparent section comprises a width of about 0.75 mm and a length of about 0.75 mm.

For example, the light emitting source comprises a 600 nm light emitting source.

For example, the sensor is a photodiode sensor.

For example, the method of inducing a composition in a microfluidics system further includes monitoring the optical density of the composition to induce it at an optimal value.

For example, the method further includes monitoring the optical density of the composition to induce it at a desired time.

For example, the computer vision system detects a size of the at least one droplet and/or singular droplet dispensing and movement failures on the digital microfluidics device.

For example, the control unit senses the at least one droplet on an electrode of the digital microfluidics device.

For example, the control unit controls the at least one droplet on an electrode of the digital microfluidics device by applying a potential to the electrode.

For example, the control unit senses the at least one droplet on the electrode and reapplies the potential at the electrode if the droplet is not present on that electrode.

For example, a user can provide, through the interface, a set of instructions to the control unit for dispensing, moving, splitting and mixing the droplets on the digital microfluidics device.

For example, a user, through the interface, builds a grid corresponding to a device grid of the digital microfluidics device.

For example, a user, through the interface, generates a sequence of droplet operations on the grid.

For example, a user, through the interface, imports the sequence of droplet operations to the digital microfluidics device, such that the interface provides a set of instructions to the controls unit for executing a same sequence of droplet operations on the device grid of the digital microfluidics device.

For example, the computer vision system monitors the same sequence of droplet operations on the device grid of the digital microfluidics device and provides feedback to the interface.

For example, the feedback comprises at least one of image data and/or video data.

For example, the interface is a graphical user interface.

For example, the control unit detects whether the at least one droplet is located at a destination electrode by: instructing the computer vision to capture a frame of the position of the at least one droplet on an electrode source; determining a difference image by subtracting a reference image from the frame to identify a boundary of the at least one droplet; and detecting whether the at least one droplet is on the destination electrode on the difference image.

For example, if the at least one droplet is not detected on the destination electrode, the control unit initiates a feedback process by actuating the source electrode of the at least one droplet; actuating the destination electrode of the at least one droplet; pausing for a predetermined amount of time; turning off the source electrode; incrementing the voltage at the electrode by a predetermined voltage amount; and turning off the destination electrode.

For example, the control unit detects whether the at least one droplet is located at a destination.

For example, the method further includes adding an inducer to the droplet in the digital microfluidic device.

For example, the method further includes incubating the droplet in the digital microfluidic device.

For example, the method further includes immersing the substrates in a silane composition for dielectric priming; and optionally rinsing the substrates and drying under a gas stream.

For example, the method further includes adding polymer coatings to the substrates.

For example, the method further includes depositing a dielectric coating on the substrates; and optionally coating the substrates with a hydrophobic coating.

For example, the top plate comprises a ground electrode formed from an indium tin oxide (ITO) or any metal-coated substrate.

For example, the method further includes spin-coating FluoroPel or hydrophobic-based coating on the indium-tin oxide.

For example, the ITOs is cleaned by immersion in an RCA solution comprising of DI water, aqueous ammonium hydroxide, and hydrogen peroxide.

For example, after rinsing, drying, and dehydrating, the substrates are spin-coated with photoresist; and optionally baked.

For example, the substrates are exposed through the photomask with an array of six 1.75 mm diameter circular features; and optionally, after rinsing, air-drying and dehydrating, the top-plate is then flood exposed, spin-coated with Teflon, and post-baked.

For example, after being allowed to cool, the substrates are immersed in acetone with agitation until the Teflon-AF over patterned sites is lifted off; optionally, after being rinsed with DI water and dried under a stream of nitrogen, droplets of AZ300T stripper are placed over the spots and the substrates are placed aside followed by rinsing with DI water and air-drying; and optionally post-baking followed to reflow the Teflon-AF

For example, the substrates comprises glass, paper, silicon, or semiconductor-based elements.

For example, the first plate comprises an electrode layer supported by an electrically insulating substrate.

For example, the electrode is formed from an indium tin oxide (ITO) or any metal-coated glass substrate.

For example, the first plate is a top plate.

For example, the first plate is detachable.

For example, at least one hydrophilic site is configured for dispensing a composition for culture.

For example, at least one hydrophilic site is fabricated with an electrode and used for cell sensing.

For example, the first plate comprises an electrode formed from an indium tin oxide (ITO) coated glass substrate.

For example, the top plate is used to culture cells on the hydrophilic spots.

For example, the top plate is used to integrate other electrodes for transformation or transfection experiments on the microfluidic device.

For example, the first plate is used to exchange of reagents on the microfluidic device.

For example, the first plate can hold magnetic beads while exchanging liquid on the microfluidic device.

For example, the first plate is a top-plate and the second plate is a bottom plate.

For example, the first plate comprises at least six hydrophilic sites.

For example, at least one hydrophilic site comprises a diameter of about 1.5 mm.

For example, at least one hydrophilic site comprises a diameter of about 1 mm to about 2 mm.

For example, at least one hydrophilic site comprises a diameter of about 0.1 mm to about 5 mm.

For example, the second plate comprises electrodes for manipulating droplets and the electrodes comprise dielectric and/or hydrophobic layers.

For example, the electrodes of the second plate are metal-patterned.

For example, the second plate comprises electrodes formed on an electrically insulating substrate, the electrode being coated with a dielectric layer having a hydrophobic surface.

For example, the separation material is a spacer of about 5 micrometers to about 240 micrometers.

For example, the separation material is a spacer of about 100 micrometers to about 180 micrometers.

For example, the separation material is a spacer of about 130 micrometers to about 150 micrometers.

For example, the separation material comprises a dielectric spacer to form an inner channel for supporting and transporting droplets and/or delivering fluids to refill reservoirs.

For example, treating the composition comprises one of: mixing the composition with another substance, diluting the composition, incubating the composition, culturing the composition, performing knock out experiments on the composition and performing transfection experiments on the composition.

For example, the method further includes analyzing or treating the composition on a hydrophilic site of the first plate.

For example, the method further includes monitoring the composition on the microfluidics device.

For example, monitoring the composition on the microfluidics device is performed by microscopy.

For example, monitoring the composition on the microfluidics device is performed by taking images of the composition and analyzing the images on a computing device.

For example, analyzing the images comprising at least one of: image cropping, identifying individual and overlapping cells in the composition, counting total number of cells, measuring the size and shape of the cells, creating binary images of the cells, and comparing knocked-out and non-knocked out cells.

For example, the method can be used for gene editing and analysis.

For example, the composition comprises a bacterial culture and/or a gene.

For example, the method can be carried out by using the microfluidic device described herein.

For example, the method includes conducting a gene-editing assay with the microfluidic device described herein.

For example, the method of using the device includes conducting gene transfection and/or knockout procedures.

For example, the method of using the device includes editing cancer cells with said device.

For example, the device can further comprises an absorbance reading electrode, the absorbance reading electrode comprising a transparent section, such that the optical density reader measures a sample of the composition deposited on the absorbance reading electrode.

For example, the transparent section is in the middle, center, or edge of the absorbance reading electrode.

For example, the light emitting source can be placed above the absorbance reading electrode and the sensor is placed on the absorbance reading electrode for monitoring of the optical density of samples of the bacterial culture.

For example, the light emitting source can be placed above the transparent window of the absorbance reading electrode and the sensor is placed below the transparent window for reading intensity of the light passing emitted by the light emitting source.

For example, the absorbance reading electrode can comprise a width of about 1 to about 3 mm and a length of about 1 to about 3 mm.

For example, the absorbance reading electrode can comprise a width of about 2.25 mm and a length of about 2.25 mm.

For example, the transparent section can comprise a width of about 0.5 to about 1.5 mm and a length of about 0.5 to about 1.5 mm.

For example, the transparent section can comprise a width of about 0.75 mm and a length of about 0.75 mm.

For example, the light emitting source can comprise a 600 nm light emitting source.

For example, the light emitting source can comprise a 500 to 700 nm light emitting source.

For example, the sensor can be a photodiode sensor.

For example, the method can further comprise monitoring the optical density of the composition to induce it at an optimal value.

For example, the method can further comprise monitoring the optical density of the composition to induce it at a desired time.

For example, the computer vision system can detect a size of the at least one droplet and/or singular droplet dispensing and movement failures on the digital microfluidics device.

For example, the control unit can sense the at least one droplet on an electrode of the digital microfluidics device.

For example, the control unit can control the at least one droplet on an electrode of the digital microfluidics device by applying a potential to the electrode.

For example, the control unit can sense the at least one droplet on the electrode and reapplies the potential at the electrode if the droplet is not present on that electrode.

For example, a user can provide, through the interface, a set of instructions to the control unit for dispensing, moving, splitting and mixing the droplets on the digital microfluidics device.

For example, a user, through the interface, can build a grid corresponding to a device grid of the digital microfluidics device.

For example, a user, through the interface, can generate a sequence of droplet operations on the grid.

For example, a user, through the interface, can import the sequence of droplet operations to the digital microfluidics device, such that the interface provides a set of instructions to the controls unit for executing a same sequence of droplet operations on the device grid of the digital microfluidics device.

For example, the computer vision system can monitor the same sequence of droplet operations on the device grid of the digital microfluidics device and provides feedback to the interface.

For example, the feedback can comprise at least one of image data and/or video data.

For example, the interface can be a graphical user interface.

For example, the control unit can detect whether the at least one droplet is located at a destination electrode by: instructing the computer vision to capture a frame of the position of the at least one droplet on an electrode source; determining a difference image by subtracting a reference image from the frame to identify a boundary of the at least one droplet; and detecting whether the at least one droplet is on the destination electrode on the difference image.

For example, if the at least one droplet is not detected on the destination electrode, the control unit can initiate a feedback process by: actuating the source electrode of the at least one droplet; actuating the destination electrode of the at least one droplet; pausing for a predetermined amount of time; turning off the source electrode; incrementing the voltage at the electrode by a predetermined voltage amount; and turning off the destination electrode.

For example, the control unit can detect whether the at least one droplet is located at a destination.

For example, the method can further comprise adding an inducer to the droplet in the digital microfluidic device For example, the method can further comprise incubating the droplet in the digital microfluidic device.

For example, the method can further comprise immersing the substrates in a silane composition for dielectric priming; and optionally rinsing the substrates and drying under a gas stream.

For example, the method can further comprise adding polymer coatings to the substrates.

For example, the method can further comprise depositing a dielectric coating on the substrates; and optionally coating the substrates with a hydrophobic coating.

For example, the top plate can comprise a ground electrode formed from an indium tin oxide (ITO) or any metal-coated substrate.

For example, the method can further comprise spin-coating FluoroPel or hydrophobic-based coating on the indium-tin oxide.

For example, the ITOs can be cleaned by immersion in an RCA solution comprising DI water, aqueous ammonium hydroxide, and hydrogen peroxide.

For example, after rinsing, drying, and dehydrating, the substrates can be spin-coated with photoresist; and optionally baked.

For example, the substrates can be exposed through the photomask with an array of six 1.75 mm diameter circular features; and optionally, after rinsing, air-drying, and dehydrating, the top-plate can be flood exposed, spin-coated with Teflon, and post-baked.

For example, after being allowed to cool, the substrates can be immersed in acetone with agitation until the Teflon-AF over patterned sites is lifted off; optionally, after being rinsed with DI water and dried under a stream of nitrogen, droplets of AZ300T stripper are placed over the spots and the substrates are placed aside followed by rinsing with DI water and air-drying; and optionally post-baking followed to reflow the Teflon-AF.

For example, the substrates can comprise glass, paper, silicon, or semiconductor-based elements.

For example, the first plate can comprise an electrode layer supported by an electrically insulating substrate.

For example, the electrode can be formed from an indium tin oxide (ITO) or any metal-coated glass substrate.

For example, the first plate can be a top plate.

For example, the first plate can be detachable.

For example, the at least one hydrophilic site can be configured for dispensing a composition for culture.

For example, the at least one hydrophilic site can be fabricated with an electrode and used for cell sensing.

For example, the first plate can comprise an electrode formed from an indium tin oxide (ITO) coated glass substrate.

For example, the top plate can be used to culture cells on the hydrophilic spots.

For example, the top plate can be used to integrate other electrodes for transformation or transfection experiments on the microfluidic device.

For example, the first plate can be used to exchange of reagents on the microfluidic device.

For example, the first plate can hold magnetic beads while exchanging liquid on the microfluidic device.

For example, the first plate can be a top-plate and the second plate can be a bottom plate.

For example, the first plate can comprise at least six hydrophilic sites.

For example, the at least one hydrophilic site can comprise a diameter of about 1.5 mm.

For example, the at least one hydrophilic site can comprise a diameter of about 1 mm to about 2 mm.

For example, the at least one hydrophilic site can comprise a diameter of about 0.1 mm to about 5 mm.

For example, the second plate can comprise electrodes for manipulating droplets and wherein the electrodes comprise dielectric and/or hydrophobic layers.

For example, the second plate can comprise electrodes formed on an electrically insulating substrate, the electrode being coated with a dielectric layer having a hydrophobic surface.

For example, the separation material can be a spacer of about 5 micrometers to about 240 micrometers.

For example, the separation material can be a spacer of about 100 micrometers to about 180 micrometers.

For example, the separation material can be a spacer of about 130 micrometers to about 150 micrometers.

For example, the separation material can comprise a dielectric spacer to form an inner channel for supporting and transporting droplets and/or delivering fluids to refill reservoirs.

For example, treating the composition can comprise one of: mixing the composition with another substance, diluting the composition, incubating the composition, culturing the composition, performing knock out experiments on the composition and performing transfection experiments on the composition.

For example, the method can further comprise analyzing or treating the composition on a hydrophilic site of the first plate.

For example, the method can further comprise monitoring the composition on the microfluidics device.

For example, monitoring the composition on the microfluidics device can be performed by microscopy.

For example, monitoring the composition on the microfluidics device can be performed by taking images of the composition and analyzing the images on a computing device.

For example, analyzing the images can comprise at least one of: image cropping, identifying individual and overlapping cells in the composition, counting total number of cells, measuring the size and shape of the cells, creating binary images of the cells, and comparing knocked-out and non-knocked out cells.

For example, the methods described above can be used for gene editing and analysis.

For example, the composition can comprise a bacterial culture and/or a gene.

For example, the methods described above can be carried out by using the microfluidic device.

For example, there is provided a method of using a device of the disclosure, comprising conducting a gene-editing assay with said device.

For example, there is provided a method of using a device of the disclosure, comprising conducting gene transfection and/or knockout procedures.

For example, there is provided a method of using a device of the disclosure, comprising editing cancer cells with said device.

For example, there is provided the use of a device of the disclosure, for gene editing and/or analysis.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. The following drawings are presented as non-limitative examples:

FIG. 1 is a schematic of an image-based DMF feedback system, according to one example.

FIG. 2 illustrates fabrication of a 3D enclosure for an Automated Induction Microfluidic System (AIMS), according to one example.

FIG. 3 illustrates a circuit diagram showing the connectivity of one output that connects to a pogo pin, according to one example.

FIG. 4A and FIG. 4B illustrate schematics showing the actuation schemes tested with the imaging feedback system, according to one example.

FIG. 5 illustrates devices including different sized electrodes, according to one example.

FIG. 6 illustrates a plasmid map of pET_BGL1 having a pET16b backbone with BGL1, according to one example.

FIG. 7 illustrates a sequence (SEQ ID NO: 1) of β-glucosidase (BGL) from Thermobaculum terrenum.

FIG. 8 illustrates an algorithm of the image-based feedback system, according to one example.

FIG. 9 is a flowchart summarizing the algorithm used to manage the image-based feedback system, according to one example.

FIG. 10A shows a setup of a camera with the measured angle surrounded with a white backdrop.

FIG. 10B illustrates a set of images showing the success of droplet detection as a function of camera angle (°) at different light intensities (lux).

FIG. 11 illustrates the effect of electrode dimension and droplet radius on droplet detection, according to one example.

FIG. 12 illustrates multiplexed dispensing showing detection of a single droplet dispensing failure, according to one example.

FIG. 13 illustrates the effect of droplet movement on DMF devices without feedback, according to one example.

FIG. 14 illustrates a chemical scheme of the enzymatic assay.

FIG. 15 illustrates a curve depicting the average blue channel pixel intensity as a function of time.

FIG. 16 illustrates off-chip enzymatic assay with an absorbance readout as a function of time were collected every 30 min, according to one example.

FIG. 17 illustrates the layout of an AIMS device, according to one example.

FIG. 18 illustrates a comparison of bacterial growth on the AIMS with a macro-scale culture, according to one example.

FIG. 19 illustrates an automated induction using the AIMS, according to one example.

FIG. 20A and FIG. 20B illustrate an automation system for DMF, according to one example.

FIG. 21A illustrates images from a movie of an AIMS showing the step of automated culture, induction and protein analysis, according to one example.

FIG. 21B illustrates comparison of dose-response curves of isopropyl beta-D-1-thiogalactopyranoside (IPTG) using AIMS and macroscale cultures, according to one example.

FIG. 21C illustrates comparison of the rates of activity for three enzymes relative to the lowest (BGL1), according to one example.

FIG. 21D illustrates induction profile of the highest activity enzyme over 6 h on the AIMS, according to one example.

FIG. 22A illustrates a simulated output of a circuit, according to one example.

FIG. 22B illustrates a schematic showing the online integration of fluorescence detecting with the AIMS, according to one example.

FIG. 23A illustrates a side view of a thin film transistor (TFT)-DMF device, according to one example.

FIG. 23B illustrates an image of the fabricated TFT-DMF device, according to one example.

FIG. 23C illustrates a measured I-V curve of 3×3 transistors, according to one example.

FIG. 23D illustrates a schematic of the TFT devices used for factorial experiments, according to one example.

FIG. 24 illustrates gel electrophoresis of the polymerase chain reaction (PCR) products derived from amplification of the pET16b vector containing the synthetic inserts red fluorescent protein (RFP), BGL1, BGL2 and BGL3, according to one example.

FIG. 25 is a schematic of the plasmid, according to one example.

FIG. 26 is a growth curve for BL21 E. coli cultured under normal culturing conditions with and without 0.05% Pluronics F-68, according to one example.

FIG. 27 illustrates expression optimization assay to discover highly active BGL conducted in well-plates, according to one example.

FIG. 28A illustrates the relationships between a function generator and amplifier, a control board, Arduino Uno, a pogo pin board and an optical density (OD) reader with DMF device, according to one example.

FIG. 28B illustrates the relationships between a function generator and amplifier, a control board, Arduino Uno, a pogo pin board and an OD reader with DMF device, according to one example.

FIG. 28C illustrates a schematic of a DMF device, according to one example.

FIG. 28D illustrates a schematic of a DMF device, according to one example.

FIG. 29 illustrates a sequence of droplet operation using AIMS, according to one example.

FIG. 30A illustrates a sequence of droplet operation using AIMS, according to one example.

FIG. 30B illustrates a comparison of the conventional and microfluidic induction protocol, according to one example.

FIG. 31A to FIG. 31D illustrate characterization of the AIMS, according to examples.

FIG. 32A to FIG. 32C illustrate inducer concentration optimization, according to one example.

FIG. 33A to FIG. 33D illustrate expression optimization (single- and multi-point) assay to discover highly active BGL, according to one example.

FIG. 34 illustrates a top-view schematic of a digital microfluidic device, according to one example.

FIG. 35 illustrates a view schematic showing adherent cells culture on a top-plate, according to one example.

FIG. 36 illustrates a step-by-step CRISPR-Cas9 knock-out process at the cellular level, according to one example.

FIG. 37A illustrates a schematic showing the imaging pipeline used for analyzing transfection, according to one example.

FIG. 37B illustrates microscopy images of mCherry-transfected NCI-H1299 cells in a well-plate format and on a DMF device, according to one example.

FIG. 37C illustrates a video sequence depicting the mixing of lipids and DNA and the passive dispensing procedure onto the hydrophilic spot, according to one example.

FIG. 37D illustrates a plot showing the optimization of the lipid complex to media ratio for transfection on a device, according to one example.

FIG. 37E illustrates a plot of the transfection efficiency for a mCherry plasmid in the well-plate and on DMF devices, according to one example.

FIG. 38A illustrates a schematic showing the imaging pipeline used for analyzing knockout, according to one example.

FIG. 38B illustrates an image set (Hoechst, GFP, overlap) processed by CellProfiler to assess eGFP knock-out efficiency, according to one example.

FIG. 38C illustrates a plasmid map of the pCRISPR plasmid used showing the transgene integration in NCI-H1299 and sgRNA target regions of eGFP, according to one example.

FIG. 38D illustrates a plot for the knockout of GFP in well-plates compared to the microscale, according to one example.

FIG. 39A illustrates a signal transduction in the Ras pathway that leads to eventual cell proliferation, according to one example.

FIG. 39B illustrates microscopy images of the H1299 cells with sorafenib inhibitor (0 and 120 micromolar in DMSO) and with guide targeting RAF1 and eGFP (control), according to one example.

FIGS. 39C and 39D illustrate (FIG. 39C) on-chip and (FIG. 39D) off-chip dose-response curve for H1299 cells transfected with and without individual guides targeting Raf-1 at different concentrations of sorafenib, according to one example.

FIG. 40 illustrates the sgRNA sequence (SEQ ID NO: 2) representing the template designed for all sgRNAs, according to one example.

FIG. 41 illustrates a gel electrophoresis image of the PCR products of the synthesized CRISPR guides, yielding g-blocks, according to one example.

FIG. 42 illustrates a schematic showing the procedure of inserting a CRISPR guide into a Cas9 vector backbone, according to one example.

FIG. 43 is a schematic of DMF device and top-plate fabrication, according to one example.

FIG. 44 illustrates a microfluidic automation system, according to one example.

FIG. 45A illustrates a cell humidified chamber with cover to prevent evaporation of droplets, according to one example.

FIG. 45B illustrates a microscope holder tailored to digital microfluidic devices, with opaque cover for fluorescence microscopy, according to one example.

FIG. 46A illustrates an optimization of chip configuration and electrode design with square electrodes, according to one example.

FIG. 46B illustrates interdigitated electrodes to facilitate droplet movement, according to one example.

FIG. 47 illustrates an optimization of on-chip transfection using various dilutions of lipid complexes in liquid media, according to one example.

FIG. 48 illustrates a western Blot showing Cas9 protein levels comparing different starting material of Cas9 into NCI-H1299 cells, according to one example.

FIG. 49 illustrates a plot of the transfection efficiency for both the All_in_one_CRISPR/Cas9 LacZ (pCRISPR) and mCherry2-N1, according to one example.

FIG. 50 illustrates a plot showing progression of cell viability over time, according to one example.

FIG. 51 illustrates microscopy images of H1299 cells on-chip, according to one example.

FIG. 52 illustrates raw data showing the absolute fluorescence and the morphology of the H1299 cells, according to one example.

### DETAILED DESCRIPTION

There are provided various microfluidics devices. Microfluidics devices that include a culture area for mixing a composition and an assay area for measuring enzyme activity of samples of the bacterial culture are, for example, provided. The assay area may include an optical density reader. The optical density reader may include a light emitting source and sensor to allow monitoring of the optical density of samples. Microfluidic devices comprising a first plate comprising at least one hydrophilic site are also provided as well as methods of manufacture thereof. Methods for performing analyses of compositions on microfluidics devices comprising a plate assembly having a first plate and a second plate are also provided in various embodiments.

In addition, various aspects of the present invention are generally directed to systems and methods for transfecting cells, including mammalian and bacterial cells, within a device such as a digital microfluidics device (DMF), e.g., on a suitable surface within the DMF. Examples of cells that can be transfected include bacterial cells such as E. coli, or mammalian cells such as human cells. However, other cells can be transfected as well, for example, other single-cell organisms, prokaryotic cells, eukaryotic cells, plant cells, or animal cells, including non-human cells, for example, an invertebrate cell, a fish cell, an amphibian cell, a reptile cell, a bird cell, or a non-human mammal, such as a monkey, cow, sheep, goat, horse, rabbit, pig, mouse, rat, dog, or cat. If the cell is from a multicellular organism, the cell may be from any part of the organism. In some cases, the cell may be a genetically engineered cell; in other cases, the cell is not genetically engineered. The cell may be an isolated cell, a cell aggregate, an organoid, in a tissue construct, or the like. One or more than one cell may be present. If more than one cell is present, the cells may be of the same or different types. In some embodiments, the cells are stem cells. In addition, in some cases, the cells are cancerous. Non-limiting examples of cancer cells include lung cancer cells, bladder cancer cells, brain cancer cells, breast cancer cells, liver cancer cells, prostate cancer cells, stomach cancer cells, leukemia cells, etc.

As discussed herein, in one set of embodiments, the cells may be cultured within a device such as a digital microfluidics device (DMF), e.g., prior to transfection within the device. Those of ordinary skill in the art will be familiar with techniques for culturing cells within a device. For example, culturing may be performed such that one or more conditions such as temperature, pressure, relatively humidity, lighting, sterility, concentrations of gases such as O₂, CO₂ (e.g., at 5 vol %), etc., are controlled. For instance, mammalian cells may be cultured at conditions of about 95% relative humidity and/or about 37° C. Other temperatures and/or relative humidities may be used as well in other embodiments. In some cases, a device such as is discussed herein may be partially or fully contained within an incubator or a humidified chamber, for example, to control such conditions. For example, in certain embodiments, the humidity may be kept at saturation (100% relatively humidity) to minimize evaporation from fluids within the device, such as from droplets containing cells. In some cases, the incubator may be obtained commercially.

In addition, in some cases, the temperature of at least a portion of the surface of the DMF device may be controlled, e.g., to provide temperatures suitable for culturing cells. For example, at least a portion of the surface, such as a cell culture region or an incubation region, may be controlled to produce a suitable temperature, e.g., a temperature of about 37° C., about 32° C., or the like. In some cases, the entire surface may be controlled to produce a suitable temperature. Those of ordinary skill in the art will be aware of suitable techniques to control the temperature of a surface or portion thereof, e.g., using temperature sensors, heat sources (e.g., electrically resistive heat sources), controllers such as PID controllers, and the like.

In addition, in certain embodiments, fluids may be delivered to and/or from the cell culture regions, e.g., using droplet manipulation techniques such as those discussed herein. For example, fluids such as cell culture media, transfection reagents, etc. may be delivered to the cell culture region, and/or fluids such as waste fluids (e.g., containing cell by-products) may be removed from the cell culture regions, using such techniques.

For example, in certain embodiments, cells may be cultured in any suitable media. Some or all of the cell culture media may be brought to a cell culture region within the DMF device, or externally from the DMF device. The media may be brought to the cells at any suitable time, which may be fixed (e.g., once per day), and/or when conditions of the cell culture indicate more media is necessary, e.g., due to changes in pH, optical density, opacity, color, or the like. The media may be brought to the cell culture region, for example, from a reservoir or a media feed region, e.g., using droplet manipulation techniques such as those discussed herein. The reservoir or a media feed region may be contained within the DMF device, for instance, on a surface within the DMF device used for culturing cells, or from a separate location, which may be a reservoir contained within the DMF device (e.g., within a bottle or other container), or separate from the DMF device, etc. (for example, pumped or otherwise delivered into the DMF device, e.g., as needed) Those of ordinary skill in the art will be familiar with a variety of cell culture media, such as DMEM, MEM, RPMI 1640, FBS, Ham's F-10, LB medium, or the like.

In some cases, the cell culture media may be present as a droplet surrounding the cells and/or a cell culture region. The droplet may have any suitable size or volume. For example, the droplet may have a volume of at least 10 microliters, at least 30 microliters, at least 50 microliters, at least 100 microliters, at least 300 microliters, at least 500 microliters, at least 1 mL, at least 3 mL, at least 5 mL, at least 10 mL, at least 30 mL, at least 50 mL, or at least 100 mL. In some cases, the droplet may have a volume of no more than 100 mL, no more than 50 mL, no more than 30 mL, no more than 10 mL, no more than 5 mL, no more than 3 mL, no more than 1 mL, no more than 500 microliters, no more than 300 microliters, no more than 100 microliters, no more than 50 microliters, no more than 30 microliters, no more than 10 microliters, etc. Combinations of any of these are also possible. For example, the droplet containing the cells may have a volume of between 1 mL and 3 mL.

In some cases, a portion of the droplet containing the cells may be removed, for example, to be assayed, to be removed as waste (e.g., to a waste region), or the like. In some cases, the droplet containing the cells may be divided or split, e.g., using manipulation techniques such as is described herein. For instance, one portion of the droplet may be removed while another portion remains at a cell culture region.

The cells may be cultured for any suitable length of time. For example, the cells may be cultured for at least 12 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, etc. In addition, in some cases, the cells may be cultured for no more than 14 days, no more than 13 days, no more than 12 days, no more than 11 days, no more than 10 days, no more than 9 days, no more than 8 days, no more than 7 days, no more than 6 days, no more than 5 days, no more than 4 days, no more than 3 days, no more than 2 days, no more than 1 day, etc. Combinations of these are also possible in some embodiments, e.g., cells may be cultured for between 4 and 8 days.

In certain embodiments, some or all of the cells can be adherent cells. Adherent cells may be cultured such that they adhere to the cell culture region. Thus, in some cases, one or more of the cell culture regions may be hydrophilic, e.g., to promote cell adhesion. For example, the surface of the DMF device may be naturally hydrophilic, and/or may be treated in some fashion to render in hydrophilic. The hydrophilicity may be a hydrophilicity that promotes cell adhesion, and those of ordinary skill in the art will be able to identify suitably hydrophilic surfaces that can be used to culture cells, such as mammalian cells.

Examples of surfaces that may be suitable for adhering cells include, but are not limited to, glass, polystyrene, or the like. In some cases, the surface, or a portion thereof (e.g., a cell culture region) may be hydrophilic, and/or treated to render it hydrophilic. For example, the surface (or portion thereof) may be treated with vitronectin, fibronectin, collagen, laminin, mucopolysaccharides such as heparin sulfate, hyaluronidate and chondroitin sulfate, synthetic polymers such as poly-D-lysine, or the like. In some cases, the surface may have 2, 3, or more cell culture regions, and these may independently be treated the same or differently (including untreated).

In addition, in certain cases, the substrate may be cleaned, e.g., with an RCA solution, to render the cell culture regions hydrophilic. An RCA solution may comprise, for example, deionized (DI) water, ammonium hydroxide, and hydrogen peroxide (e.g., as in RCA SC-1 solution). RCA solutions are known to those of ordinary skill in the art, and can be obtained commercially. Other types of cleaning solutions may also be used.

In one aspect, some or all of the cells within the DMF device may be genetically manipulated, e.g., using the DMF device to manipulate the cells to expose them to reagents that are able to genetically alter at least some of the cells. However, it should be understood that any of a variety of systems may be used within the DMF device to induce genetic manipulation; for example, electroporation, heat shock, viral delivery. Examples of these are discussed herein.

For example, in certain embodiments, genetic manipulation may be caused within the cells within the DMF device by exposing the cells to a suitable transfection complex, or other complex that can be used to cause genetic manipulation of the cells. For example, a transfection complex may be delivered to the cells within a cell culture region to cause transfection to occur. The transfection complex may be present, e.g., in a droplet that can be brought to the cell culture region, for instance, using droplet manipulation techniques such as those discussed herein. The transfection complex may be stored in a reservoir or a reagent region, which may be contained within the DMF device, for instance, on a surface within the DMF device used for culturing cells, or from a separate location, which may be a reservoir contained within the DMF device (e.g., within a bottle or other container), or separate from the DMF device, etc. (for example, pumped or otherwise delivered into the DMF device, e.g., as needed) According to certain embodiments, the transfection complex can include various lipids and/or polymers. For example, positively-charged liposomes or other vesicles can bind with the negatively charged DNA, and certain types of polymers can be designed that interact with DNA, e.g., to form lipid and/or polymer transfection complexes that can be taken up by the cells. In some cases a combination of lipids and polymers can also be used.

A variety of lipid transfection complexes are commercially available. These may include one or more lipids that can form liposomes or other vesicles, as noted above. In some cases, some of the lipids are positively charged. Non-limiting examples include Lipofectamine®, Lipofectamine® 2000, Lipofectamine® 3000, N-(1-(2,3-dioleyl-oxy)propyl)-N,N,N-trimethylammonium chloride (DOTMA), 2,3-dioleyloxy-N-(2-(sperminecarboxamido)ethyl)-N,N-dimethyl-1-propanaminium trifluoroacetate (DOSPA), TurboFect™, SuperFect®, jetPrime®, Lipofectamine® CRISPRMax™ Cas9, or the like. In addition, it should be understood that the invention is not limited to lipid transfection complexes; other types of transfection methods may be used, such as viral transfection or chemical transfection (for example, using calcium phosphate, e.g., to cause co-precipitation).

In some cases, the droplet containing the transfection complex may be diluted, e.g., in media, or in a liquid such as is described herein. In some embodiments, the complex may be diluted at a ratio of at least 1:1, at least 1:2, at least 1:3, at least 1:5, at least 1:7, at least 1:10, at least 1:20, etc., as determined by volume. In some cases, the dilution ratio may be no more than 1:10, no more than 1:7, no more than 1:5, or no more than 1:3. Combinations of these dilution ratios are also possible in some cases, e.g., a dilution ratio of between 1:1 and 1:10.

In one set of embodiments, such systems may be used to deliver one or more nucleic acids to the cells. For example such nucleic acids may be present as one or more plasmids, which can be uptaken by the cell, and optionally expressed within the cell. One example of such a system is the CRISPR-Cas9 genetic engineering system. Those of ordinary skill in the art would be well-aware of systems such as these. Briefly, in such a system, a plasmid delivered to a cell may encode a gRNA and/or an sgRNA sequence, and optionally a fluorescence reporter, such as mCherry, GFP, or RFP. The gRNA is a short synthetic RNA comprising a sequence for Cas binding, and a target sequence that defines a genomic target to be modified. In some cases, the plasmid may also encode Cas9 (which may be expressed intracellular to produce the Cas9 protein therein), and/or the Cas9 protein (or other proteins) may be separately delivered. Once expressed, the Cas9 protein and the gRNA form a ribonucleoprotein complex through interactions between the gRNA scaffold and surface-exposed positively-charged grooves on Cas9. Cas9 undergoes a conformational change upon gRNA or sgRNA binding that shifts the molecule from an inactive, non-DNA binding conformation into an active DNA-binding conformation. Importantly, the spacer region of the gRNA remains free to interact with target DNA. After binding of the gRNA or sgRNA to its target, e.g., within the genome, Cas9 will bind to and cleave the genome, which can be subsequently repaired, but can be used to cause gene inactivation or the introduction of heterologous genes through non-homologous end joining or homologous recombination, knock-out, knock-down, or knock-in of a gene, base editing, CRISPR activation, etc.

It should be understood that the plasmid may contain other sequences, e.g., in addition and/or instead of the ones described above. For example, in one embodiment, the plasmid may comprise cr:tracrRNA. In addition, other systems that can be used include other nucleic acid binding endonucleases or CRISPR-associated nucleases, such as, but not limited to, Cas12a/Cpf1, CRISPR-Cas13a, Cas13a/C2c2, Cas13b, Cas13c, and Cas13d, etc. Still other examples of nucleic acids include, but are not limited to, RNP (ribonucleoprotein) complexes, HDR (homology-directed repair) templates, base editing reagents, or the like.

In addition, it should be understood that other methods may be used to deliver one or more nucleic acids to the cells, in other embodiments of the invention. Examples include, but are not limited to, heat shock, calcium phosphate delivery, electroporation, nanoparticles, biolistics, microinjection, sonoporation, photoporation, magnetofection, viral delivery, or the like. For example, in some cases, relatively high voltages may be applied to cells, e.g., within a cell culture region, sufficient to cause delivery of nucleic acids into the cells. Those of ordinary skill in the art will be familiar with these and other techniques for delivering nucleic acids.

In one set of embodiments, a transfection complex may be created by combining a first transfection reagent with a second transfection reagent. For example, a first droplet containing the first transfection reagent may be fused with a second droplet containing the second transfection reagent, e.g., using droplet manipulation techniques such as those discussed herein, to form a combined droplet. In some cases, this may be performed in a mixing region within the DMF device. The first transfection reagent and the second transfection reagent may combine, e.g., spontaneously, to form the transfection complex. For example, the first transfection reagent may comprise a lipid while the second transfection reagent may comprise a nucleic acid. As a non-limiting example, the lipid may be one that is able to form a liposome, while the nucleic acid may be a plasmid, such as one that can modify the genome via CRISPR-Cas9. In some embodiments, however, the transfection complex may be introduced to the DMF device already prepared, e.g., no formation of the transfection complex within the DMF device may be performed.

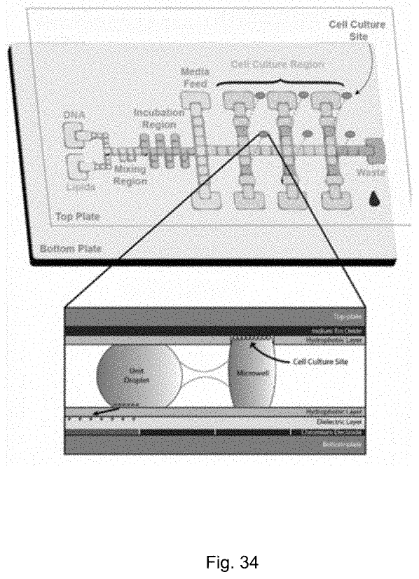

As a non-limiting example, in FIG. 34, a non-limiting example of a DMF device is shown. A first fluid containing DNA (e.g., a plasmid) may be introduced to a first reagent region, and optionally stored there before use, e.g., for any suitable length of time. Similarly, a second fluid containing a lipid may be introduced to a second reagent region, and optionally stored there before use, e.g., for any suitable length of time. A first droplet may be created from the first reagent region (e.g., containing DNA), and a second droplet may be created from the second reagent region (e.g., containing lipid), e.g., using droplet manipulation techniques such as those discussed herein. These droplets may then be moved to a mixing region, and fused together at the mixing region to form a combined droplet. In some cases, the first reagent and the second reagent may combine, for instance, spontaneously, to form a suitable transfection complex.

After formation, a droplet containing a suitable transfection complex may be dispensed to a cell, e.g., within a cell culture region within the device. As can be seen in FIG. 34, more than one cell culture region may be present within the device in certain instances. The droplet may have any suitable size or volume. For example, the droplet may have a volume of at least 10 microliters, at least 30 microliters, at least 50 microliters, at least 100 microliters, at least 300 microliters, at least 500 microliters, at least 1 mL, at least 3 mL, at least 5 mL, at least 10 mL, at least 30 mL, at least 50 mL, or at least 100 mL. In some cases, the droplet may have a volume of no more than 100 mL, no more than 50 mL, no more than 30 mL, no more than 10 mL, no more than 5 mL, no more than 3 mL, no more than 1 mL, no more than 500 microliters, no more than 300 microliters, no more than 100 microliters, no more than 50 microliters, no more than 30 microliters, no more than 10 microliters, etc. Combinations of any of these are also possible. For example, the droplet containing the cells may have a volume of between 1 mL and 3 mL.

The droplet may in some cases be introduced to the media containing the cells. For example, the media may be present as a droplet at the cell culture region, and the two droplets may be merged or fused together to expose the transfection complex to the cells, e.g., using droplet manipulation techniques such as those discussed herein. In some cases, the transfection complex is able to act on the cells spontaneously. However in other embodiments, delivery may be assisted, for example, using heat, electricity, sound, light, or the like. In some cases, for example, electricity or heat may be delivered to the cell culture region using suitable electrodes or other electronic components (e.g., resistive heaters, sensors, etc.) that may be contained within the surface of the DMF device.

In some embodiments, after transfection, the cells may be manipulated in some fashion. For example, the cells may be induced to produce a protein (e.g., one that was transfected into the cell), or to study various cell properties, for example by assaying cells using an assay region or area within the DMF device.

As a non-limiting example, after transfection a cell may be induced to cause expression of a nucleic acid. For example, IPTG (isopropyl-beta-D-1-thiogalactopyranoside) is an effective inducer of protein expression that triggers transcription of the lac operon, and can be used to induce E. coli protein expression where the gene is under the control of the lac operator. In some cases, the cells may be exposed to an inducer to induce the cells to express the inducible gene, for example, when the absorbance surpasses an absorbance threshold. In one set of embodiments, an induction region in the DMF device may be used to cause induction of a protein. For example, an induction region may comprise a hydrophilic site between a cell culture region and a mixing region or a reservoir for mixing cell culture and an induction reagent. The inducer may be present, for example, in a reservoir within the DMF device, and may be added to the cells and/or to a droplet (for example, to later be added to a droplet containing the cells) in the digital microfluidic device.

Another aspect is generally directed to digital microfluidics devices and methods for transfecting cells, including mammalian and bacterial cells. Those of ordinary skill in the art will be aware of DMF devices in general, and techniques for droplet manipulation, including dispensing, moving, splitting and mixing the droplets on the digital microfluidics device. It should be understood that DMF devices include a variety of techniques such as EWOD (electrowetting-on-dielectric) for manipulating droplets within the device. The substrate that the droplets are on may be, for example, glass, plastic, silicon, PCB (printed circuit boards), or the like. In addition, DMF devices may be obtained commercially in some instances.

In one set of embodiments, a DMF device may have a surface comprising a grid of locations in which droplets can be manipulated, e.g., using suitable electric fields applied to such locations. By suitable application of electric fields, a droplet on the surface of the DMF device may be moved from one location to another (e.g., from one location to an adjacent location), or a droplet may be created from a reservoir of fluid. For example, a droplet may be moved by sequentially actuating electrodes in a surface to cause or control movement of a droplet, e.g., to different locations. In addition, in one embodiment, a droplet may be split into two droplets, e.g., by applying substantially equal and opposite electrical forces to a droplet. In yet another embodiment, two droplets may be merged together, e.g., by applying electric fields to one or more droplets to cause them to collide in some fashion. Other droplet manipulation operations are also possible in other embodiments. These may include, but are not limited to, mixing the droplet (e.g., mixing the contents of the droplet and/or mixing the droplet with another droplet), diluting the droplet, incubating the droplet, culturing cells within the droplet, performing knock out experiments on cells within the droplet, and performing transfection experiments on cells within the droplet.

As an example, in one set of embodiments, a droplet may be mixed within a mixing region in the DMF device, e.g., such that the contents of the droplet are mixed, and/or to mix the droplet with another droplet. In some cases, for example, mixing may be performed by moving a droplet back and forth between two locations (e.g., repeatedly), moving the droplet in a square or rectangular pattern, or the like, which may cause active mixing of the droplet to occur.

A location for containing a droplet on the DMF device, e.g., as arranged into a grid, may independently have any suitable shape or size. For example, a location may be square, circular, rectangular, triangular, hexagonal, or the like. A given location may have regular or irregular shapes. In addition, in some instances, the boundary between different adjacent locations may be interlaced together. In some cases, a location may have a diameter or other characteristic dimension of at least 0.1 mm, at least 0.2 mm, at least 0.3 mm, at least 0.5 mm, at least 1 mm, at least 1.5 mm, at least 2 mm, at least 3 mm, at least 5 mm, at least 10 mm, at least 20 mm, at least 30 mm, at least 50 mm, at least 100 mm, etc. In certain instances, the location may have a diameter or other characteristic dimension of no more than 10 mm, no more than 5 mm, no more than 3 mm, no more than 2 mm, no more than 1 mm, no more than 0.5 mm, no more than 0.3 mm, no more than 0.2 mm, no more than 0.1 mm, etc. In addition, it should be understood that combinations of any of these are also possible in various embodiments. For example, a location may have a diameter or other characteristic dimension of between 10 mm and 50 mm.

In one set of embodiments, droplet manipulation operations may be programmed within the DMF device using a suitable interface, e.g., as discussed herein. The interface may be able to operate a control unit that can apply suitable electrical forces to a surface upon which the droplet is present, which may be used to perform the various droplet manipulation operations. The interface may also be able to track droplet movements and/or visualize droplet manipulations in certain cases. For example, the interface may present the surface as a grid of locations, and a user may manipulate droplets on the grid via the interface, which can send suitable signals to the control unit to thereby manipulate the droplets. Thus, for example, a user can provide, through the interface, a set of instructions to the control unit for dispensing, moving, splitting and mixing the droplets on the digital microfluidics device. In some cases, the user can build a grid corresponding to a device grid of the digital microfluidics device, and/or generates a sequence of droplet operations on the grid. For example, in certain embodiments, the user may be able to import a sequence of droplet operations to the digital microfluidics device, and the interface may be able to provide a set of instructions to the controls unit for executing such a sequence of droplet operations on the device grid of the digital microfluidics device.

In certain embodiments, the DMF device comprises a surface or plate upon which 1, 2, 3, 4, 5, or more droplets may be present. One or more of the droplets may be manipulated, e.g., independently, as discussed above. The droplets may be manipulated sequentially and/or simultaneously, depending on the application.

A surface or plate may comprise, in one set of embodiments, materials such as glass, polymers such as polystyrene, polyethylene terephthalate (PET), etc., FR4 or other composite materials comprising a woven fiberglass cloth and an epoxy resin binder, paper, or semiconductors such as silicon, GaAs, or the like. In some cases, the surface may be coated or patterned with a metal, e.g., forming a metal-coated substrate or a metal-patterned surface. For example, the metal may include aluminum, silver, gold, platinum, chromium, indium, tin, copper, or the like. Alloys of these and/or other metals are also possible. As another example, in one embodiment, the surface may comprise an electrode comprising indium tin oxide (ITO). ITO may be particularly useful, for example for creating surfaces that are at least partially transparent. For example, in one embodiment, a surface may comprise an electrode formed from an indium tin oxide (ITO) coated glass substrate.

In addition, in some cases, there may be a second surface, e.g., substantially parallel to the first surface. For example, the surfaces may be positioned such that the first surface is a top plate and the second surface is a bottom plate, e.g., defining a space between the plates for droplets. In some cases, fluids or droplets may be conveyed or transferred from one surface to another, e.g., using gravity and/or electrical forces. In other embodiments, the spacing between the surfaces may be sufficiently small that a droplet is in contact with both surfaces simultaneously. The first and second surfaces may comprise the same or different materials. For example, in one embodiment, one surface may be hydrophilic and one surface may comprise a dielectric, e.g., as discussed herein.

Accordingly, there may be any suitable spacing between the first and second surfaces, depending on the application. For example, the spacing may be at least 5 micrometers, at least 10 micrometers, at least 20 micrometers, at least 30 micrometers, at least 50 micrometers, at least 100 micrometers, at least 130 micrometers, at least 200 micrometers, at least 300 micrometers, at least 500 micrometers, at least 1 mm, at least 2 mm, at least 3 mm, at least 5 mm, at least 10 mm, at least 20 mm, at least 30 mm, at least 50 mm, at least 100 mm, etc. In some cases, the spacing may be no more than 100 mm, no more than 50 mm, no more than 30 mm, no more than 10 mm, no more than 5 mm, no more than 3 mm, no more than 1 mm, no more than 500 micrometers, no more than 300 micrometers, no more than 204 micrometers, no more than 180 micrometers, no more than 150 micrometers, no more than 100 micrometers, no more than 50 micrometers, no more than 30 micrometers, no more than 10 micrometers, etc. Combinations of any of these are also possible. For instance, the spacing may be between 5 micrometers and 240 micrometers, between 100 micrometers and 180 micrometers, between 130 micrometers and 150 micrometers, etc. In certain embodiments, the spacing may be controlled by a spacer. For example, in one embodiment, the separation material comprises a dielectric spacer to form an inner channel for supporting and transporting droplets and/or delivering fluids to reservoirs. In some cases, one or more of the plates may be detachable.

In some cases, a surface may be treated to promote, or discourage, cell adhesion, e.g., as discussed herein. For example, the surface, or a portion thereof (e.g., a cell culture region) may be hydrophilic, and/or treated to render it hydrophilic, e.g., with vitronectin, fibronectin, collagen, laminin, mucopolysaccharides such as heparin sulfate, hyaluronidate and chondroitin sulfate, synthetic polymers such as poly-D-lysine, etc. For example, a hydrophilic surface may exhibit a contact angle of less than 900. In addition, in some cases, a surface, or portion thereof may be hydrophilic, and/or be treated to render it hydrophilic.

In some cases, a surface may be used to integrate other electrodes for transformation or transfection experiments on the microfluidic device. In addition, a surface may be used to exchange of reagents on the microfluidic device. In some cases, a surface can hold magnetic beads while exchanging liquid on the microfluidic device.

In addition, in one set of embodiments, the surface may contain one or more electrodes, sensors, or the like, e.g., as discussed herein. For example, the electrodes may, when actuated, control movement of a droplet on the surface, or otherwise manipulate droplets. For example, the electrodes may be used to move a fluid, e.g., a droplet to an assay region or assay area, for example, for measurement by an optical density reader. The electrodes may, in some cases, comprise dielectric and/or hydrophobic layers. In some cases, the electrodes may be metal-patterned or metal-coated. In certain embodiments, for example, the electrodes may be formed on an electrically insulating substrate, where the electrode is coated or patterned with a dielectric layer having a hydrophobic surface. In addition in certain embodiments, an electrode layer may be supported by an electrically insulating substrate.

In addition, in some cases, the surface may include a dielectric layer or coating and/or a hydrophobic layer or coating. Such layers or coatings may cover all, or only a part of, the surface; for example, the layer or coating may not cover a cell culture region or the surface. For instance, in one embodiment, the dielectric layer comprises Parylene C, or a poly(monochloro-para-xylylene). In addition, a hydrophobic coating or layer may be present, and in some cases, may cover some or all of the dielectric. Non-limiting examples of hydrophobic coatings include FluoroPel® PFC1601V or a fluoroacrylic copolymer. The coatings may be applied to the surface using any suitable technique, including spin-coating, dip-coating, deposition techniques such as CVD (chemical vapor deposition), or the like.

Furthermore, in some embodiments, the hydrophobic coating may comprise a surfactant. Non-limiting examples include poloxamers or nonionic triblock copolymers composed of a central hydrophobic chain of polyoxypropylene (poly(propylene oxide)) flanked by two hydrophilic chains of polyoxyethylene (poly(ethylene oxide)). Specific non-limiting examples include Pluronic® F68, Pluronic® P105, Pluronic® F127, or the like. Without wishing to be bound by any theory, it is believed that in some cases, a surfactant may help to reduce surface tension, e.g., of the droplets on the surface. A surfactant may be present at any suitable concentration, for example at at least 0.01 vol %, 0.02 vol %, 0.03 vol %, 0.05 vol %, 0.1 vol %, 0.2 vol %, 0.3 vol %, 0.5 vol %, 1 vol %, 2 vol %, etc., and/or at no more than 1 vol %, no more than 0.5%, no more than 0.3%, no more than 0.2%, no more than 0.1%, no more than 0.05%, no more than 0.03%, no more than 0.02%, no more than 0.01%, etc.

In some cases, one or more surfaces may be cleaned, for example, by exposure or immersion in an RCA solution. For example, the RCA solution may comprise DI water, aqueous ammonium hydroxide, and/or hydrogen peroxide. Other types of cleaning solutions may also be used. In some cases, clearing may be used to render the cell culture regions hydrophilic.

In addition, in certain cases, photoresist may be present on at least a portion of the surface (e.g., on at least a portion of the dielectric and/or hydrophobic layer or coating. This may be useful, for example, for creating cell culture regions such as those discussed herein.

In one set of embodiments, the DMF device also contains one or more cell culture regions or areas, e.g., for culturing cells, such as mammalian cells or bacteria. In some cases, a cell culture region may include a hydrophilic site is configured for dispensing a composition for culture. In some cases, the cell culture region may include a hydrophilic site fabricated with an electrode and used for cell sensing.

In some cases, there may be 2, 3, 4, 5, 6, 7, 8, 9, 10, or more cell culture regions, and such cell culture regions may be present on any surface of the DMF device (e.g., on a bottom or a top plate). In some cases, cells may be introduced or seeded onto a DMF device, e.g., and delivered or dispersed to one or more of the cell culture regions, e.g., using droplets such as discussed herein. The cells in some cases may then be allowed to adhere to the culture regions.

The cell culture regions may independently have any suitable shape or size, e.g., for culturing one or more cells. For example, a cell culture region may be square, circular, rectangular, triangular, hexagonal, or the like. The cell culture regions may have regular or irregular shapes. In some cases, a cell culture region may have a diameter or other characteristic dimension of at least 0.1 mm, at least 0.2 mm, at least 0.3 mm, at least 0.5 mm, at least 1 mm, at least 1.5 mm, at least 2 mm, at least 3 mm, at least 5 mm, at least 10 mm, at least 20 mm, at least 30 mm, at least 50 mm, at least 100 mm, etc. In certain instances, the cell culture region may have a diameter or other characteristic dimension of no more than 10 mm, no more than 5 mm, no more than 3 mm, no more than 2 mm, no more than 1 mm, no more than 0.5 mm, no more than 0.3 mm, no more than 0.2 mm, no more than 0.1 mm, etc. In addition, it should be understood that combinations of any of these are also possible in various embodiments; for example, the diameter or other characteristic dimension may be between 1 mm and 2 mm, between 0.1 mm and 5 mm, or the like.

The cell culture regions may be treated to allow cells to adhere to them. If more than one cell culture region is present, the cell culture regions may have the same or different treatments. In addition, any suitable technique may be used to create a cell culture region. As a non-limiting example, a surface may be exposed through the photomask with an array of six 1.75 mm diameter circular features, and optionally, after rinsing, air-drying and dehydrating, the top-plate is then flood exposed, spin-coated with Teflon, and post-baked. In addition, in some embodiments, after being allowed to cool, the surface may be immersed in acetone with agitation until the Teflon-AF over patterned sites is lifted off, and optionally, after being rinsed with DI water and dried under a stream of nitrogen, droplets of AZ300T stripper are placed over the spots and the substrates are placed aside followed by rinsing with DI water and air-drying; and optionally post-baking followed to reflow the Teflon-AF.

In addition, in one set of embodiments, at least some of the cells may be removed or detached from the cell culture region, e.g., for subsequent use and/or analysis, for example, by an optical density reader, or other assay technique, such as those described herein. In some cases, for example, for adherent cells, a removal agent may be applied to the cells to remove at least some of the cells. Non-limiting examples include trypsin and other cell-dissociation enzymes, and/or EDTA. In some cases, such removal agents may be contained in droplets and introduced to the cells, e.g., using droplet manipulation techniques such as those discussed herein.

Various reagents may be added to the DMF device, depending on the application. For example, cell media, transfection reagents, removal agents, waste, or the like may be added and/or removed from the DMF device. In some cases, a reagent may be stored within or separate from the device, e.g., in a reservoir, and introduced as needed. In some cases, a reservoir or other region may be present, e.g., on the surface of the DMF device, for storage purposes, then delivered from the reservoir to other locations, e.g., a cell culture region, as discussed herein. Examples of such regions include, but are not limited to, media feed regions, reagent storage regions, or the like.

Regions such as media feed regions, reagent storage regions, cell culture regions, incubation regions, mixing regions, waste regions, assay regions, assay reagent regions etc. may each independently have any suitable shape and/or size, in accordance with another set of embodiments. In addition, any number of any of these may be present, e.g., there may be 2, 3, 4, 5, 6, 7, 8, 9, 10, or more of a type of region present on the surface of the DMF device.

The regions on the DMF device may independently have any suitable shape or size. For example, a region may be square, circular, rectangular, triangular, hexagonal, or the like. The regions may each have regular or irregular shapes. In some cases, a region may have a diameter or other characteristic dimension of at least 0.1 mm, at least 0.2 mm, at least 0.3 mm, at least 0.5 mm, at least 1 mm, at least 1.5 mm, at least 2 mm, at least 3 mm, at least 5 mm, at least 10 mm, at least 20 mm, at least 30 mm, at least 50 mm, at least 100 mm, at least 200 mm, at least 300 mm, at least 500 mm, etc. In certain instances, the region may have a diameter or other characteristic dimension of no more than 500 mm, no more than 300 mm, no more than 200 mm, no more than 100 mm, no more than 50 mm, no more than 30 mm, no more than 20 mm, no more than 10 mm, no more than 5 mm, no more than 3 mm, no more than 2 mm, no more than 1 mm, no more than 0.5 mm, no more than 0.3 mm, no more than 0.2 mm, no more than 0.1 mm, etc. In addition, it should be understood that combinations of any of these are also possible in various embodiments. For example, a region may be between 1 mm and 2 mm, between 10 mm and 50 mm, or the like.

In accordance with one set of embodiments, one or more assays may be performed, e.g., on droplets within the DMF device. In some cases, one or more droplets may contain cells, e.g., from the cell culture region. In certain embodiments, for example, cells adherent to the cell culture region may be removed or detached using a removal agent, such as is discussed herein.

One or more droplets may be moved to an assay region or assay area, e.g., using various droplet manipulation techniques including those discussed herein. The assay region may include any of a variety of systems that can be used to determine a droplet, e.g., quantitatively or qualitatively. For instance, properties such as the size, fluorescence, opacity, transparency, weight, viscosity, light intensity, resistivity, capacitance, absorbance, or the like may be determined, e.g., using one or more sensors. The sensors may be contained within one or more surfaces of the DMF device, and/or separate from the surface. In some cases, a microscope, such as a light microscope, may be used to determine a portion of the DMF device, such as an assay region or assay area.

In some cases, the assay region or assay area may include a hydrophilic site or region, e.g., for presenting the at least a portion of the cell culture to the optical density reader. In certain embodiments, the hydrophilic site or region may be prepared as a cell culture region, e.g., as discussed herein. Thus, as a non-limiting example, in one embodiment, a microfluidic device may comprise a first plate or surface comprising a cell culture region (e.g., for maintaining a cell culture), an optical density or assay region (e.g., for measuring an optical density of at least a portion of the cell culture), and a hydrophilic site positioned between the cell culture region and the optical density reader. The hydrophilic site may be used for presenting at least a portion of the cell culture to the optical density reader. The device may also comprise a second plate or surface comprising electrodes that, when actuated, control movement of the cell culture to the hydrophilic site, e.g., to be measured at the assay region or by the optical density reader.

In one embodiment, an optical signal of a droplet may be determined at the assay region or assay area. Non-limiting examples include fluorescence, opacity, transparency, light intensity, optical density (OD), absorbance, or the like. The optical signal of the droplet may be used to determine one or more parameters within a droplet, for example, cell activity, transfection efficiency, enzyme activity, or the like.

For instance, as a non-limiting example, cells may be determined after genetic manipulation (for example, using CRISPR-Cas9 or other techniques to transfect or induce cells) to determine the success of the genetic manipulation. For instance, the genetic manipulation may include a fluorescence reporter that is introduced into the cells, and cells able to express the fluorescence reporter may be determined to determine the success of the genetic manipulation.

An optical signal can be determined, for example, using a light emitting source and sensor or a microscope, and may be positioned to determine any portion or region of a surface of the DMF device, for example, a cell culture region or an assay region. For instance, in one embodiment, the optical density (OD) of a droplet may be determined, for example, using an optical density (OD) reader, which may allow monitoring of the optical density of the droplet.

The light emitting source may be able to emit in any suitable frequency, e.g., light, infrared, ultraviolet, etc. The light emitting source may emit at one or more frequencies. For instance, the light emitting source may emit light at 400 nm, 500 nm 600 nm, 700 nm, etc. Higher or lower frequencies are also possible. Any suitable light emitting source may be used, e.g., an LED, many of which are commercially available. Similarly, any sensor may be used, such as a photomultiplier tube, a photoelectric sensor, a photodiode, a CMOS chip, a CCD device, or other photodetectors. In some cases, one or more of these can be placed in optical communication with a surface, e.g., directly, or indirectly (for example, using optical fibers).

In some cases, more than one surface may be present within a DMF device, as discussed herein, and in some embodiments, one (or more) of the surfaces may be at least partially transparent (or at least a portion of the surface), to allow determination of droplets or regions within the DMF device to occur. For instance, a surface may comprise glass, ITO, etc., which may be useful to allow an optical signal of a droplet to be determined from the DMF device.

As a non-limiting example, in certain embodiments, the assay region may comprise an absorbance reading electrode, e.g., within a surface, the absorbance reading electrode comprising a transparent section such that the optical density reader can determine a sample positioned at or proximate the absorbance reading electrode. The transparent section may be in the middle, center, or edge of the absorbance reading electrode. A light emitting source can be placed above the absorbance reading electrode and the sensor can be placed on or above the absorbance reading electrode for monitoring of the optical density of the assay region. In some cases, the light emitting source can be placed above the transparent window of the absorbance reading electrode and the sensor can be placed below the transparent window for reading intensity of the light passing emitted by the light emitting source. The absorbance reading electrode may comprise any suitable width and length, for example, a width of about 2.25 mm and a length of about 2.25 mm. The transparent section can also comprise any suitable width and length, for example, a width of about 0.75 mm and a length of about 0.75 mm.

In addition, in one set of embodiments, the DMF device may include a computer vision system for acquiring images of a surface of the DMF device, or at least a portion thereof. The computer version system may be part of a control unit or interface, e.g., within the DMF device. A variety of computer vision systems are available commercially. The computer vision system can be used to determine a variety of conditions of the DMF device or portions thereof, such as the media feed region, the cell culture region, the incubation region, the waste region, the reagent storage region, the assay region, or other regions or areas such as those discussed herein. For example, in some cases, the computer vision system can be used to detect the size of a droplet, which may be useful for determining droplet manipulation failures, e.g., failures in dispensing, movement, splitting, merging, etc. of droplets on the digital microfluidics device.

Examples of imaging techniques that can be used include, but are not limited to, feature extraction techniques such as independent component analysis, isomaps, kernel PCA, latent semantic analysis, partial least squares, principal component analysis, multifactor dimensionality reduction, nonlinear dimensionality reduction, multilinear principal component analysis, multilinear subspace learning, semidefinite embedding, autoencoders, or the like. Additionally, non-limiting examples of image processing techniques that can be used include edge detection, corner detection, blob detection, ridge detection, scale-invariant feature transform, edge direction, changing intensity, autocorrelation, motion detection, thresholding, blob extraction, template matching, Hough transforms, or the like.

As a non-limiting example, an image-based feedback system may be operated by resting a droplet on a first electrode, applying a potential to a second electrode, capturing a frame after actuation, creating a difference frame by taking the difference from a grayscale image and a reference image (e.g., no dispensed droplets), creating a binarized frame from the difference frame, detecting circles from this frame through a Hough transform, and returning a successful or unsuccessful result depending on the location of the actuated droplet and a user-defined detection box.

Thus, in some cases, a portion of the DMF device, such as an assay region or a cell culture region, may be monitored, e.g., by a user, and/or automatically. For example, the monitoring may occur by taking images of the composition and analyzing the images, e.g., in the control unit, or on a computing device. As non-limiting examples, analyzing the images may include image cropping, identifying individual and overlapping cells, counting numbers of cells, measuring the size and shape of the cells, creating binary images of the cells, comparing knocked-out and non-knocked out cells, or the like.

In one set of embodiments, the DMF device includes a control unit. The control unit may allow for the determination and/or manipulation of droplets within the DMF device. In addition, in some cases, the control unit may be used to automate some or all of the steps for genetic manipulation, e.g., culture, delivery, analysis, etc., such as is discussed herein. For example, the control unit may allow a user to transfect cells, e.g., using CRISPR-Cas9, or other techniques for genetic manipulation, such as those described herein, and to determine the response of the cells, e.g., the transfection efficiency.

In some cases, such determinations of a droplet (e.g., using a computer vision system) may allow a control unit to take corrective action. For example, a control unit may be able to sense or determine droplets on an electrode or surface of the digital microfluidics device, e.g., using the computer version system, and control unit may control droplets on an electrode by applying a potential to the electrode. Determination that a failure of droplet manipulation has occurred may allow the control unit to take suitable corrective action, e.g., by re-applying the potential at an electrode or surface if the droplet is not present. Thus, in some cases, the computer vision system may determine droplet operations or manipulations on a surface of the DMF device, e.g., on a device grid, and may provide feedback to the interface or to the control system, e.g., to allow correction action to occur. The feedback may include image data, video data, and/or other types of data.

In some embodiments, the control unit may include an interface, such as a graphical user interface, for allowing a user to control the DMF device. Those of ordinary skill in the art will be familiar with a variety of different interfaces, including graphical user interfaces, that can be used to execute various droplet manipulation operations within the DMF device. For example, the interface may include a set of instructions written that allows a user to program the DMF device to execute various droplet manipulation operations. For example, a program may include a set of instructions to dispense and to move droplets, and/or to mix droplets with other droplets, e.g., for analysis. In some cases, a set of instructions may equate to a droplet operation (e.g., mix, dispense, split, etc.). Thus, in some embodiments, the interface may allow a user to program various droplet operations or manipulates, track droplet movements within the DMF device, visualize or monitor droplet manipulations on the digital microfluidics device, or the like.

As a non-limiting example, in some embodiments, a droplet may be introduced to an assay region, e.g., for determination by an optical density (OD) reader. Reagents may be loaded into the droplet, and a series of desired droplet movement steps such that induction (and/or cell culture and analysis) may be performed, e.g., using an interface and/or a control unit.

In addition, in one embodiment, as a non-limiting example of the use of an assay region, cells within the DMF device may be induced or transfected, optionally quenched, and the optical density of the cells may be read or otherwise determined, e.g., before and/or after inducement or transfection. In some cases, the optical density may be determined in order to induce the cells at an optimal value or a desired time. This may be useful, for example to determine when the cells are ready for genetic manipulation. In some cases, this may be programmed by a user using the control unit.

For example, an image-based feedback system may be used to induce protein expression by cells in a cell culture on a DMF device. In some cases, the DMF device may comprise a plate assembly having a first plate and a second plate, and the system may be able to monitor optical density of at least a portion of the cell culture, such that when the optical density reaches a threshold optical density, at least a portion of the cell culture may be moved to an assay region or a hydrophilic site. There, an inducing agent may be introduced to induce protein expression.

As another non-limiting example, a control system may detect or determine whether a droplet is located at a suitable region of a surface or electrode, e.g., a destination electrode, by instructing the computer vision to capture a frame of the position of the droplet on the surface or electrode, determining a difference image by subtracting a reference image from the frame to identify a boundary of the droplet, and detecting whether the droplet is on the electrode on the difference image. In some cases, if the droplet is not detected, the control unit can initiate feedback by actuating a source electrode of the droplet, actuating the destination electrode of the droplet, pausing for a predetermined amount of time, turning off the source electrode, incrementing the voltage at the electrode by a predetermined voltage amount, and turning off the destination electrode. Thus, the control unit may be able to determine or detect whether the droplet is located at its destination. In some cases, this may be programmed by a user using the control unit.

Accordingly, certain embodiments of the invention are generally directed to an image-based system for tracking droplet movement on a digital microfluidics device. In some cases, as a non-limiting example, the image-based system may comprise a computer vision system for capturing images of a droplet on an electrode or surface of the digital microfluidics device, a control unit configured to manipulate the droplet, and an interface coupled to the computer vision system and the control unit. The interface may be separate from, or part of, the control unit. In some cases, the interface unit may be configured and arranged to direct the control unit to manipulate the droplet, receive images of the droplet (for example, as captured by the computer vision system), and/or determine, based on the images captured by the computer visions system, a position of the droplet within the digital microfluidics device.

The following documents are incorporated herein by reference: Int. Pat. Apl. Ser. No. PCT/CA2018/051063, filed Sep. 4, 2018; U.S. Pat. Apl. No. 62/627,022 filed on Feb. 6, 2018; and U.S. Pat. Apl. No. 62/693,998 filed on Jul. 4, 2018.

The following examples are intended to illustrate certain embodiments of the present invention, but do not exemplify the full scope of the invention.

### Example 1

This example illustrates an image-based feedback and analysis system for digital microfluidics. This example illustrates a feedback system and method for digital microfluidics (DMF) devices that relies on imaging techniques that will allow detection of droplets. In some cases, this may be performed without the need to reactivate all destination electrodes. The system includes integrating electronics with a CMOS camera system and a zoom lens for acquisition of the images that may be used to detect droplets on the device. The system may also include an algorithm, e.g., running on suitable electronics within the device. The algorithm may use a Hough transform to detect a variety of droplet sizes, to detect singular droplet dispensing, and/or to detect movement failures of droplets within the device.

Digital microfluidics (DMF) is a technology that provides a means of manipulating nanolite-microliter volumes of liquids on an array of electrodes, e.g., as discrete droplets. By applying an electric potential to an electrode, these discrete droplets can be controlled, for example in parallel. For example droplets within the DMF device can be transported, mixed, reacted, and/or analyzed for a variety of applications. Typically, an automation system can be interfaced with a DMF device that uses a set of basic instructions written by the user to execute droplet operations. In some embodiments, there are provided feedback systems and methods for DMF device that rely on imaging techniques. The imaging techniques can allow detection of droplets, e.g., online and/or without the need to reactivate all destination electrodes.

The feedback system uses integrating electronics with a CMOS camera and a zoom lens for acquisition of the images that may be used to detect droplets on the device. The system can include a computer program or an algorithm. The algorithm may use a Hough transform to detect a variety of droplet sizes, to detect singular droplet dispensing, and/or to detect movement failures of droplets within the device. In some cases, for example, a system may be used to determine droplet movement for a variety of liquids used in cell-based assays and/or to optimize different feedback actuation schemes to improve droplet movement fidelity. The system may also be applied in certain embodiments to a colorimetric enzymatic assay for biological analysis. In some cases, an integrated imaging and feedback systems for DMF device can be used as a platform for automating biological assays and/or analyses.

Digital microfluidics (DMF) sallow the manipulation of droplets on an electrode array surface by the application of electric potentials. The DMF device may provide methods of manipulating droplets for a wide range of volumes, and different droplets may be independently transported, mixed, reacted, and analyzed. DMF devices may be used for integrating fluid handling for a vast range of applications requiring multiplexing, such as synthetic biology or clinical diagnostics applications. One advantage with digital microfluidics is that may be amenable to being integrated with various automation systems and/or to external detectors or internal in-line detectors, e.g., for biological analysis, which may be offline in some cases.

Typically, an automation system can be interfaced to a DMF device that accepts a standard set of basic instructions, e.g., written by the user, to execute various droplet operations. For example, a program may include a set of instructions to dispense and to move droplets, and/or to mix droplets with other droplets, e.g., for analysis. In some cases, a set of instructions may equate to a droplet operation (e.g., mix, dispense, split, etc.). However, in some cases, due to factors such as surface heterogeneity or the contents of the droplet, every application of a potential does not easily translate to a movement of a droplet on the device. This behavior may be exacerbated in certain cases when the droplet contains cells or proteins as they tend to “biofoul” the surface and render the device increasingly difficult or useless, even over a few actuations in some instances.

One solution that can alleviate these problems is to use a control feedback system since they provide a means to “sense” the droplet on the electrode. By sensing a droplet on the electrode, a control algorithm can be executed to re-apply the potential at a destination electrode if the droplet is not present on that electrode. This can be repeated until the droplet completes the desired operation. One commonly used scheme for sensing droplets on DMF devices is to use capacitive sensing, since the configuration of a DMF device can be electromechanically modeled with resistors and capacitors. There have been a few papers that describe the integration of a capacitive feedback system with digital microfluidics, including with ring oscillator circuits that use frequency changes in the applied signal to monitor droplet dispensing, a simple resistor and capacitor circuit to output voltage values which will be used to monitor droplet movement, or fuzzy control algorithms that compute optimized electrode charging time and real-time monitoring of the droplet on device. These methods use electronic circuits to sense and to monitor the droplet on device. However, a drawback with these methods is that these systems are not capable of detecting individual droplet failures. If a failure is detected, these systems require a re-application of a potential on the destination electrode for all the droplets on the device, since it is not known which droplet on the device has failed in operation. This is not a favorable solution, since excess activation of electrodes reduces the integrity of the dielectric and causes the surface to be prone to biofouling. Furthermore, these systems are only capable of sensing the droplet, but require external detectors (e.g., well-plate readers) for bioanalysis.

As an alternative to these different techniques, this example illustrates a digital microfluidic systems based on image-based techniques that can be used, for example, for feedback and analysis. While others have suggested the use of droplet tracking software, which tracks the droplet position, such devices do not provide feedback and analysis of the droplets on DMF devices. This system can include a camera with a focus zoom lens to monitor individual droplet movements. This system may be used, for example, to show multiplexed droplet dispensing and individual monitoring of droplet detection failure, to actuate a range of fluids that are useful for biological assays, and/or to validate that the system can be used for analyzing an enzymatic assay using colorimetric pixel detection. This system can be useful, for example, for users adopting DMF devices for various biological applications.

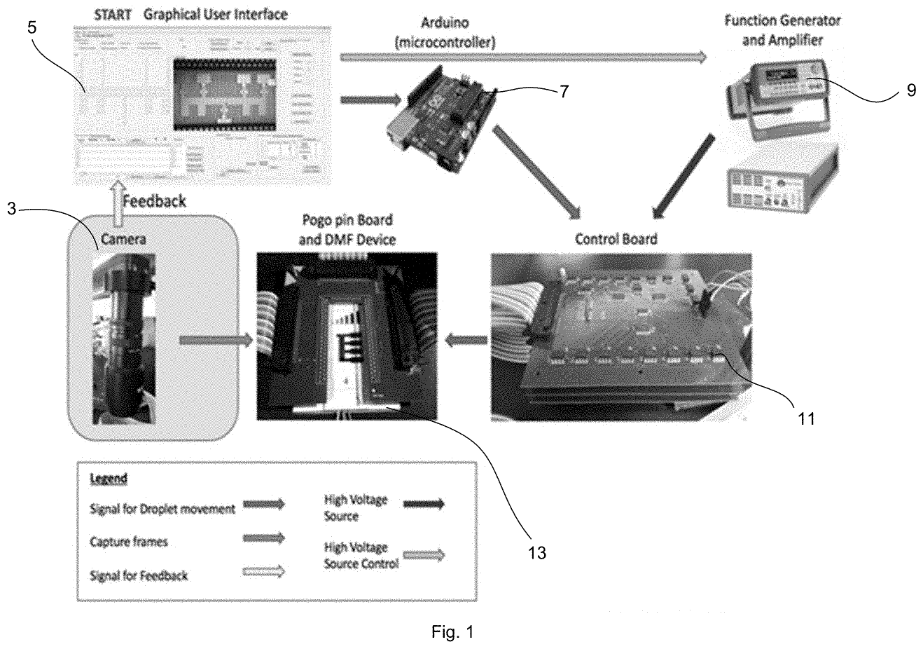

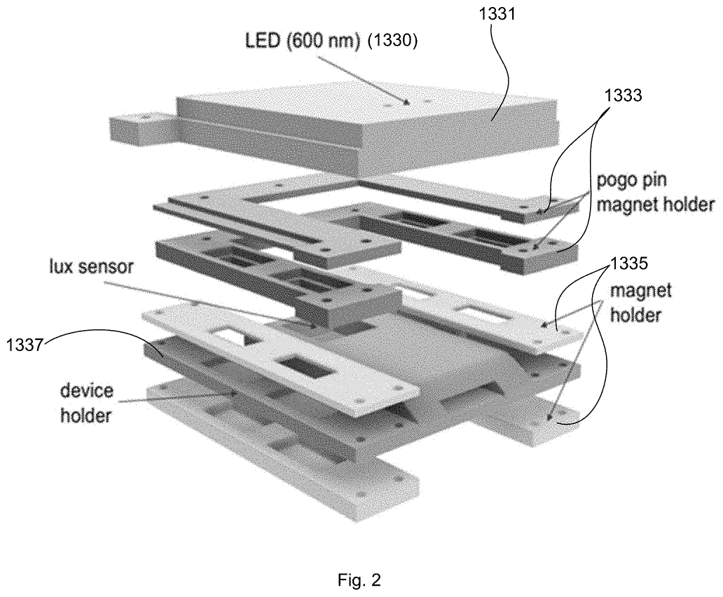

This feedback system and its setup is illustrated in FIG. 1. The digital microfluidic device in this example is attached to a pogo pin-control board with a 3D printed base platform (see FIG. 2) that delivers electric potentials to the device for droplet movement.

FIG. 1 illustrates a schematic of an image-based DMF feedback system. For example, the feedback system in this example has a computer vision system (e.g. a camera) 3, a graphical user interface (GUI) 5, a microcontroller (e.g. Arduino) 7, a function generator and amplifier 9, a switching control board 11, and a pogo pin board and DMF device 13. For example, the pogo pin board can be 3D printed based to control the application of electric potentials that is applied to the DMF device. The graphical user interface 5 can be programmed by the user to deliver a series of droplet actuations and acquires images to manage the control logic for the sequential application of electric potentials to the DMF device.

FIG. 2 describes the fabrication of an automated induction microfluidic system (AIMS) according to this example. The system has four layers (top to bottom): Layer 1 (1331) to hold the LED (1330); Layer 2 (1333) is to support the pogo pin board that will apply electric potentials to the device; Layer 3 (1335) is used to support the device in place; and Layer 4 (1337) is to position the sensor directly below the device.

For example, the pogo pin board may be a 2.5 mm thick board (printed by Gold Phoenix, Mississauga, ON) with surface mount pogo pins that will connect to the digital microfluidic device. These pogo pin boards are connected (e.g., via ribbon cable) to three control boards (printed by Gold Phoenix, Mississauga, ON) that houses 80 solid state switches on each board.

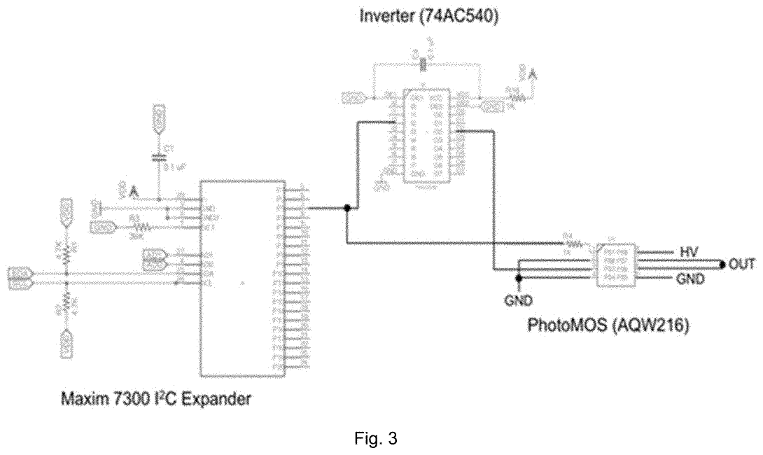

A typical output that connects to a pogo pin is configured to designate two states in this example: ground and high-voltage. Each switch may be controlled by an I/O expander that is used to deliver 5V power (e.g. logic high) to a switch via I2C connection from the Arduino and an inverter that will automatically deliver a logic low (e.g. ground voltage) to a switch for the same output to prevent any short circuit between power and ground (see FIG. 3).

This connection scheme is repeated to allow 104 outputs on the digital microfluidic device. The Arduino Uno microcontroller and high-voltage amplifier (Trek Ltd., PZD700A) are connected to the control board and the function generator (Allied Electronics, 33210A Agilent) and is connected to the computer via USB connection. The main component of the feedback imaging system is a 3.0 MP CMOS Color USB camera (Edmund Optics, EO-3112C) attached to a 10× C-mount Close Focus Zoom lens (Edmund Optics, 54363).

An additional lighting setup was configured around the camera and the device. This setup used a Fiber Optic Illuminator 150 W (Edmund Optics, 38939) with a 23 inch semi-rigid dual branch (Edmund Optics, 54212) that was directed onto a homemade backdrop. To acquire images, intensity of the fiber light was adjusted and the camera was rotated −5° from the vertical center to enhance the outline of the droplet. High-resolution images (2048×1536 pixels) were acquired and used for droplet analysis and detection.

Referring to FIG. 3, there is shown a circuit diagram showing the connectivity of one output that connects to a pogo pin. The software uses I2C communication protocol to deliver a user-configurable high (5V) and low (0V) signals to the Arduino (not shown). The data (SDA) and clock (SCL) signals are delivered to a Maxim I/O expander with an address AD0 and AD1 and the output of the expander is connected to a PhotoMOS switch and inverter. Each switch contains two optical photodiodes that will be used to deliver two logic states: high (e.g. ˜100 V) and low (e.g. 0 V). The inverter is used to prevent any short circuit at the output of switch. The output of the switch is connected to a pogo pin board that houses 104 spring loaded pins.

The feedback software setup of this example is now described. The Arduino system/controller is controlled by an in-house made software using MATLAB which can conduct the image acquisition and processing, computer vision, an instrument control, and Arduino support toolboxes for execution. For example, to allow the feedback system, this can involve configuring three parts of the software: (1) DMF grid configuration, (2) sequence generation, and (3) feedback and analysis setup. In the DMF grid configuration, users can create their own designs that match their device design by entering a grid specifying the number of rows and columns and selecting the squares on the grid to match the user device design. Next, the user will input the “electrode number” matching to the connection on the pogo pin board and switch.

The resulting DMF design grid can be saved for future use in this example. In sequence generation, users have the capability to allow real-time control (e.g., on-demand actuation) or sequence-activated control (e.g., users create their own sequences). For real-time control, users can click on the electrode to allow real-time application of the electric potential to the electrode. For sequence-activated control, users can create a sequence by clicking on the electrode button and save the selection with the “space” key. This can be repeated, saved for future use, and activated when the user is ready for actuation. For either actuation method, users will enter values for voltage, time, and frequency which are parameters required to actuate the droplets on the device.

In the feedback and analysis setup in this example, a variety of parameters can be used with the feedback system. Briefly, users can create a visual grid that is used for storing the coordinates of the electrodes. Users will enter values for electrode size (in pixels), radius size (e.g., typically half of electrode size), detection box (e.g., region of detection), base time (e.g., time duration for one pulse), correction time (e.g., time duration for one correction), base voltage (e.g., initial voltage applied to the electrode), and jolt voltage (e.g., incremental voltage). Using this system, images were acquired and analyzed to check if the droplet is on the destination electrode. In addition, a program was created to acquire images of the droplets that will automatically calculate the pixelated RGB channel values for biological analysis.

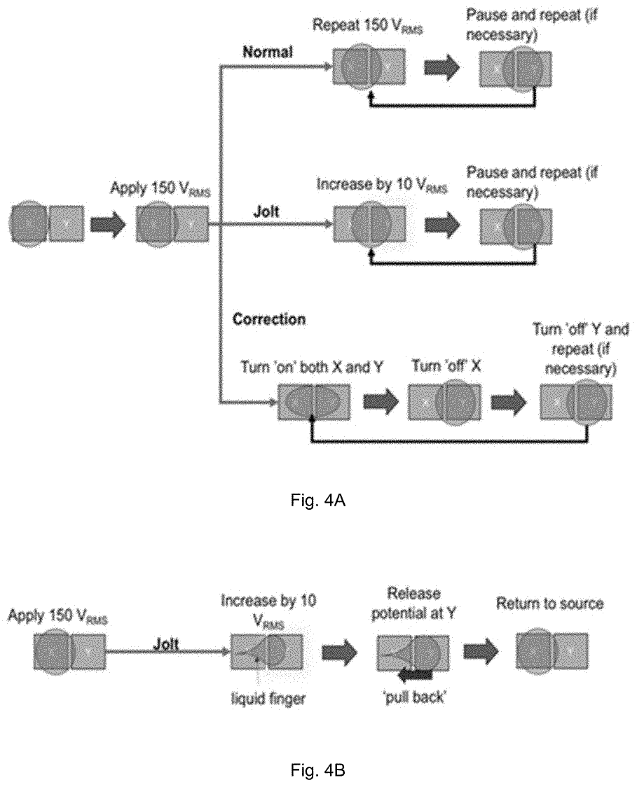

To illustrate droplet dispensing and movement in this example, in a system where there is no feedback, droplet dispensing was initiated by the application of an electric potential (˜150 VRMS; 10 kHz) to a reservoir electrode; then iteratively applied to three adjacent electrodes to stretch out the liquid from the reservoir. To “dispense” the droplet, potentials were simultaneously applied to both the reservoir and the third adjacent electrode. Similarly, droplet movement was initiated by applying potentials to a desired electrode and iteratively applied to adjacent electrodes. This allowed the user to program the number of droplet movements (ND) and record the number of successful droplet movements. To evaluate the feedback system, four actuation schemes was tested to determine the fidelity of droplet manipulation: (1) normal, (2) jolt, (3) correction, and (4) jolt and correction (FIG. 4A).

In the normal scheme, a re-application of the reference potential is applied to the destination electrode (Y) if there is a failure in droplet movement. In the jolt scheme, the destination electrode (Y) was re-actuated with a higher potential in increments set by the user (e.g., jolt voltage) during the setup of the feedback system. If droplet movement does not proceed to Y, this process is repeated until the voltage reaches a limit of 250 VRMS. In the correction scheme, two electrodes—the source (X) and destination (Y)—were actuated with the same applied voltage. If there is a droplet movement failure, the scheme would (1) actuate both X and Y electrodes for a user-specified duration (e.g., the correction time) and (2) turn off electrode X, while leaving electrode Y on for an additional correction time. In the jolt and correction combination scheme, the program will start with the correction scheme and increase the voltage on electrode Y (by the jolt voltage) at the end of the correction scheme. For these schemes, the droplet velocities were measured for each movement, which is the ratio between the size of an electrode (D) and the base time set by the user of one pulse (TD) (e.g., V=D/TD).

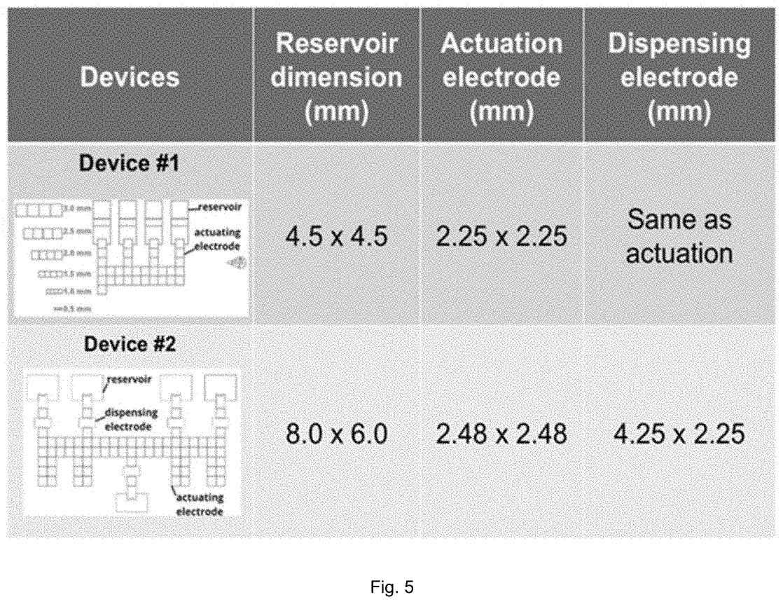

In feedback mode, dispensing and movement followed a similar process with an additional time used for analyzing the images (TI). The time for checking the images was typically −500 ms. Hence, the droplet velocities were calculated as V=D*ND/(NA×(TI+TD)) where NA is the number of electrode actuations. For example, the experiments can be conducted on the devices shown in FIG. 5. For example, experiments were conducted with device 1 (see FIG. 5) with a gap height of 70 micrometer.

Referring to FIG. 4A, a schematic shows the actuation schemes tested with the imaging feedback system in this example. In the normal scheme, an additional 150 VRMS potential was applied to the destination without increasing the voltage. In the jolt scheme, the voltage was increased by 10 VRMS (or set by the user) for each actuation cycle. In the correction scheme, both source and destination electrodes were activated at the reference potential of 150 VRMS.

The combination of jolt and correction (not shown) was tested which starts with the correction scheme and then increases the reference voltage (150 VRMS) by 10 VRMS to the destination electrode Y at the end of the correction scheme (not shown). Referring to FIG. 4B, a schematic shows the “pull-back” problem frequently demonstrated using the jolt scheme with highly viscous biological liquids.

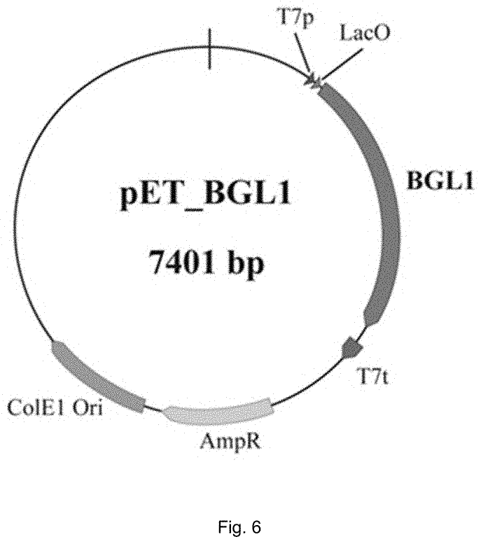

This example was also used in a beta-glucosidase enzymatic assay. Referring to FIG. 6, there is shown a plasmid map of pET_BGL1 of a pET16b backbone with BGL1. Other parts in this plasmid included a T7 promoter and terminator with ColE1 origin of replication and ampicillin resistance.

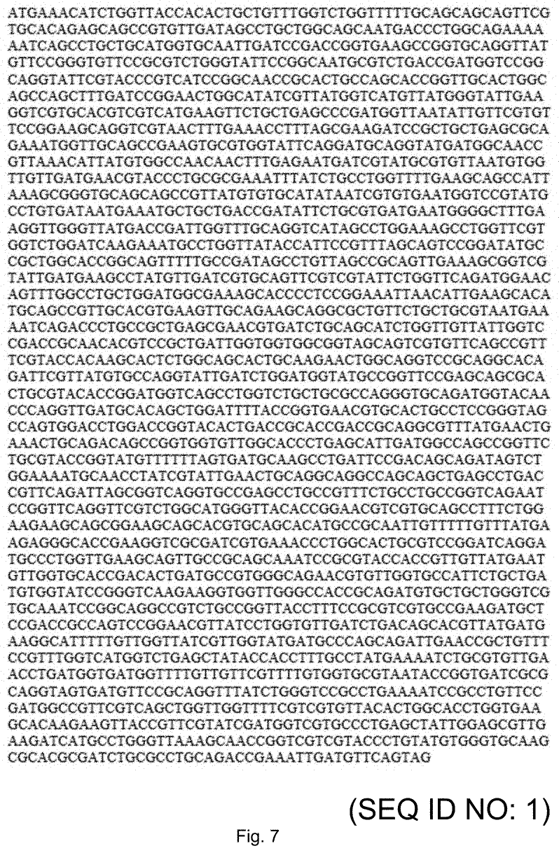

The assay on-chip included three different solutions loaded onto the DMF device reservoirs. First, a unit droplet of cell lysate was dispensed and actuated to each of the four assay mixing areas (see FIG. 5 for DMF design) using a starting voltage of 230 VRMS at 15 kHz. The lysate was prepared from a colony of BL21(DE3) transformed with a plasmid containing beta-glucosidase (BGL) gene (see FIG. 6 and FIG. 7 for plasmid map and sequence (SEQ ID NO: 1) respectively) that was grown at 37° C. and induced at 0.4 O.D (˜1.75 h starting at 0.1 O.D). The assay started by the addition of a droplet containing substrate to a droplet of cell lysate. The substrate solution contained 50 mM sodium citrate at pH 7.0 and 4 mM 4-nitrophenyl beta-D-glucopyranoside (MUG). The reactions were incubated at different times (0, 40, 80, and 120 min) and arrested by the addition of a unit droplet of 0.3 M Glycine-NaOH on the assay areas on the device. Solutions contained 0.05% final concentration of F-68 Pluronics. Three replicate trials using three different devices with gap heights of 280 micrometers were conducted with feedback control. The blue color channel pixel intensity of the droplet was acquired using the imaging-feedback system after addition of the glycine-NaOH droplet and plotted over time.

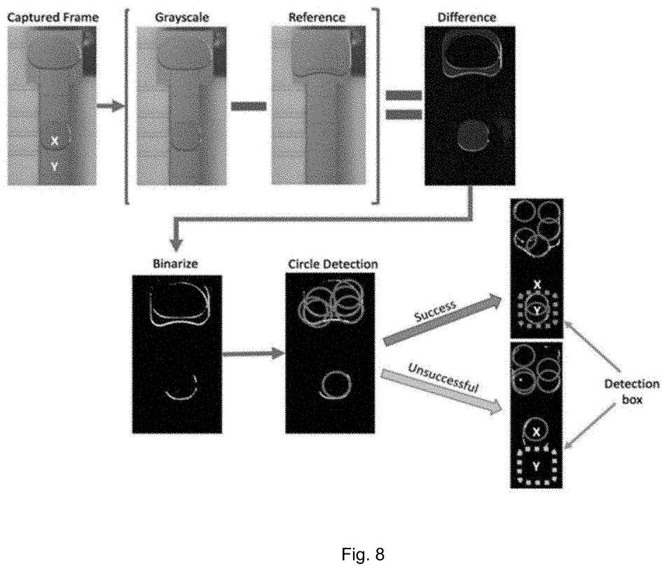

This example also included an image-based feedback system. Referring to FIG. 8, there is disclosed an algorithm of the image-based feedback system. As shown, a droplet is resting on the x electrode and the automation system applies potential to the y electrode. A frame is captured after an actuation. A difference frame is created by taking the difference from a grayscale image and a reference image (e.g., no dispensed droplets). A binarized frame is created from the difference frame. From this frame, a Hough transform allows the detection of circles and returns a successful or unsuccessful result depending on the location of the actuated droplet and the user-defined detection box.

A custom MATLAB program (Mathworks, Natick, Mass.) can be written to implement the new imaging and analysis feedback system. To setup the feedback system, a reference image was acquired with no visible droplets on the electrode path except on the reservoirs. This reference image is acquired for edge detection of the droplet and subtraction techniques for droplet detection. To detect the droplet position, four operations were executed every 500 ms to determine if the droplet dispensed from the reservoir, or moved successfully onto the destination electrode (FIG. 8). The destination electrode is any electrode (e.g., a reservoir or actuation electrode) that has an applied potential. Operation (1) acquires a capture frame that shows the droplet on the source (shown as “x”) and the destination (shown as “y”) electrode. Operation (2) calculates a difference image by subtracting a reference image (taken from setup) from a grayscale image such that it identifies the droplet boundary. Operation (3) binarizes the difference image (e.g., digitizing the image to 1's and 0's) which is to intensify the faint droplet boundaries to stronger ones similar to intensity thresholding or maximum computation.

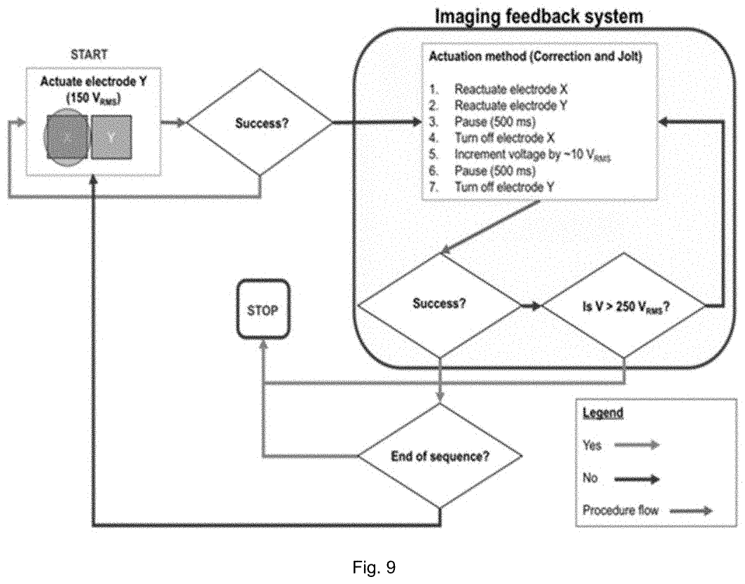

Operation (4) uses a Hough Transform to detect the circles (e.g., the shape of droplet) at the destination electrode and returns a successful or unsuccessful result. An unsuccessful droplet movement would allow the program to start one of the four actuation schemes (described herein) to the destination electrode “y,” while a successful droplet movement continues to the next droplet movement event in the sequence. Since two electrodes were actuated simultaneously (reservoir and the third adjacent electrode) for dispensing, only the actuation (not the reservoir) was considered for detecting dispensed droplets. A control logic flowchart showing the feedback and analysis steps is presented FIG. 9.

Referring to FIG. 9, a flowchart is shown, summarizing the algorithm used to manage the image-based feedback system according to one example. Droplets are actuated with a 150 VRMS AC signal with 15 kHz. The imaging feedback system is initiated if the droplet does not move to the destination electrode (shown as Y). The actuation method is a feedback scheme to move the droplet onto Y (see methods). As an example, the schematic shows the procedure for the jolt and correction actuation scheme. This can be switched to only jolt or correction depending on the user selection at the beginning of the program setup. If droplet movement failed, the algorithm will continue with the actuation scheme until the voltage surpasses 250 VRMS or if the droplet has moved to the electrode Y. If the droplet movement is successful, the algorithm continues with the droplet movement sequence unless the sequence is finished.

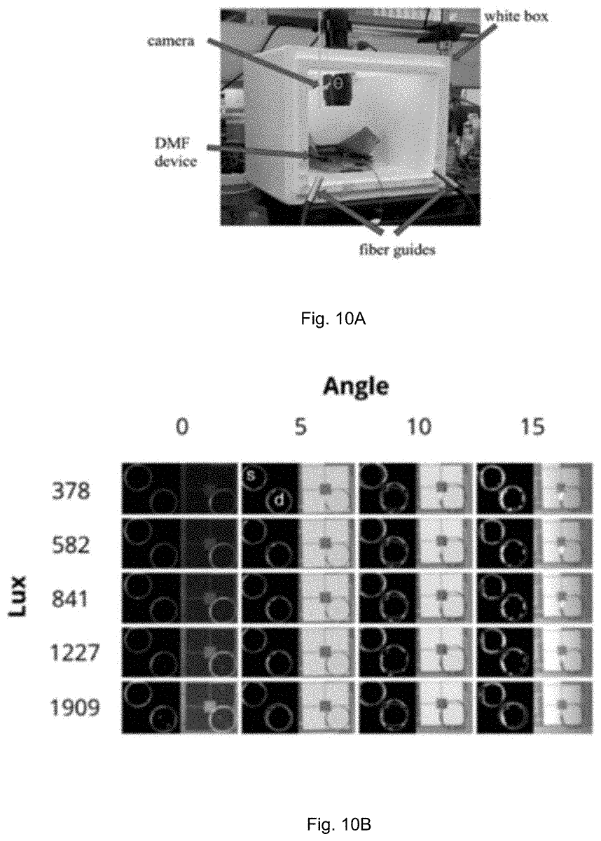

To characterize the feedback system of this example, FIG. 10A shows a setup of a camera with the measured angle surrounded with a white backdrop. FIG. 10B illustrates a set of images showing the success of droplet detection as a function of camera angle (°) at different light intensities (lux). A droplet was placed at a source electrode (labelled as s) and were actuated to a destination electrode (labelled as d) to determine if the image software can detect the droplet. Two images (circle detection—left and original—right) were shown for each angle and light intensity.

In initial experiments, it was observed that the droplet detection efficacy using the imaging software was not uniform on different regions on the device (e.g., ˜40% droplets were detected). This could be due to the lighting from the environment and the alignment of the camera with respect to the device, which can induce false positive or negatives. To mitigate this, an external backdrop was designed (see FIG. 10A) that maintains uniform lighting around the device. This external backdrop used a white-colored box with a dual-branch fiber optic illuminator to guide the light into the box. After this modification, the lighting system was characterized by examining the lighting intensity and the alignment of the camera and determining its effect on droplet detection using the detection software (see FIG. 10B). In these experiments, a series of test images was collected, containing a droplet at a reference electrode and moving it to an adjacent electrode. Based on the results, no errors in droplet detection were observed at the angles and light intensities tested, demonstrating the efficacy of the imaging algorithm. Although high success of detection was obtained, a camera angle of 5° was chosen since optimal contrast between the droplet and the electrodes on the device was obtained.

Next, experiments were performed to assess the impact of the radius size parameter and the size of the electrode on droplet detection. Here, device number 1 was used (see FIG. 5) containing different sized electrodes and the box size of detection was systematically changed to determine if the droplet can be detected by the imaging feedback system. Electrode sizes of 1, 1.5, 2, 2.5, and 3 mm were used, holding liquid volumes of 70, 157.5, 280, 437.5, and 630 nL (for a 70 micrometer spacer) respectively covering the area of the electrode. For each electrode size, the size of the detection box (in pixels) was systematically changed and then the image detection software was executed to determine if the droplet is successfully detected. This is an important feature in this example program to ensure a range of droplet volumes can be detected, especially in cases where droplets are merged together.

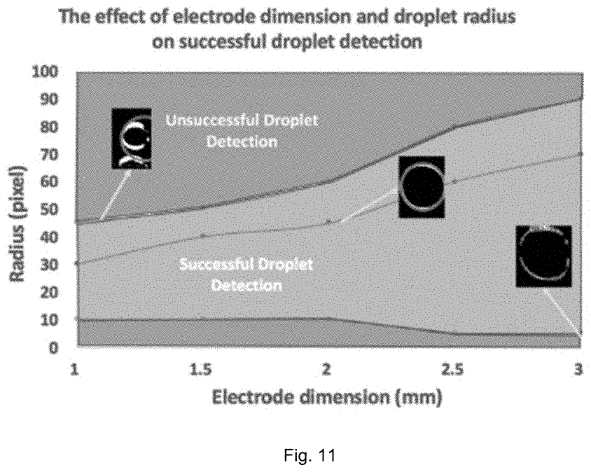

FIG. 11 shows the effect of electrode dimension and droplet radius on droplet detection. A smaller electrode dimension (1 mm) has a smaller range of successful droplet detection compared to a larger electrode dimension (3 mm). Insets in the graph show image views of a successful droplet detection. The middle line is showing the case when a radius that is half of the electrode size is used.

As shown in FIG. 11, a smaller electrode dimension (e.g., 1 mm), as used in this example, has a smaller range for successful droplet detection compared to a larger electrode dimension (e.g., 3 mm). False positives (i.e., droplets are ‘detected’ when there is not droplet present) or negatives (i.e., droplets are present and not detected) can be avoided if the detection box size is chosen within the upper and lower limits (e.g., shown in the shaded region). The ideal detection box size is one-half of the electrode size since 100% successful droplet detection was obtained.

After sensing the droplet position, the feedback system in this example was programmed to repeat the application of an electric potential onto the destination electrode. However, frequent failures were observed after detection using this typical scheme especially for liquids that have proteins (˜10% of the 50 programmed movements were successful). Therefore, this allowed assessing different actuation schemes by calculating the number of completed droplet movement steps and the number of feedback actuations required after a failure is encountered.

In this example, multiple actuation schemes that can be used to move droplets that resist movement were assessed. Three different schemes were tested: jolt, correction, and jolt and correction (as described above) and compared it to a normal scheme (i.e., reapplication of the potential of same magnitude) using complete cell media of RPMI 1640 with 10% FBS. Other types of liquids were not tested since feedback-sensing is typically not used for liquids without proteins as per observation (see below) and shown from other studies. In Table 1 below, the jolt scheme temporarily increased the electric potential by 10 VRMS each cycle and is successful in moving the droplet ˜16% of the time.

TABLE 1

| | | | Average |
|---|---|---|---|
| | Completed | Total no. of | feedback |
| | actuations | feedback actuations | actuation |
| Actuation schemes | out of 50 | per 50 steps | per step |
| Control | 4.7 ± 1.8 | 17 | 4 |
| Jolt | 8 ± 2.8 | 16 | 2 |
| Correction | 50 | 8.1 | 0.162 ≈ 1 |
| Jolt + correction | 50 | 3.6 | 0.072 ≈ 1 |

However, this actuation scheme frequently would compromise the dielectric causing electrolysis at high voltages which renders this particular example device useless. Furthermore, the increase in electric potential induced droplets to move to the destination electrode but frequently would “pull back” to the source electrode after applying the increased voltage on the destination electrode (FIG. 4B). A different switching scheme may alleviate this “pull-back” problem-specifically, turning on both the source and the destination electrode will allow overlap with the destination electrode while preventing any “pull-back” of the droplet to the source. The data validated the hypothesis—a significant increase in successful droplet movement was observed after the correction actuation scheme is initiated compared to the jolt scheme—16 vs. 100%. In most cases when the initial droplet movement has failed, generally only one correction actuation was required while two jolt actuations were required per failed droplet movement due to the pull-back problem.

For completion, the combination of the jolt and correction was tested and similar success completion rates (100%) were observed as to using only the correction scheme. On average, only one jolt and correction actuation were typically required since the jolt was used in combination with the correction. This suggested that the correction scheme with feedback is most favorable for moving liquids that is similar in viscosity to complete cell media on DMF devices since it prevents the “pull-back” problem and avoids any degradation to the dielectric.

Droplet dispensing is an operation commonly conducted on digital microfluidic devices. Dispensing is defined as a success if the dispensing protocol produced a unit droplet with user specified volume. Several studies have examined the droplet dispensing and have characterized the mechanism of droplet dispensing. These studies investigated the variation in volume of dispensed droplets and correct the variation of the volume by capacitive sensing and feedback control. Unfortunately, these systems mainly focused on repetitive dispensing of droplets from a reservoir—e.g., serially dispensing one droplet during a sequence—and studying variation in the volume of dispensed droplets. A drawback with their systems is that they are not capable of detecting individual dispensing failures, only detecting if there is a variation in volume present.

This example illustrates the harnessing of digital microfluidics to be able to do multiplex dispensing during one sequence, e.g., parallel dispensing of droplets. Application of the imaging feedback control to multiplexed dispensing may allow detection of individual dispensing failures as shown in this example.

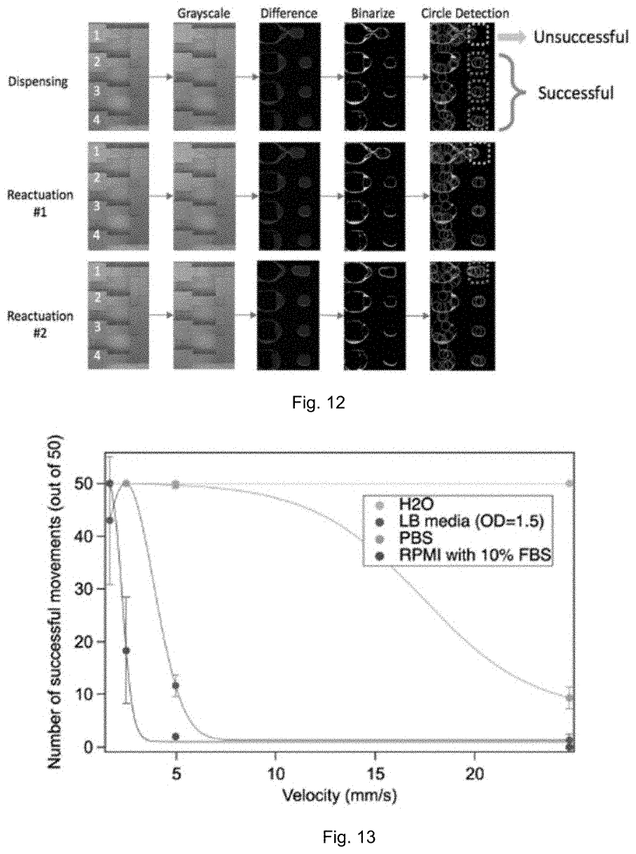

Referring to FIG. 12, there is illustrated a multiplex dispensing showing detection of a single droplet dispensing failure for this example. Rows 1 to 4 are dispensed simultaneously. Rows 2 to 4 show dispensing success while a failure in row 1 is observed. Two additional applications of potentials (#1 and #2) are only applied to row 1 while droplet on rows 2-4 continue with the program sequence.

As shown in FIG. 12, three droplets containing water and one droplet containing LB (lysogeny broth) media were dispensed in parallel following the typical actuation procedure for dispensing (described in methods). In rows 2-4, dispensing was a success as droplets were observed within the detection box (i.e., destination electrode) while in row 1, dispensing failed and required sensing and feedback to complete the droplet dispensing process. Three replicate trials were conducted and each trial showed the droplet dispensing protocol regularly failing to produce a unit droplet with an initial application of electric potential for viscous liquids, especially for liquids containing proteins (e.g., LB media). This suggests that there is a need for sensing and feedback for dispensing liquids containing proteins. For example, individual detection of dispensed droplets becomes important for biological assays as it reapplies electric potentials to only failed droplet movements without excess application to electrodes with successful droplet movements. This may minimize biofouling since more actuations reduces the contact angle of the droplet. Furthermore, excess actuations may increase degradation of the dielectric layer which may reduce the lifetime of the device.

In addition to droplet dispensing, the image-based feedback system of this example was also validated by evaluating droplet movement for four liquids that are commonly used in biological assays: DI water, PBS, LB media with E. coli (at O.D. 1.5), and RPMI with 10% FBS.

In the tests, droplets were actuated across a linear device having 10 electrodes and were repeated five times giving rise to a total of 50 movements. Actuation base times was changed (TD—100, 500, 1000, 1500 ms) and the number of successful droplet movements out of 50 steps was measured.

Referring to FIG. 13, there is shown the effect of droplet movement on DMF devices without feedback. Four liquids: DI water, PBS, RPMI with 10% FBS (complete cell media), and LB media (with O.D=1.5) were tested on 10 electrodes at different velocities (i.e., at different base times of 100, 500, 1000, and 1500 ms) and were repeated five times to give a total of 50 actuations. The error bars are +/−one standard deviation from three replicate trials. Table 2 illustrates a table showing the velocities of liquids with feedback.

TABLE 2

| | No. of | | Avg. | Avg. |
|---|---|---|---|---|
| | successful | Base Time | Total Time | Velocity |
| Liquids | movements | (ms) | (s) | (mm/s) |
| Water | 50 | 100 | 6.10 | 18.66 |
| | 50 | 500 | 26.30 | 4.28 |
| | 50 | 1000 | 53.83 | 2.11 |
| | 50 | 1500 | 78.77 | 1.43 |
| PBS | 50 | 100 | 48.31 | 2.71 |
| | 50 | 500 | 50.08 | 2.47 |
| | 50 | 1000 | 74.04 | 1.67 |
| | 50 | 1500 | 99.83 | 1.24 |
| RPMI | 50 | 100 | 73.61 | 1.69 |
| | 50 | 500 | 51.50 | 2.41 |
| | 50 | 1000 | 80.39 | 1.54 |
| | 50 | 1500 | 134.60 | 0.95 |
| LB | 50 | 100 | 64.06 | 1.93 |
| | 50 | 500 | 49.18 | 2.52 |
| | 50 | 1000 | 74.31 | 1.67 |
| | 50 | 1500 | 101.30 | 1.22 |

As shown in FIG. 13, the number of successful movements is highly dependent on TD. Specifically, with a single application of an electric potential with no feedback, higher velocities (or fast base times: 100 or 500 ms) generally results in poor droplet movement for non-water liquids. Furthermore, there is high variability of success for liquids that contain proteins (e.g., RPMI with 10% FBS and LB media with E. coli) at slower velocities (1.65 mm/s and 2.48 mm/s) due to the heterogeneous mixture of the solution. This is problematic for digital microfluidics as the droplet transportation efficiency is highly variable for protein-rich liquids at low velocities (<5 mm/s) and therefore depends on chance for completion. However, with the image-based feedback system improvements in velocities were observed (e.g., faster for the droplet to reach the destination) and more importantly an increase in the number of successful droplet movements was observed. As shown in Table 2, perfect droplet movement fidelity was obtained (out of 50 movements) with average velocities of ˜2.5 mm/s and 2-3× increase in velocities (compared to no feedback) for protein-rich liquids. In addition, fast base times of 100 ms are favorable for moving droplets containing no proteins (e.g., PBS and H₂O) while 500 ms are favorable for protein-rich liquids (e.g. RPMI with FBS and LB media). This is a similar observation compared to a previous study where the fast base times are not enough to account for the viscosity of the liquid and slow base times are exacerbating surface fouling. Therefore, this shows that there is a need for an image-based feedback system for moving protein-rich liquids that will automatically optimize the base times to move these types of liquids.

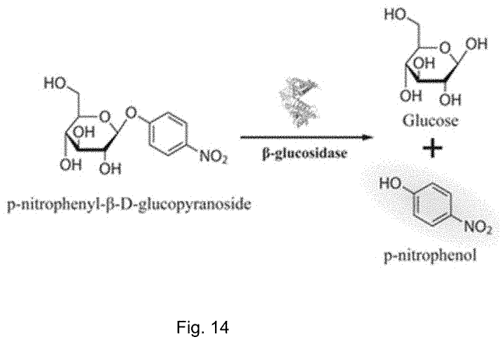

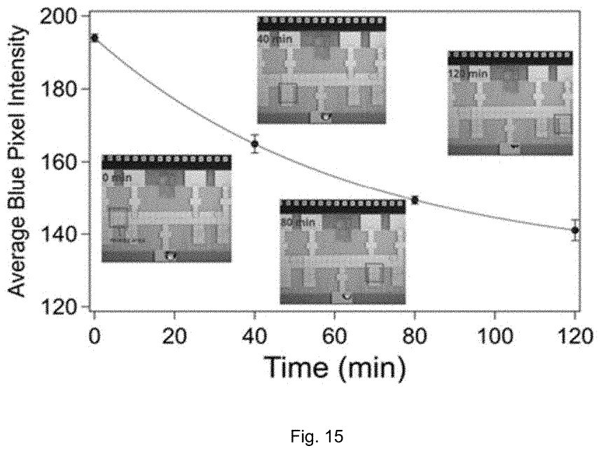

This example was also used in a beta-glucosidase enzymatic assay. Referring to FIG. 14, there is shown a chemical scheme of the enzymatic assay. Referring to FIG. 15, there is shown a curve depicting the average blue channel pixel intensity as a function of time. The average blue channel pixel intensity was collected every 40 min intervals on device #2 with the image-based feedback system. Inset shows series of frames at the different time intervals depicting the enzyme assay and where the droplets were analyzed (box). Each experiment was repeated in triplicate on separate devices, and error bars are +/−SD.

To demonstrate the applicability of the image-based feedback system, the activity of a beta-glucosidase enzyme that is useful for the production of biofuels was examined. Cellulose has great potential as a renewable energy source and the enzymatic hydrolysis which is completed by beta-glucosidases is a promising green alternative for the production of fuels. The typical model for analyzing kinetics of a beta-glucosidase enzyme is to use a chromogenic model substrate para-nitrophenyl-beta-glucoside (pNPG) that will produce glucose and para-nitrophenol upon hydrolysis (FIG. 14). The liberation of para-nitrophenol (pNP) gives a yellow color product which can be monitored by an image-based feedback system.

Some groups have incorporated image-processing techniques on droplets by capturing an image and using it, either as a threshold value for intensity or comparing the image captured from a video with a standard image. For the kinetics analysis, a different approach was used to measure the activity of the enzyme. Using device #2 (see FIG. 5), an automated feedback system was used to dispense and to move the substrate and lysate to the mixing and detection areas on the device and calculated the RGB profile for a region of interest (ROI) inside the droplet without any external optical detectors (e.g., well-plate reader or optical fibers) at different time intervals (FIG. 15). Using the MATLAB program colour_analysis.m, a ROI that is covering 25% of the droplet was selected and the pixel intensities were averaged for each color channel: red, green, and blue. As expected, the red and green channels did not show any significant difference in the pixel analysis of the pNP yellow product (data not shown). From the blue channel, as shown in FIG. 15, the graph depicts the change in yellow color as a function of time showing differences in blue channel pixel intensities for the pNP product in reaction droplets that were mixed with feedback control. In initial experiments without feedback, moving and dispensing droplets containing the lysate and the substrate were difficult due to large gap heights (˜280 micrometers) which caused the experiment to fail over 95% of the time. However, with the feedback system, droplets were dispensed with >99% success rate while moving droplets to the destination electrode with perfect fidelity. Additionally, the droplets were merged and this droplet was detected with the same fidelity. This high success rate may be due to the capability of the feedback system to correct individual droplet operation failures while concurrently actuating droplets that were successful in movement to the destination. Using the image-based feedback approach allowed for moving and dispensing protein-rich liquids and analyzing the product of an enzymatic assay.

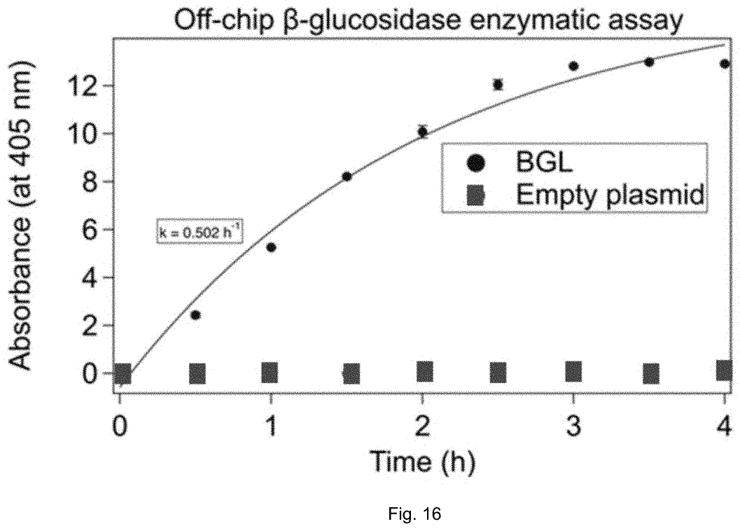

In the same experiments, it was possible to extract first order rate constants and compared it to off-chip reactions. The extracted value generated from the image-based feedback system is kDMF=0.167 h⁻¹ and the rate constant from off-chip experiments is kPLATE=0.504 h-1 (FIG. 16). It was noted that there are some differences in the rate constants since different pieces of optical equipment were used (camera vs. well-plate reader) to analyze the pNP product.

Integration of lenses and filters to the camera setup may be able to give a closer estimate to the well-plate reaction rate constant. Nevertheless, it is proposed that discovering relative activities between enzymes or any applications requiring automated mixing of protein-rich liquids will be highly suitable for the image-based feedback system.

Referring to FIG. 16, there is shown off-chip enzymatic assay with an absorbance readout as a function of time were collected every 30 min. Nine reactions containing equal volume of lysate with enzyme and substrate were mixed in a well of a 96 well plate. At 30 min intervals, the reaction was arrested by glycine-NaOH solution and an absorbance measurement was acquired from the product formation of pNP which gave a yellow color. Each experiment was repeated three times, and error bars are +/−1 SD.

This example demonstrates an automated image-based feedback system to move and to dispense biological fluids on digital microfluidic devices. The image-based feedback system uses a reference and subtracting technique with a Hough transform to visualize the droplets on the device. The image-based feedback system was characterized and the optimal camera angle, lighting intensity, radius of detection, and correction method to implement for high success of droplet detection were determined. Furthermore, this system is capable of detecting individual droplet dispensing and movement failures and implementing feedback while concurrently continuing with other droplet operations on the device. To show the utility of the system, it is used to conduct an enzymatic assay that uses the image-based algorithm to analyze the enzymatic product without requiring any other external detectors. The image-based feedback and analysis system is an automated solution for multiplexed biological assays whose performance exceeds other technologies on the market.

### Example 2

This example illustrates an automated induction microfluidics system for synthetic biology, in accordance with certain embodiments of the invention.

Synthetic biology has emerged as a way to create a useful biological system for various applications. Building such biological systems can be an extensive operation and often through trial-and-error processes. A process commonly used in synthetic biology is induction. Induction uses a chemical inducer IPTG to express high levels of a protein of interest. The conventional protocol remains broadly used despite requiring to frequently check the density of a growing culture over several hours before manually adding IPTG. Here, this example illustrates an automation induction system that was developed for synthetic biology using digital microfluidics without the frequent monitoring of cultures.

Synthetic biology uses a design/test/build workflow to engineer new biological systems. Progress in designing novel biological systems has been hindered primarily by the lack of physical automation systems to expedite this engineering cycle. However, recent advances in automation have allowed to increase the speed and throughput of the process. A promising technology, namely digital microfluidics (DMF), have shown promising results in automating synthetic biology, with common experiments like DNA assembly being automated without manual intervention. A common step in synthetic biology is induction, which uses a synthetic molecule IPTG to induce high expression of a protein of interest in host bacterium E. coli. This protocol requires to manually check the optical density (OD) of the growing culture to determine the optimal time to induce expression. Despite the time and attention required, the conventional protocol is favored to more recent auto-induction media that are able to induce expression alone. As an alternative, automating the OD measurement on a bacterial culture and addition of IPTG would offer convenience for researchers to carry out effortless induction of their cultures. This example shows that the creation of a DMF-based platform for the automated induction of protein expression is reported. The system, called the AIMS, is capable of monitoring the OD of a bacterial culture in order to induce protein expression at the desired time; and to carry out enzymatic assays to assess protein expression.

The DMF devices were fabricated by photolithography. A 7 micrometer layer of Parylene-C was deposited as a dielectric and the devices were coated with hydrophobic Fluoropel PFC1601V before use.

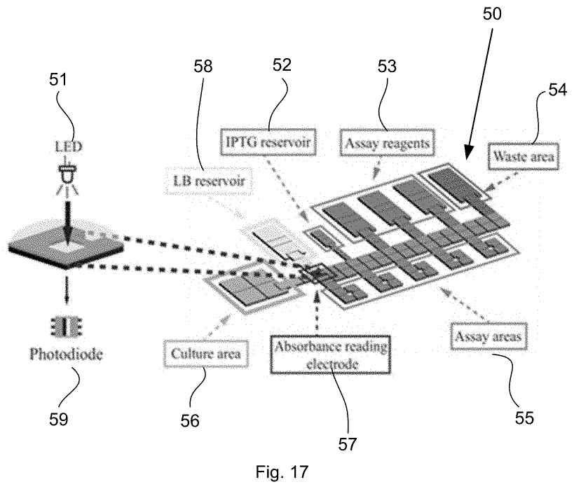

Referring to FIG. 17, there is shown a layout of the automated induction microfluidic system (AIMS) device according to one example. For example, the device can include areas for bacterial culture, incubation, and dispensing reagents. The alignment between the LED and the light sensor allows absorbance readings through on-chip samples of droplets.

Referring to FIG. 17, the device includes a LB reservoir 51, an IPTG reservoir 52, assay reagent reservoir 53, waste area 54, assay areas 55, an absorbance-reading electrode 57, and a culture area 56. For example, on the absorbance reading area, there is a LED 58 on top of the reading electrode; there is a photodiode 59 at the bottom of the electrode for sensing and reading the optical density (OD) and/or absorbance of the material (or droplet) on the reading electrode. The alignment between the LED and the light sensor allows absorbance readings through on-chip samples of droplets.

For example, the DMF design 50 contains an area dedicated to the mixing of a bacterial culture, an incubation area, and 6 reservoirs for dispensing reagents (see FIG. 17). For example, an absorbance window was integrated as a transparent-section in the center of the absorbance-reading electrode. For example, the complete system integrates a 600 nm emitting LED placed above the absorbance window and a light sensor aligned for reading the intensity of the light passing through the sample.

For induction experiments, an overnight culture of E. coli was diluted to OD 0.1 in LB media containing 0.05% Pluronics F-68 surfactant. 20 microliters were placed into the culture area of the chip (FIG. 17). The culture was grown by placing the closed setup in a 37° C. incubator until it reached an OD of 0.4. This threshold OD triggered induction of five daughter droplets with decreasing IPTG concentrations. The induced droplets were left to incubate in the five assay areas (FIG. 17) for four hours before analysis.

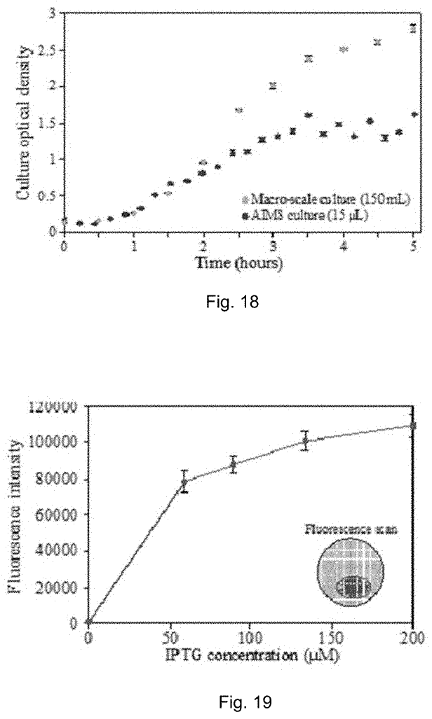

FIG. 18 illustrates a comparison of bacterial growth on the AIMS with a macro-scale culture. The macro-scale culture was generated manually and the micro-scale culture was automated on the AIMS with mixing and optical density (OD) readings.

The ability of the AIMS to accurately read optical density can be validated by generating a standard curve using dilutions of a culture of known OD and automating readings on the system (data not shown). Then, a growth curve was generated by following the OD of a culture mixed on device over five hours (FIG. 18). For comparison, a growth curve was also created from manual OD readings on a macro-scale culture. The AIMS was able to follow OD increase over time with a trend similar to the macro-scale. The micro-scale culture reached a lower final density, as previously observed on small-scale bacterial cultures.

The AIMS is also able to induce the culture upon reaching a certain density. This was demonstrated by inducing a red fluorescent protein (RFP) gene inserted in a pET16b plasmid. In this experiment, individual droplets were mixed and split after induction to obtain four different IPTG concentrations and a droplet of non-induced culture. Automated induction was successful, with the induced droplets showing increased levels of fluorescence relative to the non-induced droplet (FIG. 19). FIG. 19 shows automated induction using the AIMS according to one example. Cultures were grown and induced with decreasing IPTG concentrations and droplets were scanned for RFP expression.

In this example, a system was created for the automation of bacterial culture, induction, and subsequent enzyme assay using DMF technology. This process is automated with on-chip optical density (OD) readings on a growing culture and induction is automatically triggered at a threshold OD. This will allow the AIMS to carry out automated growth, induction, and analysis to facilitate the induction process for synthetic biologists.

### Example 3

This example illustrates an automated induction microfluidic system that will provide a new automated tool to quickly find conditions that are suitable for protein production.

Almost all (if not all) synthetic biology applications need the need of induction, which is the regulation of gene expression in a presence of a chemical inducer. This is can be useful in the case of strain optimization, which follows a typical iterative engineering workflow of design-build-test-learn (DBTL) to simultaneously study a biological system while producing valuable products (e.g., therapeutic agents for disease or biochemical for green energy. The engineering of viable strains may rely on the characterization of DNA parts to attain optimal protein expression and productivity. Therefore, numerous groups have devoted much time into characterizing which conditions are suitable for protein expression.

The goal of this example is to develop an automated induction microfluidic system that will provide a new automated tool to quickly find conditions that are suitable for protein production. The new method can rely on digital microfluidics for handling and delivery of small volumes of reagents which will be integrated into a benchtop instrument that will control the manipulation of fluids and the analysis of the cells and proteins. This example includes two specific aims: 1) to miniaturize the electronics and detection system into a benchtop instrument (similar in size to a well-plate reader), and 2) to develop devices capable of factorial experiments capable of testing 33 conditions.

The expression of a recombinant gene in a host organism through induction can be an extensively manual and labor-intensive procedure. To expedite this process, this example illustrates an automated induction microfluidics system is described-which is called AIMS. The system uses a benchtop platform that will contain electronics with an integrated absorbance and fluorescence reader to allow the real-time monitoring of samples optical density (OD) coordinated with the semi-continuous mixing of a cell culture on a microfluidic device. A microfluidic device is placed on top of the system, and it is used to culture cells and to measure the OD of the bacterial culture. Animal or other cells could also be used. In addition, this platform provides analysis of regulated protein expression in E. coli without the requirement of standardized well plates.

This system offers great convenience without the user to physically monitor the culture or to manually add inducer at specific times. This system is an automated induction system. It is believed that this platform may be useful for synthetic biology or molecular biology applications that require regulating and analyzing expression of heterologous genes for strain optimization.

Development of an automation system for digital microfluidics (DMF). An image-based automation feedback system that is capable of manipulating and tracking droplets on an array of electrodes to ensure high-fidelity droplet movement was developed. See above. As depicted in FIG. 20A, the hardware uses solid-state relays enclosed in a 3D printed box. This enclosure is connected directly to the device for manipulation of droplets without pumps or tubing. FIG. 20B shows the software interface that allows the user to upload their own device designs, program droplet operations with on/off times for actuations and voltage requirements, track droplet movements using feedback, and visualize current droplet manipulations. The sophistication built in this software and hardware allows the control and tracking of ˜100s of droplets on the microfluidic device in preparation for the automated induction microfluidics system (AIMS).

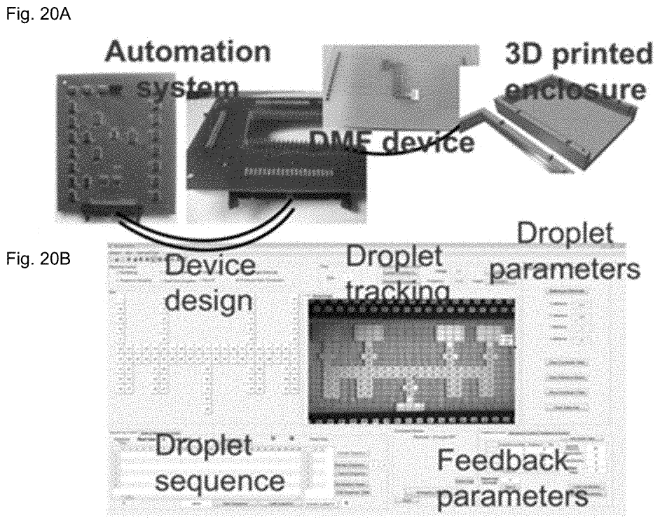

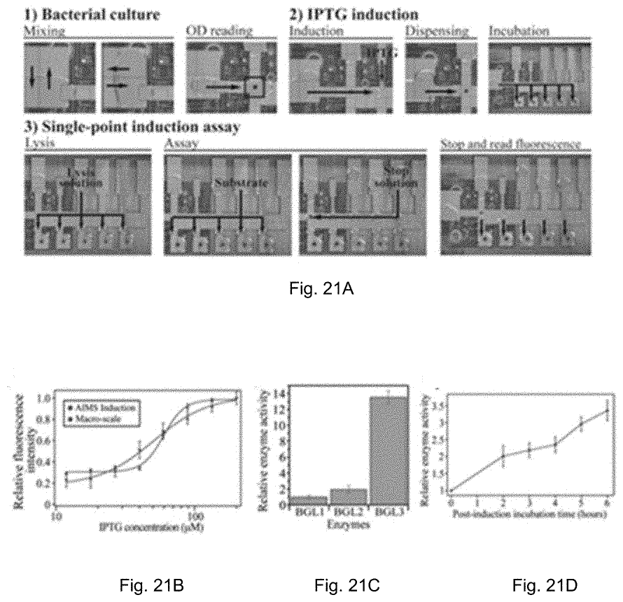

FIG. 21A illustrates images from a movie of an Automated Induction Microfluidic System (AIMS) showing the step of automated culture, induction and protein analysis. FIG. 21B illustrates comparison of dose-response curves of IPTG using AIMS and macroscale cultures. FIG. 21C illustrates comparison of the rates of activity for three enzymes relative to the lowest (BGL1). FIG. 21D illustrates induction profile of the highest activity enzyme over 6 h on the AIMS.

Auto-induction assays on DMF. Using the automation setup described above, auto-induction assays using E. coli cells cultured with different plasmids were implemented on digital-and-channel (“hybrid”) microfluidic devices. The channel part of the device is used to automate the delivery (and refilling) of fluids to the reservoir. The digital part is used to perform the automated culture, induction, and analysis. FIG. 21A shows a sequence of images from a movie depicting the steps of the auto-induction assay from culturing to induction to protein analysis on the device. The system was tested with an IPTG inducible expression vector carrying a red fluorescent protein (RFP) gene downstream of a T7 promoter, although other proteins or vectors could also be used in other embodiments. FIG. 21B shows the similarity in dose-response curves from macro-scale and microfluidics experiments.

To further show the versatility of AIMS, this system was used to test and to analyze conditions suitable for protein expression of a group of enzymes used for breaking down biomass for biofuel production. Depicted in FIG. 21C is a fluorescence intensity curve for the enzymatic assay that was measured directly on the device using an external benchtop scanning well-plate reader. The activity of the most active enzyme was further optimized (i.e. BGL3) to determine the optimal post-induction incubation period for BGL3 expression (i.e. pre-lysis). As shown in FIG. 21D, BGL3 showed higher expression (at least three times higher) after 6 h of induction and incubation compared to immediate induction and lysis (0 h).

Comparison of current vs. state-of-the-art. Current bench scale and robotic technologies do not have the automation and the integration capabilities of the AIMS in this example. The AIMS in this example can automate all of the steps required for protein expression and analysis, driving down costs and increasing speed (see Table 3). The AIMS in this example also enjoys the benefits of low-reagent consumption, automation of cell culturing, induction, and protein expression analysis. The use of digital microfluidics which have salient features, such as droplets, are easily controlled individually (e.g., by application of an electric potential) without the need for channels, pumps, valves, or mechanical mixers, etc., as shown here. All of these various processes are easily performed with a simple and compact design that is affordable to any laboratory.

TABLE 3

| Metric | Manual | Robotics | AIMS |
|---|---|---|---|
| Speed¹ | 1.5 months | 2-3 weeks | ~10 h |
| Equipment | Incubator, shaker, | Robotics, incubator, | Benchtop electronics |
| required | well-plate reader | shaker, well-plate reader | |
| Costs² | >$5-10k | System: >$1 million | System: $10k |
| | | Plates: $6-$10 | Devices: $5-$10 |
| Reagent | 10-50 microliters | 100-200 microliters | 50-1000 nL |
| volumes | | | |
| Footprint | Human | A full laboratory space | A benchtop space |
| Automation | Low - all manual | Moderate - manual | High (fully automated) |
| | processing | protein expression | |
| | | analysis | |
| Multiplexing | No | Yes, but analysis | Yes with all analysis |
| | | requires a plate reader | performed on chip |

¹Time for culturing, inducing and testing 27 conditions.
²Cost estimates for 27 conditions (manual and robotics).

This example shows a miniaturized automated induction microfluidics system for strain optimization and synthetic biology applications. In some cases, the device can be packaged into a benchtop instrument. The device can be made into a benchtop system capable of cell culturing, induction, and analysis. In addition, the device may be capable of automated culture, induction, and/or analysis with identical performance to preliminary results (i.e. 6-fold increase in enzyme activity).

The device may be able to perform, in some cases, factorial testing of conditions for strain optimization. Different tests can be performed on the AIMS in this example. For example, the analysis of 3³ (27) conditions using samples ranging from 100-300 nL to discover enzymes that have >5-fold activity compared to the control can be performed in accordance with one embodiment of the invention.

As mentioned, in some cases, the device can be packaged into a benchtop instrument. This example shows a proof-of-principle system that is capable of culturing, induction, and protein expression analysis using a battery of tests recently designed to determine conditions that are suitable for high enzyme activity. The generation of a low-voltage AC signal with amplification and fluorescence detection were used with offline instruments.

For example, to automate droplet movement on digital microfluidic devices, a function generator and an amplifier may be used. However, these two components are often bulky and are external components connected to the control boards required to activate the electrodes. Thus, in one embodiment, the device may include a sine wave generator and amplifier using FETs that will only take a small footprint of 5″×5″. In some cases, the system may include a microcontroller with a digital-to-analog converter with a low-pass filter to act as a function generator. The output signal from this can be connected to the differential amplifier with current mirrors that will then go through filtering stages to eliminate the high-frequency signals. A simulation was conducted in LTSPICE and showed that the output of this circuit can be a maximum of 400 Vₚₚ (˜140 Vᵣₘₛ) of bandwidth (0.5-20 kHz) which is sufficient for synthetic biology applications (FIG. 22A). FIG. 22A illustrates a simulated output of a proposed circuit.

In another set of embodiments, the device may provide reduced voltages of 100Vₚₚ (˜35 Vᵣₘₛ), reduced bandwidth to 0-1 kHz, a square wave since it only requires rectification with minimal filtering compared to sine wave generation, and/or an IC (instead of FETs) for the amplification stage (e.g., Apex PA94 IC).

Biological and chemical assays typically produce an output that requires detection (e.g., fluorescence). There are useful approaches in which the detector is decoupled from the fluidics (e.g., digital microfluidics coupled with optical plate readers or imaging setups. But these often require external equipment which is not suitable for market purposes. Thus, in certain embodiments, the device includes a miniature setup for detection integrated with the device, e.g., using a LED for excitation source with a manufactured optical fiber connector connected to a photomultiplier tube that can be easily interfaced with the device.

FIG. 22B illustrates a schematic showing the online integration of fluorescence detecting with the AIMS in this example embodiment. As shown in FIG. 22B, an optical fiber connector that can be placed directly below (or above) the device using vacuum will be constructed. For example, there can be a need to reliably collect the fluorescently emitted light from the droplet. The device in some embodiments may allow the fiber optic cable to directly read the output from the droplets using a transparent window to provide 10 pM limit of detection (LOD). In some cases, the device may be able to measure a droplet containing standard solutions of fluorescein to characterize LOD, dynamic range, and sensitivity. In some cases, the device may be able to detect enzyme activity using beta-glucosidase (i.e. BGLs) as a model system. If the LOD is >10 pM, fibers could be designed on the same plane as the device (without vacuum) or using laser light sources (instead of LED), in certain embodiments.

In some cases, the device may be able to automate culture, induction, and analysis with identical performance to preliminary results, e.g., with an at least 6-fold increase in enzyme activity of BGLs tested, and/or with replicate analysis for sample droplets ranging from 100-300 nL volumes.

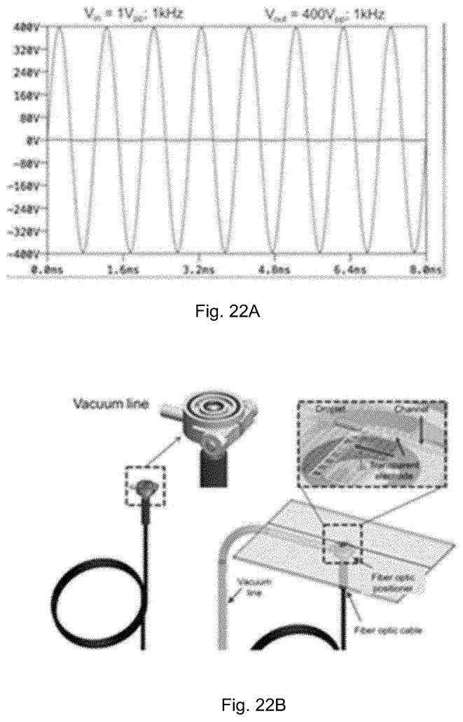

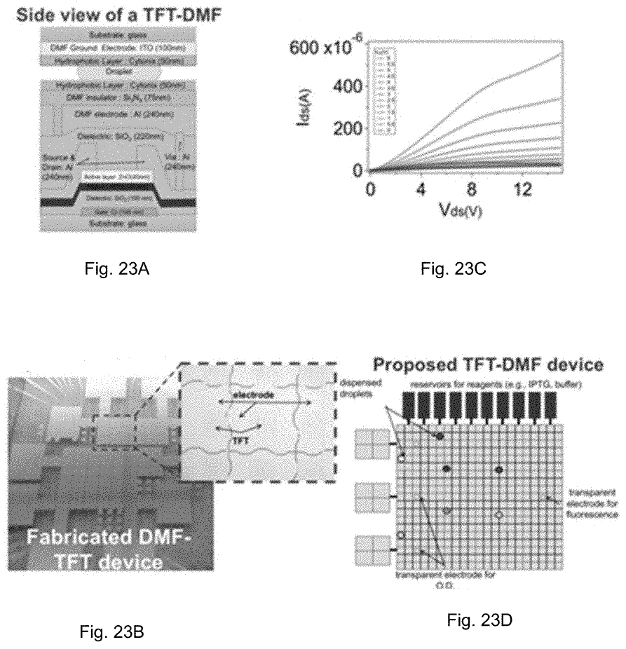

In some cases, the device may be used for factorial testing of conditions for strain optimization. In one embodiment, in anticipation for factorial testing on DMF devices for synthetic biology, a methodology based on active matrix arrays was developed to increase the density of electrodes. As a proof-of-principle, a family of 3×3 active matrix electrodes was fabricated (see FIG. 23A for layers and FIG. 23B for an image of a TFT-DMF device) for automating DNA assembly and transformation (unpublished data).

FIG. 23A illustrates a side view of a TFT-DMF device. FIG. 23B illustrates an image of the fabricated TFT-DMF device. FIG. 23C illustrates a measured I-V curve of 3×3 transistors. FIG. 23D illustrates a schematic of the TFT devices used for factorial experiments.

The electrical properties of the device in this example measured at room temperature and ambient air is presented in FIG. 23C. This platform was also expanded to a 20×20 matrix area such that factorial analysis using the AIMS can performed. As shown in FIG. 23D, there are three culture areas that will lead to an absorbance-reading electrode to monitor the OD in this example. In addition, there were four additional reservoirs that will contain fresh culture media, inducer (i.e. IPTG), and assay reagents (e.g., stop solution and buffer). To show the capabilities of the device, three variables (with three conditions each) that will have an effect on protein expression were tested: inducer concentration (0.25, 0.5, or 1 micromolar), incubation time after induction (4, 6, and 8 h), and OD induction (0.4, 0.5, or 0.6). This will allow 27 different conditions being tested in parallel on the AIMS.

The device can exhibit a driving voltage of the TFT-DMF device of <25 Vᵣₘₛ, a drain current of at least 10⁻⁶ A to ensure TFTs are turned on, and/or an Iₒₙ/I_{off} of >10⁷ such that there is less leakage current and more gate control. In some cases, the driving voltage may be ˜30 Vᵣₘₛ (but not exceed or the device will breakdown) or the Iₒₙ/I_{off} ratio may be at least 10⁶. For example, the drain current can be at 10⁻⁶ A to ensure fully operational transistors.

The device may be useful for analyzing 3³ (27) conditions using samples ranging from 100-300 nL to discover BGL enzymes that have >5-fold activity.

### Example 4

This example illustrates an automated induction microfluidics system (AIMS) for synthetic biology, in accordance with one embodiment of the invention.

The AIMS in this example is a system capable of automating the induction of heterologous gene expression on a digital microfluidics device. The entire process can be automated by AIMS, which includes bacterial cell culture, OD readings, addition of the inducer, incubation, and carrying out an enzymatic assay. Specifically, the AIMS in this example can frequently check the OD of a composition (such as a bacterial culture) being mixed on device. Then, it adds the inducer to the culture such that the operation is carried out upon reaching a certain OD value. After induction, an enzymatic assay (or other biological assays) can be implemented by the successive mixing of several reagents, and analyzed by fluorescence. The present device in this example eliminates the need of manual intervention: monitoring cell culture density, adding inducer, or mixing reagents for enzymatic assays, which are frequent steps required for molecular biologists. It also introduces a reduced experiment scale where reagent use is minimized, and high-throughput multiplexed experiments can be easily included.

The AIMS device presents advantages over marketed auto-induction media in that any induction or protein expression strategy can be implemented, with the added advantage of automation. Applications for the AIMS device in this example are found in synthetic biology, or for any biological experiments that require monitoring of bacterial growth, induction, or testing the activity or expression of various proteins under controlled conditions.

The expression of a recombinant gene in a host organism through induction can be an extensively manual and labor-intensive procedure. Several methods have been developed to simplify the protocol, but none has fully replaced the traditional IPTG-based induction. To simplify this process, the development of an auto-induction platform based on digital microfluidics is described in this example. This system uses a 600 nm LED and a light sensor to allow the real-time monitoring of samples optical density (OD) coordinated with the semi-continuous mixing of a bacterial culture. A hand-held device was designed as a micro-bioreactor to culture cells and to measure the OD of the bacterial culture. In addition, this device can serve as a platform for the analysis of regulated protein expression in E. coli without the requirement of standardized well-plates or pipetting-based platforms.

This example illustrates a system that offers great convenience without the user to physically monitor the culture or to manually add inducer at specific times. The system was characterized by looking at several parameters (electrode designs, gap height, and growth rates) required for an auto-inducible system. As a first step, an automated induction optimization assay was carried out using a RFP reporter gene to identify conditions suitable for the system. Next, the system was used to identify active thermophilic beta-glucosidase enzymes which may be suitable candidates for biomass hydrolysis. Overall, the platform in this example may be useful, for example, for synthetic biology applications that require regulating and analyzing expression of heterologous genes for strain optimization.

Using synthetic biology, several key biological functions can be engineered in living cells to yield valuable products such as therapeutic agents for diseases or biochemicals for green energy. It follows a typical iterative engineering workflow of design-build-test-learn (DBTL) to simultaneously study a biological system while creating these useful technologies through rational design and assembly of DNA from varied sources. While the field of synthetic biology has advanced rapidly in recent years, certain technical challenges still exist like the development of strains due to difficulties in anticipating the combined effect of various DNA parts (e.g., expression constructs) and assay conditions. The engineering of viable strains relies on the characterization of genetic parts to attain optimal protein expression and productivity. As a result, numerous research groups have devoted much time into characterizing DNA parts by screening for their ability to confer improved phenotype. For example, many libraries of promoters (designed via mutagenesis) have been tested to regulate transcription rates and to improve overall protein expression. Additionally, several inducible promoters have been designed in E. coli and in other types of bacteria that allow independent control over the expression of downstream genes. Also, commercially available systems, like the pET expression system, are often used to control the expression of recombinant genes in E. coli. The system in this example uses a T7 promoter controlled by the lac operator that allows gene expression in the presence of an inducer, e.g., IPTG. Using induction for the purpose of strain optimization usually involves growing a culture of cells with the desired exogenous constructs to an optimal optical density (OD), followed by an addition of an inducer. The cells are harvested after growth in the presence of the inducer and tested for the desired output, usually the expression of a protein of choice. In addition to the high costs of inducers, this is a manual and labor-intensive process, requiring frequent optimization of expression conditions, such as inducer concentrations and growth conditions to achieve optimal levels of protein expression. Hence, the need for a more simplified and automated protocol would eliminate the need to constantly monitor cell growth, actively induce expression of the target gene at the appropriate time to obtain a desired level of expression, and/or allow faster screening of parameters affecting recombinant protein expression to rapidly inform iterative strain optimization efforts.

A common practice to automate the expression of genes is to use an auto- or self-inducing system. Auto-inducible systems allow the culture to increase in density before induction of recombinant proteins since these systems are regulated by endogenous or induced metabolic changes during the growth. As opposed to IPTG-based manual induction methods, the auto-inducing systems do not require monitoring of culture density and reduce the chances of contamination. Although improving upon the induction protocol, the auto-induction protocol removes the capability of control, e.g., not knowing the cell density and the relative amounts of nutrient sources to induce protein expression. Inability of control over these factors using auto-induction often produces higher levels of target protein per volume of culture than standard approaches, which could cause a higher metabolic burden and inhibit cell metabolism and growth, which may alter the outcome of protein expression. Furthermore, the auto-inducing system does not optimize or provide analysis of protein expression. Therefore, a technology that allows the flexibility of time and quantity of induction while simultaneously providing automation to monitor cell density and screening/analysis of different parameters that affect recombinant protein expression may be a suitable alternative for controlling and improving protein yields.

Recently, a technology called microfluidics has been developed to miniaturize chemical and biological processes onto hand-held devices. Microfluidics have numerous advantages: reduction in volumes (1000× compared to bench techniques), high-throughput processing, and potential to automate fluidic processes. It has been applied to a host of applications such as cell-based monitoring, point-of-care diagnostics, and synthetic biology. Traditionally, these devices have streams of microliter-volume fluids flowing inside a micron-sized channel. An alternative to microchannels is digital microfluidics (DMF), which uses an array of electrodes fabricated on a chip such that nL (or pL-range) volume droplets can be manipulated on the device. The versatility of DMF allows control over the droplets—e.g., dispensing, splitting, merging, and moving droplet operations—and therefore is a natural fit for automating fluid handling operations related to synthetic biology since it has the capability of integrating and automating the DBTL cycle into a coherent whole.

This example illustrates an automated induction microfluidics system (AIMS) that has been designed for synthetic biology to provide a platform that will optimize and analyze parameters affecting expression of proteins, as a non-limiting example. This description focuses on three components: (1) a DMF platform to culture and to induce biological cells and to analyze protein expression, (2) an automation system to drive droplet movement on the DMF device, and (3) an absorbance reader to monitor the optical density (OD) of the cells, although this is a non-exhaustive list. The system in this example can be automated such that cell culture, OD monitoring and measurement, induction, and testing protein expression are all conducted on chip without manual intervention. This system also presents additional advantages for gene expression protocols as it minimizes chances for cross-contamination, presents greater control over experimental conditions, allows additional cultures to be induced simultaneously, and/or reduces significant costs for inducers (like IPTG) by minimizing the volumes required for induction. Although the system in this example is built for IPTG-based induction to facilitate OD monitoring, it can be used with other inducible systems or auto-inducible expression systems, e.g., for automating all fluidic operations to control conditions for protein expression without the need of an inducer. This example illustrates a proof-of-principle implementation of an automated workflow that was described to test a variety of induction conditions to determine the levels of protein expression of a red-fluorescent protein (RFP) gene. The utility and versatility of the AIMS were also demonstrated by testing the activity of key β-glucosidase (BGL) genes from Thermomicrobium roseum, Thermobaculum terrenum, and Rhodothermus marinus that may be useful in biomass hydrolysis for biofuel production, as an example embodiment, although other genes and cells may be used in other embodiments.

Materials and methods. All general-use reagents were purchased from Sigma, unless specified otherwise. E. coli DH5-alpha and BL21(DE3) strains and original pET16b vectors were generously donated from Dr. Vincent Martin. Strain and plasmids used for this study are shown in Table 4 (plasmids also made available from Addgene and ACS Synthetic Biology registry). Miniprep kits (cat no. BS413) and gel extraction kits (cat no. BS354) were purchased from BioBasic (Amherst, N.Y.). Beta-glucosidase substrate 4-methylumbelliferyl beta-D-glucopyranoside (MUG) was purchased from Carbosynth (cat no. EM05983, San Diego, Calif.).

TABLE 4

| Strains | Genotype | Source |
|---|---|---|
| E. coli DH5α | fhuA2 Δ(argF-lacZ)U169 | V. Martin |
| | phoA glnV44 Φ80 | |
| | Δ(lacZ)M15 gyrA96 recA1 | |
| | relA1 endA1 thi-1 hsdR17 | |
| E. coli BL21(DE3) | F- ompT gal dcm lon hsdSB(rB- | V. Martin |
| | mB-) λ(DE3 [lacI lacUV5-T7 | |
| | gene 1 ind1 sam7 nin5]) | |

| | Relevant | |
|---|---|---|
| Plasmids | characteristics | Source |
| pET16b | AmpR, pBR322 origin | V. Martin |
| pET_RFP | AmpR, mRFP | This study |
| pET_BGL1 | AmpR, BGL1 | This study |
| pET_BGL2 | AmpR, BGL2 | This study |
| pET_BGL3 | AmpR, BGL3 | This study |

Microfluidics device fabrication reagents and supplies included chromium coated with S1811 photoresist on glass slides from Telic (Valencia, Calif.), indium tin oxide (ITO)-coated glass slides, R_{S}=15-25 ohms (cat no. CG-61IN-S207, Delta Technologies, Loveland Colo.), FluoroPel PFC1601V from Cytonix LLC (Beltsville, Md.), MF-321 positive photoresist developer from Rohm and Haas (Marlborough, Mass.), CR-4 chromium etchant from OM Group (Cleveland, Ohio), and AZ-300T photoresist stripper from AZ Electronic Materials (Somerville, N.J.). Transparency masks for device fabrication were printed from CADArt (Bandon, Oreg.) and polylactic acid (PLA) material for 3D printing were purchased from 3Dshop (Mississauga, ON, Canada).

Device Design, Fabrication, and Assembly. Two digital microfluidic device geometries were used for this study which were made using Autocad. Design #1 used a linear array of electrodes with one reservoir electrode and design #2 used driving electrodes separated by gaps of 20 micrometers; electrode patterns and dimensions are listed in FIG. 5.

Device fabrication procedures are as follows. Briefly, chrome substrates were patterned using photolithography, developing, etching, and stripping methods. After patterning, these were coated with Parylene-C (˜5 micrometers) and FluoroPel 1601V (180 nm). Parylene was applied by evaporating 15 g of parylene C dimer in a vapor deposition instrument (Specialty Coating Systems, Indianapolis, Ind.) and the hydrophobic FluoroPel 1601V (Cytonix, Beltsville, Md., USA) was spin coated (1500 rpm, 30 s) and post-baked on a hot plate (180° C., 10 min). Unpatterned top plates were formed by spin-coating ITO with FluoroPel 1601V (as with bottom substrates).

Devices were assembled with the ITO top-plate and a patterned bottom plate separated by a spacer formed with one or four pieces of double-sided tape (70 or 280 micrometers respectively). Droplets were sandwiched between these two plates and were actuated by applying electric potentials between the two plates. Each electrode was connected to a contact pad (not shown in FIG. 5 for simplicity) that is interfaced with the pogo pin connector. Droplet motion was managed using the automated imaging feedback system. All reagents were manually loaded into the reservoirs using a pipettor.

Molecular cloning. The gene sequence for the Thermobaculum terrenum beta-glucosidase (BGL1) was obtained from NCBI (GenBank accession number WP_041425608.1) and was synthesized by Gen9 (now part of Ginko Bioworks) in a pGm9-2 backbone (sequence of BGL1). The gene was amplified by PCR with primers (shown below) introducing a 5′ XbaI and a 3′ BamHI restrictions sites.

TABLE 5

| | |
|---|---|
| Forward: | 5′-TGACTGACTCTAGAAATAATTTTGTTTAACTTT |
| | AAGAAGGAGATATACCATGGACCCGTATGAAGATCC |
| | GC-3′ |
| | (SEQ ID NO: 3) |
| Reverse: | 5′-GCATGCATGGATCCCTACAGGGTCAGACCATGA |
| | CCG-3′ |
| | (SEQ ID NO: 4) |

Individual PCR reactions used 10 microliters 5× Phusion buffer, 1 microliters dimethylsulfoxide (DMSO), 20 ng template DNA, individual dNTPs and primers to a final concentration of 200 micromolar and 0.5 micromolar, and distilled water up to 50 microliters. The following PCR thermocycling conditions were used: initial denaturation at 98° C. for 30 s followed by 35 cycles of denaturation at 98° C. for 10 s, annealing at 55° C. for 30 s and extension at 72° C. for 30 s/kb, and a final extension step at 72° C. for 10 min. PCR products were loaded into a 0.8% agarose gel in TAE buffer and resolved at 130 V for 30 min. The corresponding bands were extracted using a gel extraction kit. The gene was then digested using XbaI and BamHI restriction enzymes and ligated into a linearized pET16b vector backbone (see plasmid map in FIG. 6).

The ligation product was transformed into chemically competent E. coli DH5 alpha cells and plated on LB plates containing 100 micrograms/mL ampicillin (Amp). For transformation, 100 microliters of thawed competent cells were mixed on ice with 100 ng of the ligation product. This mixture was heat-shocked at 42° C. for 60 s after which cells were placed on ice for 1 min for recovery. 900 microliters of LB were added to the transformation mixture and the cells were incubated at 37° C. for 1 h. 200 microliters of this mixture were plated onto selective media. The following day, single colonies were inoculated in 5 mL of LB Amp media overnight and plasmids were extracted using a BioBasic miniprep kit. Finally, proper insertion of the gene was verified by digesting 2 micrograms of plasmid with XbaI and BamHI and running the product on a 0.8% agarose gel to look for the correct insert band size.

Protein expression. The plasmid containing the cloned BGL1 gene was first transformed into E. coli BL21(DE3) for recombinant expression. The transformed cells were inoculated overnight in a 5 mL pre-culture. The following day, the culture was diluted to OD 0.05 in a 100 mL starter culture and grown at 37° C. with 200 rpm shaking. Upon reaching OD 0.4, expression of the BGL1 gene was induced by addition of 1 mM IPTG and induction was carried out under the same growth conditions for 8 hours. The final induced culture was centrifuged at 4000 rpm for 5 min and the supernatant was discarded. The cell pellet was re-suspended in 2 mL lysis solution per 50 mL of initial culture. The lysis solution comprises 1 mg/mL lysozyme, 25 U/ml benzonase and 1 mM phenylmethanesulfonylfluoride (PMSF). Lysis was carried out for 30 min at room temperature and the lysates were diluted 100-fold in assay buffer containing 50 mM sodium citrate at pH 7 and stored at 4° C. before the assay.

BGL off-chip assay. In the assay, nine reactions used equal volumes of cell lysate and 4 mM of p-nitrophenyl-beta-D-glucopyranoside (pNPG) dissolved in the assay buffer. At 30 min intervals, 134 microliters from a reaction were added to 67 microliters of a 300 mM glycine-NaOH solution in a transparent flat bottom well plate to stop the reaction. Absorbance at 405 nm was immediately acquired after stopping each reaction on a TECAN infinite M200 plate reader with the following settings: 9 nm bandwidth, single reads per well, 25 flashes per reading, and 0 ms of settle time. Reactions with absorbance units >4 were diluted and the final absorbance was calculated from the diluted sample. The assay was repeated in triplicate and lysates from a transformed culture with an empty pET16b plasmid were used as a negative control.

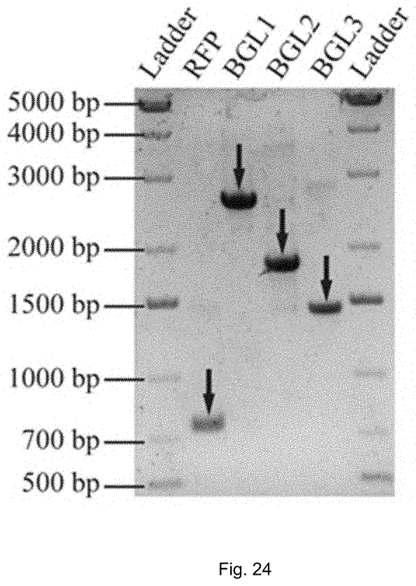

Plasmid preparation and transformation. The gene sequence for the reporter red fluorescence protein (RFP) was obtained from the iGEM registry (BBa_E1010) and the beta-glucosidase genes (BGL) from Thermomicrobium roseum (BGL1, GenBank accession number YP_002522957.1), Thermobaculum terrenum (BGL2, GenBank accession number WP_041425608.1), and Rhodothermus marinus DSM4252 (BGL3, GenBank accession number WP_012844561.1). BGL1 was synthesized by IDT (Coralville, Iowa) as a linear DNA fragment, and BGL2 and BGL3 were synthesized by Gen9 (now Ginko Bioworks). These genes were used for amplification by PCR (see Table 4 for primer sequences). Individual PCR reactions used 10 microliters 5× Phusion buffer, 1 microliter dimethylsulfoxide (DMSO), 20 ng template DNA, individual dNTPs and primers to a final concentration of 200 micromolar and 0.5 micromolar each, 0.5 microliters Phusion polymerase and distilled water up to 50 microliters. The following PCR thermocycling conditions were used: initial denaturation at 98° C. for 30 s followed by 35 cycles of denaturation at 98° C. for 10 s, annealing at 55° C. for 30 s and extension at 72° C. for 30 s/kb, and a final extension step at 72° C. for 10 min. PCR products were loaded into a 0.8% agarose gel in TAE buffer and resolved at 130 V for 30 min. The corresponding bands from a gel (FIG. 24) were extracted using a gel extraction kit. Primer sequences are shown in Table 6:

TABLE 6

| Gene | Orientation | Sequence |
|---|---|---|
| RFP | Forward | TGACTGACTCTAGAAATAATTTTGTTTAACTT |
| | | TAAGAAGGAGATATACCATGGCTTCCTCCGAA |
| | | GACGT |
| | | (SEQ ID NO: 5) |
| RFP | Reverse | GCATGCATGGATCCTTAAGCACCGGTGGAGTG |
| | | AC |
| | | (SEQ ID NO: 6) |
| BGL1 | Forward | TGACTGACTCTAGAAATAATTTTGTTTAACTT |
| | | TAAGAAGGAGATATACCATGAAACATCTGGTT |
| | | ACCACACTGC |
| | | (SEQ ID NO: 7) |
| BGL1 | Reverse | GCATGCATGGATCCCTACTGAACATCAATTTC |
| | | GGTCTGCA |
| | | (SEQ ID NO: 8) |
| BGL2 | Forward | TGACTGACTCTAGAAATAATTTTGTTTAACTT |
| | | TAAGAAGGAGATATACCATGGACCCGTATGAA |
| | | GATCCGC |
| | | (SEQ ID NO: 9) |
| BGL2 | Reverse | GCATGCATGGATCCCTACAGGGTCAGACCATG |
| | | ACCG |
| | | (SEQ ID NO: 10) |
| BGL3 | Forward | TGACTGACTCTAGAAATAATTTTGTTTAACTT |
| | | TAAGAAGGAGATATACCATGAGCGTAGCGCGG |
| | | TTT |
| | | (SEQ ID NO: 11) |
| BGL3 | Reverse | GCATGCATGGATCCCTAACCTTCCACCAAAGC |
| | | ATTTCTTG |
| | | (SEQ ID NO: 12) |

FIG. 24 illustrates gel electrophoresis of the PCR products derived from amplification of the pET16b vector containing the synthetic inserts RFP, BGL1, BGL2 and BGL3. Arrows show the bands with the expected weight for each PCR products, which were 678 bp (RFP), 2520 bp (BGL1), 1761 bp (BGL2), and 1359 bp (BGL3).

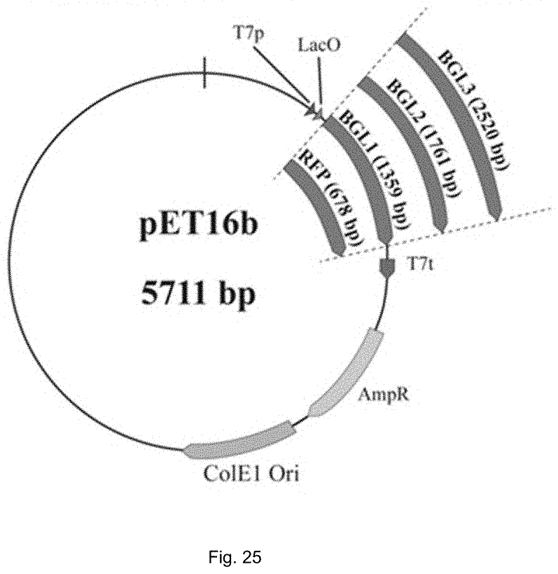

The recovered DNA was digested using XbaI and BamHI restriction enzymes (Thermo, Waltham, Mass.) for 4 hours at 37° C. and ligated into a pET16b expression vector that contains a T7 promoter and lacI coding sequence using T4 ligase (Thermo, Waltham, Mass.) for 1 hour at room temperature (see FIG. 25 for plasmid map). FIG. 25 shows a schematic of the plasmid used in the study: BGL and RFP were inserted downstream of a T7 promoter. For transformation, 100 microliters of thawed competent cells were mixed with 100 ng of the ligation product and placed on ice. This mixture was heat-shocked at 42° C. for 45 s after which cells were placed on ice for 1 min for recovery. 900 microliters of LB media were added to each transformation mixture and the cells were incubated at 37° C. for 1 h. 200 microliters of the final mixture were plated onto selective LB agar plates containing 100 micrograms/mL ampicillin and incubated at 37° C. overnight. Single colonies were picked the following day and inoculated into 5 mL of LB Amp overnight. Plasmids containing RFP and BGL genes were extracted from E. coli using a miniprep kit and were digested with XbaI and BamHI and verified on a gel to ensure proper insertion of the genes.

Conventional benchtop culture, induction, and expression. Chemically competent E. coli BL21(DE3) cells were transformed with the expression vector containing the cloned genes for induction. Cultures from single colonies were grown in 5 mL of LB media containing 100 micrograms/mL ampicillin (Amp) shaking at 200 rpm with constant 37° C. temperature overnight. These were diluted to a starter culture of OD 0.1 and grown under the same conditions until they reached an OD of 0.4. Optical densities at 600 nm were measured periodically in microcentrifuge tubes on a Varian Cary 50 Bio UV-vis spectrophotometer (Agilent Technologies, Santa Clara, Calif.). To initiate gene expression, the cultures were induced by adding 1 mM IPTG at OD 0.4 and were incubated under the same conditions for 4 h. Induced cultures were then collected in microcentrifuge tubes and stored at −20° C. for later use.

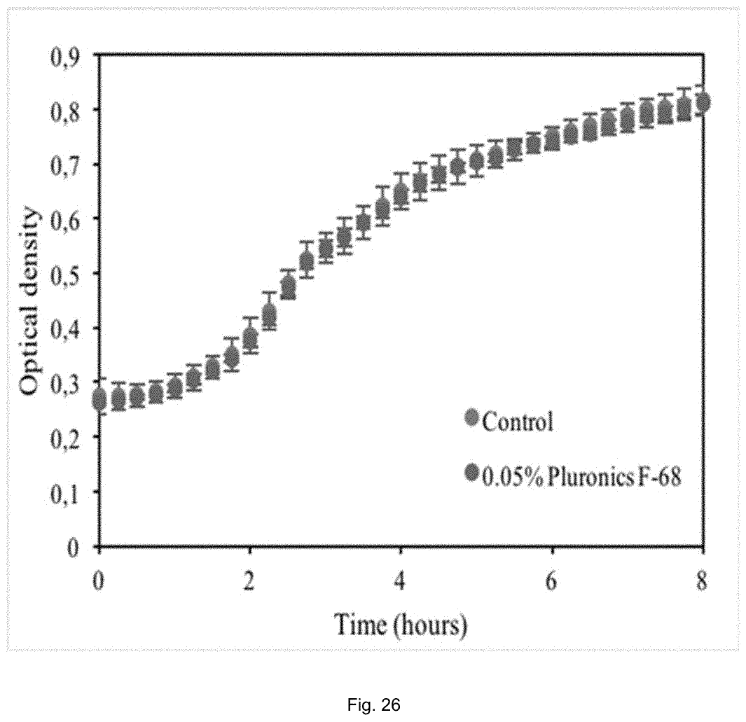

To obtain a macroscale growth curve, a 150 mL culture was started by diluting an overnight culture carrying an empty pET16b vector to OD 0.1 in selective media. The macroscale culture was incubated at 37° C. with 200 rpm shaking. The flask was taken out every 30 min to measure the optical density of triplicate 1 mL samples. OD was measured at 600 nm on the Varian Cary 50 spectrophotometer. The experiment was carried out until OD reached a plateau, and a growth curve for the macro-scale culture was plotted. Since cells in Pluronics F-68 are being cultured on microfluidics, the effects of Pluronics F-68 are also tested on bacteria growth and no detrimental effects on their growth are discovered (FIG. 26). FIG. 26 shows a growth curve for BL21 E. coli cultured under normal culturing conditions with and without 0.05% Pluronics F-68.

For inducer concentration optimization carried out in the macroscale, starter cultures with a RFP plasmid were prepared at OD 0.1 from overnight inoculations. The cultures were grown at 37° C. with shaking and were induced upon reaching OD 0.4. 45 mL of the culture was induced at 200 micromolar and diluted with fresh media to generate the following IPTG concentrations: 200, 133.3, 88.9, 59.3, 40, 26.7, 17.8, and 11.9 micromolar. These sub-cultures were prepared in triplicates along with a non-induced control and were induced at 37° C. and shaken for 4 hours. After induction, 200 microliters of each culture were loaded onto a 96-well plate and fluorescence at 612 nm was measured with 582 nm excitation on a TECAN Infinite M200 plate reader (Mannedorf, Switzerland) with the settings: gain of 75, 25 flashes and 20 microseconds integration time. The fluorescence intensity with increasing IPTG concentration was plotted on a logarithmic scale to generate a dose-response curve.

Microfluidic Device Fabrication. Devices were designed using AutoCAD 2016 (Autodesk, San Rafael, Calif.) and fabricated in the Concordia Silicon Microfabrication Lab (ConSIM). The fabrication procedure followed a previous protocol using high resolution 25,400 dpi transparency masks printed by CAD/Art services. Briefly, glass substrates pre-coated with S1811 photoresist (Telic, Valencia, Calif.) were exposed to UV for 8 s on a Quintel Q-4000 mask aligner (Neutronix Quintel, Morgan Hill, Calif.) to imprint the transparency masks design. These were developed in MF-321 for 2 min with shaking and rinsing with DI water. Developed slides were then baked at 115° C. for 1 min before etching in CR-4 chromium etchant until the pattern was clearly visible. The remaining photoresist was then removed in AZ-300T stripper for 2 min.

After rinsing with DI water and drying, a silane solution comprising deionized water, 2-propanol and (trimethoxysilyl)-propyl methacrylate (50:50:1) was added to the devices in a pyrex dish for 15 min. Devices were primed for dielectric coating with Parylene-C (7.2 micrometer) in a SCS Labcoter 2 PDS 2010 (Specialty Coating Systems, Indianapolis, Ind.), and coated with Fluoropel PFC1601V (Cytonix, Beltsville, Md.) in a Laurell spin coater (North Wales, Pa.) set to 1500 rpm for 30 s with 500 rpm/s acceleration followed by 10 min baking at 180° C.

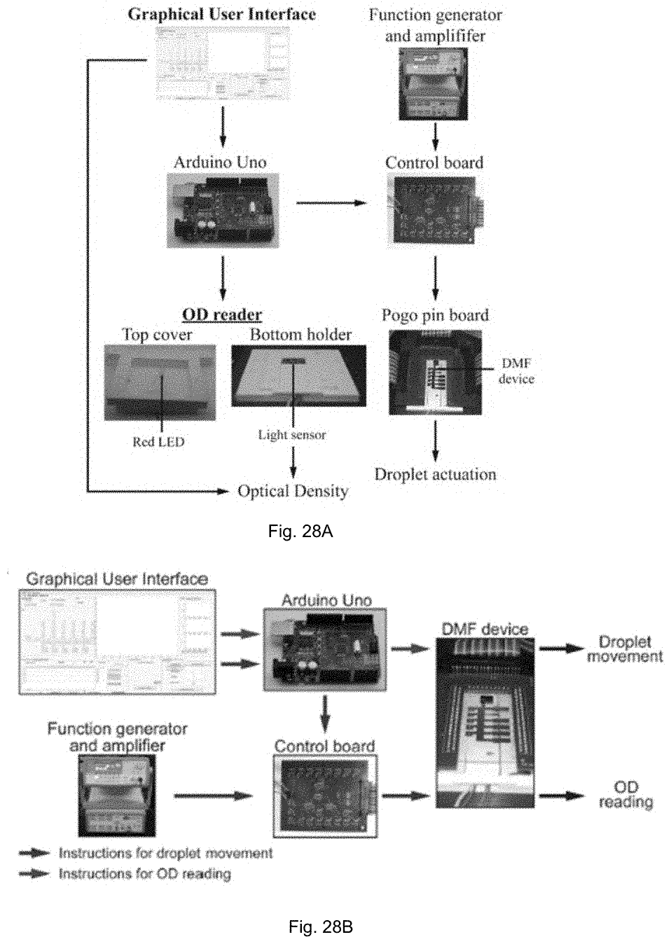

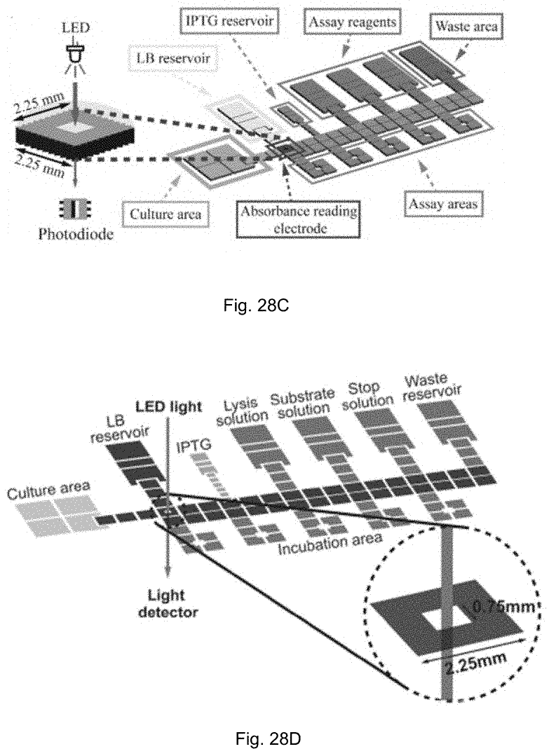

Automated Induction Microfluidics System (AIMS). Referring to FIGS. 28A and 28B, there are shown embodiments of an automated induction microfluidics system (AIMS). Referring to FIG. 28A, the schematic illustrates the relationships between the function generator and amplifier, the control board bearing the solid state switches for high voltage, the Arduino Uno, the pogo pin board and the optical density (OD) reader with DMF device. Low voltage signals (5V DC) are delivered to the Arduino to activate the switches on the control board to deliver high voltage (˜100 VRMS) to the DMF device via pogo pins. To automate cell culture, induction, and analysis of protein expression, user programs a droplet movement sequence by clicking on the graphical user interface to initiate droplet movement.

Referring to FIG. 28A, there is shown schematic of the device. A cell culture area bearing four square electrodes (4.5×4.5 mm each) are used to semi-continuously mix the mother culture droplet. To monitor OD, the mother droplet is extended to the absorbance-reading electrode (left, expanded view). If the OD reading surpasses the threshold, a droplet of IPTG is dispensed and mixed with a daughter droplet. Next, this will start one of two programs: concentration or time-course, which will initiate droplet movement sequences and start incubation in the assay regions.

FIG. 28B also illustrates the relationships between a function generator and amplifier, a control board, Arduino Uno, a pogo pin board and an OD reader with DMF device.

As depicted in FIG. 28A, the AIMS was comprised of a 3D printed top cover with a 600 nm LED (Digikey, Cat no. 1497-1021-ND, Winnipeg, MB) and a bottom holder (see SI for top and bottom holder fabrication) containing a luminosity sensor (TSL2561, Adafruit, New York, N.Y.). To measure optical density, devices are placed in a slot in the bottom holder that is approximately 8 mm below the LED and 4 mm above the lux sensor. Alignment marks were designed on the device and on the bottom holder to align the absorbance window on the device with the lux sensor to minimize fluctuations in the lux measurements. The lux sensor was programmed and managed using an Arduino Uno controller connected to the graphical user interface to display the measured luminosity values.

FIG. 28C illustrates a schematic of a DMF device. FIG. 28D illustrates a schematic of a DMF device. Table 7 illustrates examples of electronic components for manufacturing a control system, according to this example.

TABLE 7

| | | | | Qty/ | Symbols on | Manufacturer Part | | |
|---|---|---|---|---|---|---|---|---|
| Symbols | Item | Description | Ref Des | board | the board | Number | Manufacturer | BOARDS |
| R | 1 | 4.7k Ohm +/− | R1, R2, R47, | 4 | R1, R2, R47, R48 | RC0402JR-074K7L | Yageo | CONTROL |
| | | 5% 0.063 W, | R48 | | | | | BOARD |
| | | 1/16 W Chip | | | | | | |
| | | Resistor 0402 | | | | | | |
| | | (1005 Metric) | | | | | | |
| | | Moisture | | | | | | |
| | | Resistant | | | | | | |
| | | Thick Film | | | | | | |
| R | 2 | 39k Ohm +/− | R3, R49 | 2 | R3, R49 | RC0402FR-0739KL | Yageo | CONTROL |
| | | 1% 0.063 W, | | | | | | BOARD |
| | | 1/16 W Chip | | | | | | |
| | | Resistor 0402 | | | | | | |
| | | (1005 Metric) | | | | | | |
| | | Moisture | | | | | | |
| | | Resistant | | | | | | |
| | | Thick Film | | | | | | |
| R | 3 | 220 Ohm +/− | R17, R18, | 40 | [R17-R36], | RC0402JR-07220RL | Yageo | CONTROL |
| | | 5% 0.063 W, | R19, R20, | | [R63-R82] | | | BOARD |
| | | 1/16 W Chip | R21, R22, | | | | | |
| | | Resistor 0402 | R23, R24, | | | | | |
| | | (1005 Metric) | R25, R26, | | | | | |
| | | Moisture | R27, R28, | | | | | |
| | | Resistant | R29, R30, | | | | | |
| | | Thick Film | R31, | | | | | |
| | | | R32, R33, | | | | | |
| | | | R34, R35, | | | | | |
| | | | R36, R63, | | | | | |
| | | | R64, R65, | | | | | |
| | | | R66, R67, | | | | | |
| | | | R68, R69, | | | | | |
| | | | R70, R71, | | | | | |
| | | | R72, | | | | | |
| | | | R73, R74, | | | | | |
| | | | R75, R76, | | | | | |
| | | | R77, R78, | | | | | |
| | | | R79, R80, | | | | | |
| | | | R81, R82 | | | | | |
| R | 4 | 1k Ohm +/− | R4, R5, R6, | 40 | [R4-R16], | RT0402DRD071KL | Yageo | CONTROL |
| | | 0.5% 0.063 W, | R7, R8, R9, | | [R37-R46], | | | BOARD |
| | | 1/16 W Chip | R10, R11, | | [R50-R62], | | | |
| | | Resistor 0402 | R12, R13, | | [R83-R92] | | | |
| | | (1005 Metric) | R37, R38, | | | | | |
| | | Moisture | R39, R40, | | | | | |
| | | Resistant | R41, R42, | | | | | |
| | | Thick Film | R43, R44, | | | | | |
| | | | R45, R46, | | | | | |
| | | | R50, R51, | | | | | |
| | | | R52, R53, | | | | | |
| | | | R54, R55, | | | | | |
| | | | R56, R57, | | | | | |
| | | | R58, R59, | | | | | |
| | | | R83, | | | | | |
| | | | R84, R85, | | | | | |
| | | | R86, R87, | | | | | |
| | | | R88, R89, | | | | | |
| | | | R90, R91, | | | | | |
| | | | R92 | | | | | |
| C | 5 | CAP CER | C1, C2, C3, | 8 | [C1-C8] | CC0402KRX7R7BB104 | Yageo | CONTROL |
| | | 0.1UF 16V | C4, C5, C6, | | | | | BOARD |
| | | X7R 0402 | C7, C8 | | | | | |
| M | 6 | I/O Expander | M1, MX2 | 2 | MAX7300 | MAX7300AAI+ | Maxim | CONTROL |
| | | 28 I2C | | | | | Integrated | BOARD |
| | | 400 kHz 28- | | | | | | |
| | | SSOP | | | | | | |
| I | 7 | Buffer, | I1, I2, I3, I4, | 6 | [I1-I6] | 74AC540SC | Fairchild/ON | CONTROL |
| | | Inverting | I5, I6 | | | | Semiconductor | BOARD |
| | | Element 8 Bit | | | | | | |
| | | per Element | | | | | | |
| | | Push-Pull | | | | | | |
| | | Output 20- | | | | | | |
| | | SOIC | | | | | | |
| U | 8 | RELAY | U1, U2, U3, | 40 | [U1-U40] | AQW216EHA | Panasonic | CONTROL |
| | | OPTO 600V | U4, U5, U6, | | | | Electric Works | BOARD |
| | | 0.04A 8SMD | U7, U8, U9, | | | | | |
| | | | U10, U11, | | | | | |
| | | | U12, U13, | | | | | |
| | | | U14, U15, | | | | | |
| | | | U16, U17, | | | | | |
| | | | U18, U19, | | | | | |
| | | | U20, U21, | | | | | |
| | | | U22, U23, | | | | | |
| | | | U24, U25, | | | | | |
| | | | U26, U27, | | | | | |
| | | | U28, U29, | | | | | |
| | | | U30, U31, | | | | | |
| | | | U32, | | | | | |
| | | | U33, U34, | | | | | |
| | | | U35, U36, | | | | | |
| | | | U37, U38, | | | | | |
| | | | U39, U40 | | | | | |
| AD | 9 | CONN | AD0, AD0., | 3 | AD0, AD0., | 15912080 | Molex, LLC | CONTROL |
| | | HEADER | AD1, AD1. | | AD1, AD1. | | | BOARD |
| | | 8POS .100″ | | | | | | |
| | | VERT TIN | | | | | | |
| Header | 10 | STACKING | HEADER | 1 | Header | 85 | Adafruit | CONTROL |
| | | HEADER | | | | | Industries | BOARD |
| | | ARDUINO | | | | | LLC | |
| | | SHIELD | | | | | | |
| H1 | 11 | CONN | H1 | 1 | H1 | 5499913-9 | TE | CONTROL |
| | | HEADER | | | | | Connectivity | BOARD |
| | | RT/A 40POS | | | | | AMP | |
| | | .100 GOLD | | | | | Connectors | |
| CONX | 1 | CONN | CONX1, | 3 | CONX1, | 5499913-9 | TE | POGO PIN |
| | | HEADER | CONX2, | | CONX2, | | Connectivity | BOARD |
| | | RT/A 40POS | CONX3 | | CONX3 | | AMP | |
| | | .100 GOLD | | | | | Connectors | |
| N/A | 2 | CONN PIN | U$1 | 104 | | 0906-1-15-20-75-14-11-0 | Mill-Max | POGO PIN |
| | | SPRING- | | | | | Manufacturing | BOARD |
| | | LOADED | | | | | Corp. | |
| | | PCB GOLD | | | | | | |

Droplet motion on the devices was managed using an automated control system. It used a custom MATLAB (Natlick, Mass.) program interfaced to an Arduino UNO that controls the states of a network of high-voltage relays (AQW216 Panasonic, Digikey, Winnipeg, MB). The control board is connected to a function generator (33201A Agilent, Allied Electronics, Ottawa, ON) and a high-voltage amplifier (PZD-700A, Trek Inc., Lockport, N.Y.) that delivers 130-270 VRMS sinusoidal signals to the mated pogo-pin board. Specifically, the inputs of the relays are connected to the function generator/amplifier combination and the outputs are mated to the pogo pin board. Controlling the logic of the individual switches is done through an I²C communication protocol using an I/O expander (Maxim 7300, Digikey, Winnipeg, MB). In practice, the user inserts the device into the OD reader, loads the reagents onto the device, and then inputs a series of desired droplet movement steps such that induction (and cell culture and analysis) will be performed automatically by the AIMS. An example list of components that can be used to manufacture a microfluidics control system is included in Table 7.

Microfluidic automated culture, induction, expression. The above protocols for the conventional benchtop experiments were adapted to the volumes used on the microfluidic device and supplemented with 0.05% Pluronics F-68. Pluronics additives were used to prevent any proteins or cell adsorption on the DMF device. Prior to the experiment, the device (FIG. 28B) was inserted between the OD reader of the AIMS setup and the pogo pin interface. A droplet containing media with cells was loaded onto the mother culture area and the bottom plate was mated with an ITO top plate for grounding to complete the device configuration. During the experiment, this setup was placed in an incubator to maintain the system temperature at 37° C., with an open water container to provide humidity and to prevent droplet evaporation on the device.

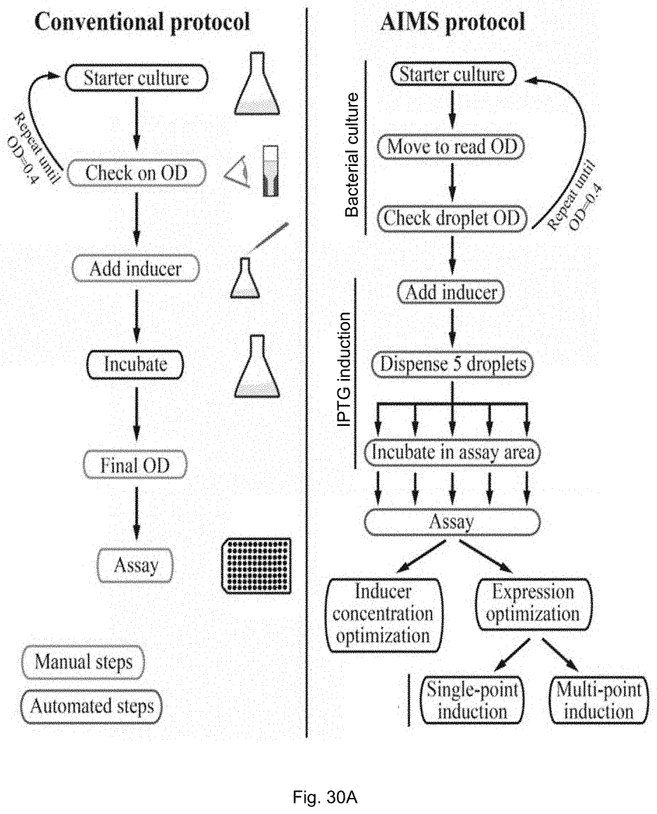

To generate a growth curve, the mother culture was initialized by diluting an overnight culture with fresh media containing 0.05% Pluronic F-68 to a low OD (˜0.1). 14 microliters of this culture were loaded onto the culturing area of the DMF device and was semi-continuously mixed at a frequency of one actuation every 45 s (with 700 ms of actuation time) to ensure uniform cell density in the mother culture (see FIG. 30A, mixing).

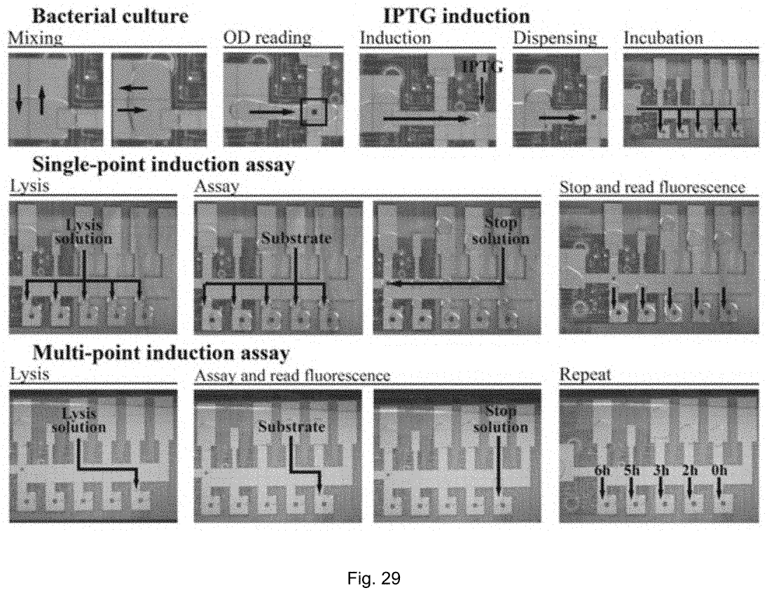

Referring to FIG. 29, there is shown a sequence of droplet operation using AIMS according to one example. In “Bacterial culture,” the mother drop was mixed by the AIMS interchanging vertical and horizontal directions. The mother drop was extended and actuated to the absorbance window to measure the OD of the culture. In “IPTG induction,” a droplet of IPTG is dispensed and mixed with the mother culture droplet. Five daughter droplets are then dispensed and incubated in the five assay areas. In “Single-point induction assay,” the BGL assay used successive mixing of the induced culture with a lysis solution, incubation with the MUG substrate, followed by the addition of a stop solution.

FIGS. 30A and 30B show comparisons of the conventional and microfluidic induction protocol. The conventional protocol uses large volumes (˜mL) to start the cell culture and frequently requires manual monitoring of the OD. Once the culture reaches the threshold OD, the user pipettes an aliquot of an inducer (e.g., IPTG) into the culture and continues culturing until ready for a biological assay. Typically, the user requires another liquid handling platform for the biological assay (e.g., well-plate). The AIMS protocol only requires initial pipetting steps (reagents, cells in media, inducer) while all other induction and assay steps are automated. The “Inducer concentration” program was used to optimize IPTG concentrations, and the “Expression optimization” program was used to screen different enzymes (Single-point induction) and expression conditions of the highest active enzyme (Multipoint induction).

Illuminance measurements (lux) were carried out from the absorbance window on the device using the luminosity sensor. A blank (i.e., a droplet of LB media and no cells) value was acquired before every sample reading to calculate the OD, using the equation:

$$A = log ( \frac{{I}_{0}}{I} ) / 0.028 ,$$

where A is the measured absorbance in OD, I₀ is the blank light intensity value, and I is the light intensity reading from the sample. The OD value is divided by 0.028 to correct for the path length of readings across the 280 micrometer of height gap.

During a period of cell growth, induction is used to initiate protein expression. The induction procedure starts with actuating the mother droplet containing the bacteria to the absorbance window to measure the OD (see FIG. 30A, OD reading). If the calculated OD is below the threshold OD of 0.4, the mother culture would go back to the mixing area and continue mixing for 10 min until the next OD reading. However, if the OD reaches the threshold, the control system would trigger an induction program to start by dispensing a droplet of IPTG to mix with the culture. This will initiate one of two programs: inducer concentration or expression optimization program.

In the inducer concentration optimization program, three unit droplets of 1.42 microliters containing transformed RFP-cells were dispensed from the culture region and mixed with 0.3 microliters of 3.24 mM IPTG. This droplet was actuated to an empty reservoir and one daughter droplet was split from this reservoir and actuated to the incubation region. Another unit droplet from the mother culture was then mixed into the reservoir and split again to generate 2:1 serial dilutions of IPTG. After each split, the droplets were actuated to their respective assay spot. To assess the impact of IPTG concentration on gene expression, the RFP expression was evaluated after four hours by placing the device on top of a well-plate cover and then inserted into in a CLARIOStar plate reader (BGM labtech, Ortenberg, Germany) to measure fluorescence emission at 612 nm with excitation at 582 nm using a well scanning program with scan matrix=30×30, scan width=6 mm, a focal height=7.2 mm, and with a gain=2905.

In the expression optimization program, two assays (single-point and multi-point) were conducted to show the utility of the system and to identify highly active BGL enzymes. In single-point induction, a 2 microliter droplet of 11 mM IPTG was mixed with a single culture droplet, which was then returned to the culture mixing area to mix and induce the entire culture (FIG. 30A, Induction). Five induced daughter droplets were dispensed and actuated to their respective incubation spots (FIG. 29, Incubation). After four hours of incubation, each of the droplets on the spot was mixed with a 1.42 microliter 1× lysis solution droplet to break open the cells to analyze the BGL enzymes (FIG. 30A, Lysis). After 10 min of lysis at room temperature, a 1.42 microliter droplet containing 150 mM sodium-citrate and 6 mM MUG was added to each assay area and were incubated for different durations (0, 15, 30, 45 and 60 min). The reaction was stopped by the addition of a 1.42 microliter droplet of 0.4 M glycine-NaOH (FIG. 30A, Stop and Read Fluorescence). To assess the BGL activity, the device was placed on a well-plate cover and into a well-plate reader to measure the fluorescence intensity at 449 nm upon 368 nm excitation, with the same settings as in the inducer concentration program except for a focal height of 4.0 mm and gain of 664. The fluorescence intensity of each droplet was taken for analysis.

In the multi-point induction assay, a culture of low OD (˜0.1) was grown and induced with the same volume and concentration as in the single-point program. Upon induction, five sub-cultures were lysed and assayed after 0, 2, 3, 5, and 6 h of incubation (FIG. 30A, Multi-point induction assay). Lysis was carried out for 10 min and each droplet was incubated with MUG for 30 min before quenching and fluorescence reading. The same settings were used for fluorescence measurement as in the single-point induction assay.

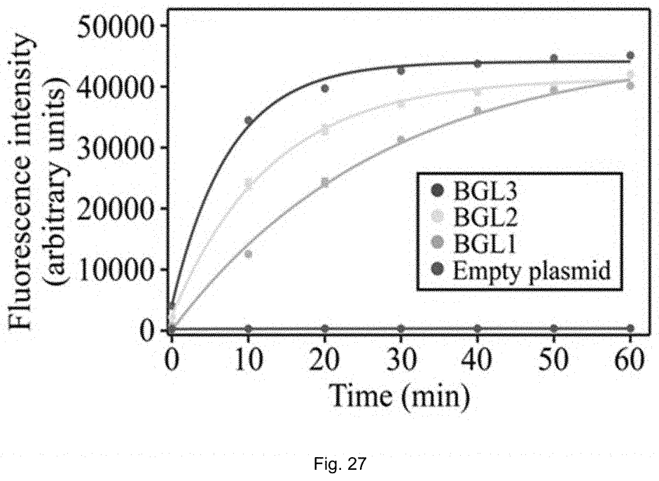

Referring to FIG. 27, there is illustrated expression optimization assay to discover highly active BGL conducted in well-plates. The activity of three different BGLs in the presence of 2 mM MUG were measured by fluorescence intensity (λₑₓ, lambda(ex)=369 nm and λₑₘ, lambda(em)=449 nm) over 60 min.

Characterization of the AIMS. A wide range of synthetic biology applications such as strain optimization require the use of induction. One example is to study biological parts or tools affecting recombinant protein expression in E. coli or yeast to improve protein yields or understand patterns of gene expression. Typically, induction follows a manual procedure with constant monitoring of cell density and manual addition of the inducer at a specific time point. In this example, the AIMS used digital microfluidics and is capable of culture, induction, and protein analysis without manual steps (FIG. 30).

FIG. 30B shows a comparison of the conventional and microfluidic induction protocol. The conventional protocol uses large volumes (˜mL) to start the cell culture and frequently requires manual monitoring of the OD. Once the culture reaches the threshold OD, the user pipettes an aliquot of an inducer (e.g., IPTG) into the culture and continues culturing until ready for a biological assay. Typically, the user requires another liquid handling platform for the biological assay (e.g., well-plate). The AIMS protocol only requires initial pipetting steps (reagents, cells in media, inducer) while all other induction and assay steps are automated. The “Inducer concentration” program was used to optimize IPTG concentrations, and the “Expression optimization” program was used to screen different enzymes (Single-point induction) and expression conditions of the highest active enzyme (Multi-point induction). The numbers in the AIMS protocol refer to the steps described in FIG. 29.

The primary function of the AIMS was to automate induction, which requires initial cell culturing in this example. As shown in FIG. 28C, the device was designed such that cell culture takes place in a 20 microliter droplet containing media and cells (with a starting OD of 0.1), which is termed “mother culture.” In initial experiments, the mother culture was continuously mixed to ensure uniform distribution of gases and nutrients and especially the cells themselves. However, it was observed biofouling after two hours of culturing which was not enough to reach the OD for induction. It has been reported elsewhere with droplet contents being mixed at rates up to 10-50× faster than diffusion alone using an array-based format of electrode. As shown in FIG. 29, the mixing step comprised of sequence of four movements that moved the mother culture in a horizontal and vertical directions. There are possibilities of moving the droplet in a more complex rearrangement (e.g., a figure-eight pattern) or resonating the droplet. However, these can require either more actuations or allowing the droplet to rest, which may lead to faster biofouling on the device. Faster actuation times <700 ms were initially tried in this device, but the droplet would not move to the activated electrode or slower actuation times but the droplet would biofoul the surface preventing further droplet movement. A balance was struck at 0.7 s (and every 45 s mixing frequency) when droplets would move while preventing any biofouling. Furthermore, the simple horizontal and vertical movement was adequate for induction and analysis since it provided a homogeneous distribution of the cells in the droplet.

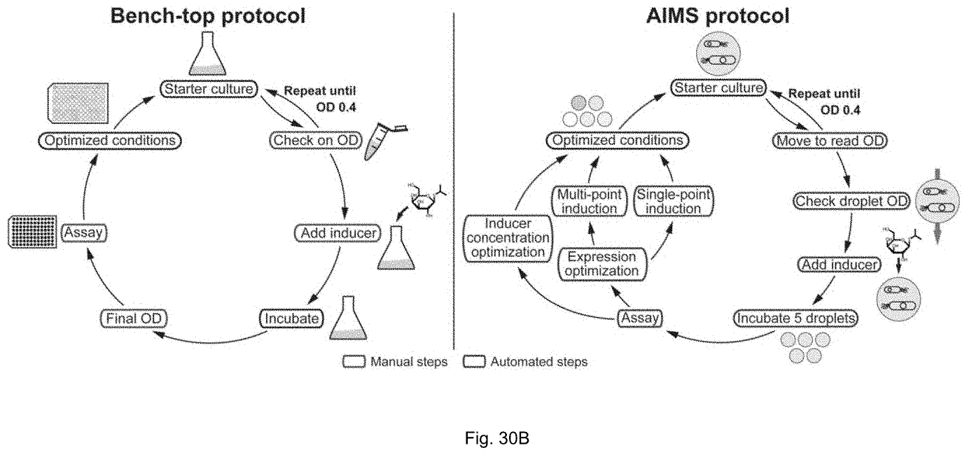

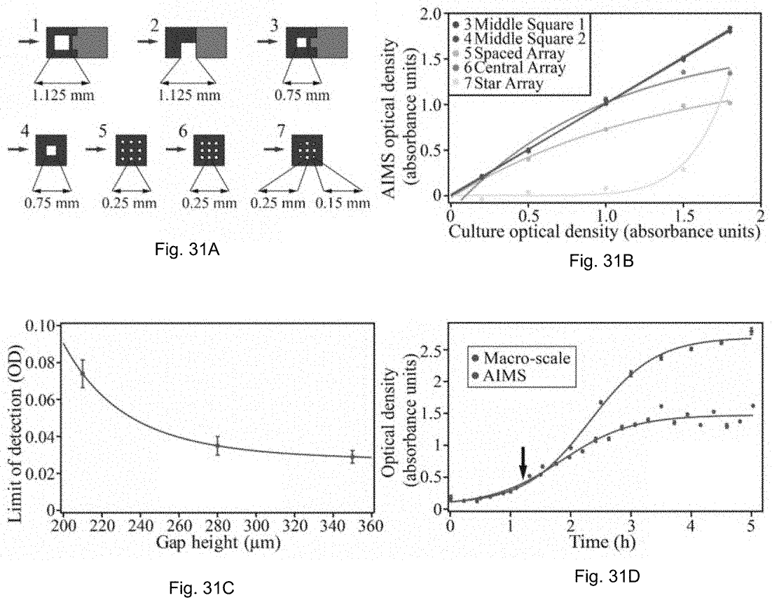

FIGS. 31A, 31B, 31C and 31D illustrate characterization of the AIMS used in this example. In FIG. 31A, a schematic of the different absorbance windows tested in this study is shown. In FIG. 31B, there is shown a calibration curve of bacterial cultures of different OD were measured in a spectrophotometer. The same samples were verified with the AIMS system. In FIG. 31C, there is shown a curve showing the limit of detection for a given inter-spacer height (between top and bottom plate). The limit of detection was calculated by measuring the OD using the AIMS of a blank sample (i.e., media with no cells) and adding three times the standard deviation. In FIG. 31D, there is shown representative growth curves of bacteria on the benchtop or using the AIMS. Benchtop measurements were conducted using a well-plate reader and grown in flasks while microscale measurements were conducted on the AIMS. The arrow indicates the point of induction (OD=0.4). For FIGS. 31B-31D, error bars represent +/−1 standard deviation across triplicates.

Next, to facilitate absorbance measurements, a variety of different shaped electrodes for cell density analysis. As shown in FIG. 31A, seven different transparent windows for measuring OD were tested. There are two criteria that were used in this example to determine the optimal electrode: 1) droplets move reliably onto the electrode, and 2) the range of OD measurements that can be accurately measured (i.e., resolution). To test droplet movement, a droplet from the mother culture was dispensed and actuated to the transparent electrode. Most of the evaluated electrodes (2-7) did not hinder droplet movement as the droplets reliably moved over the window. However, for electrode 1 (with a window of 1.125 mm), droplets were either sluggish in their movement or did not move over the window. This electrode was designed with a transparent region that is ½ of the area of the square electrode, which is not favorable since electrodynamic forces that are required to move the droplet are weaker when the electrode area is reduced. Overall, the most reliable movement on top of the absorbance electrode was observed by extending the mother culture onto the electrode rather than dispensing (FIG. 30A, OD reading).

Next, the range of OD measurements that can be observed with windows 2-7 was tested. Dilutions of bacterial cultures were created with different ODs (confirmed by the Varian Cary 50 Bio UV-vis spectrophotometer) and measured their OD with the AIMS. As shown in FIG. 31B, the results of the verification for multiple OD samples were plotted. The star-array window (electrode 7) did not give the expected linear range of values, which was also observed with the central (electrode 6) and the spaced (electrode 5) array. This is most likely due to the central transparent window being too small for reproducible measurements. However, using a middle square electrode (electrodes 3 and 4) showed favorable results in terms of linearity, resolution, and accuracy. Table 8 below shows the summary of the results and while the strategy of using a middle electrode worked well in the current design. Other possibilities for the device include integrating optical fibers or waveguides to increase the sensitivity of the measurements. In this particular device, Number 4 was used as the chosen absorbance window used for the AIMS.

TABLE 8

| | Description of | Droplet | | |
|---|---|---|---|---|
| Number | transparent window | movement? | Slope | r² |
| 1 | Large middle square | NO | NA | NA |
| 2 | Corner square | YES | NA | NA |
| 3 | Small middle square 1 | YES | 1.016 | 0.999 |
| 4 | Small middle square 2 | YES | 0.998 | 0.999 |
| 5 | Spaced array | YES | 1.1* | 0.997* |
| 6 | Central array | YES | 0.7* | 0.999* |
| 7 | Star array | YES | NA | NA |

*These values were obtained from the linear portion of the standard curves.

An advantage of using digital microfluidics for automated induction is that the vertical path length for absorbance measurements can be easily adjusted. Ideally, the larger the path length, the more sensitive the measurements will be at low absorbance (due to Beer-Lambert law). Here, three different gap heights were tested and the limit of detection of the OD measurements using AIMS was measured. Initially, small spacer thicknesses <140 micrometer between top and bottom plates in the devices were tried since it is the range of gap heights typically used for biological assays on DMF devices. However, at these lower gap heights, sensitive and reproducible OD measurements may not be achieved. This led to the use of larger heights (210, 280, and 350 micrometers) to determine the limit of detection by measuring the OD for droplets containing only media. A gap height of 350 micrometers gave the lowest limit of detection, 0.029 OD units (FIG. 31C). However, a commonly observed problem at these gap heights on the devices is the reliability of dispensing. In fact, droplet dispensing for the media with cells and the repetitive dispensing from a reservoir were nearly impossible. Increasing the voltage to improve droplet movement and dispensing as suggested by others were also tried, but this frequently led to electrolysis or dielectric breakdown on the device. Therefore, a 280 micrometer spacer was used in this example since it gave an appropriate limit of detection and was reproducible in terms of droplet dispensing and movement.

To ensure induction at proper times, the growth rates of bacteria on the AIMS was compared to those cultured by conventional means. As described in the methods section, the culture conditions of both systems were similar. As shown in FIG. 31D, the growth of bacteria had a similar trend in the exponential region of the curve but showed significant differences in doubling times with 36.80+/−0.36 and 72.88+/−2.30 min for conventional and AIMS cultures respectively (two-tailed paired t-test; P-value=0.018). Differences in the stationary phase were observed and it is believed that the variations in this phase between the micro- and macro-scale systems may be caused by a number of factors. The most likely factor is the mixing efficiency since there is semi-continuously mixing on the microfluidic device while continuously mixing in the macroscale. Differences in mixing can result in differences in dissolved gases and nutrients in the culture, which can make the bacteria cells enter the stationary phase faster than expected. In addition, the shorter path lengths in the microscale compared to the macroscale (280 micrometers vs. 1 cm) can also give rise to variances in the OD measurements. Although differences in the stationary phase were observed, induction occurs in the early exponential phase (˜0.3-0.4 OD) which is similar in both platforms.

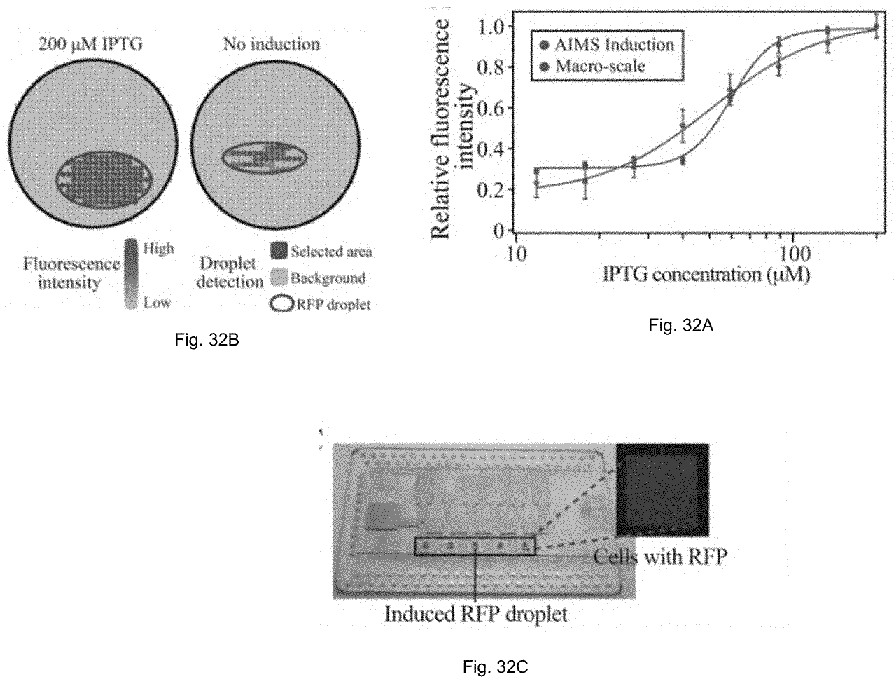

Inducer concentration optimization and monitoring gene expression. Referring to FIG. 32A, there is shown a comparison of the dose-response curves of IPTG using the AIMS and in macro-scale cultures. Error bars represent +/−1 standard deviation across triplicates. Referring to FIG. 32B, there is shown a RFP signal detected by fluorescent scan over an induced and non-induced droplet of culture. Fluorescence was measured with an excitation wavelength of 582 nm and an emission wavelength of 612 nm (refer to methods for specific well-plate settings). Referring to FIG. 32C, there is shown a picture showing five regions on the device that contain droplets were induced with IPTG. An expanded inset shows a droplet in the assay area with cells expressing RFP.

A key advantage of the AIMS is the potential of analyzing protein expression after induction directly on the same device. To illustrate this point with the AIMS, the system was tested with an IPTG inducible expression vector carrying a red fluorescent protein (RFP) gene downstream of a T7 promoter. Bacteria cells were cultured until OD 0.4 and induced using different IPTG concentrations (generated on-chip) to evaluate the optimal concentration for induction (FIG. 32A). As shown, the dose-response curve in both macro-scale and microfluidics devices followed a sigmoidal profile (e.g., a Hill function) with highest protein production after four hours at IPTG concentrations above 200 micromolar. At lower concentrations of IPTG (typically <30 micromolar), protein production was constant (comparable to basal levels), which is expected at these concentrations. Some differences in the shapes of the curves were observed, specifically in their steepness. This is not a surprise given the significant differences between both systems (in terms of volume, E-field actuation, optical detectors, mixing efficiency of samples, etc.) However, this can be improved by integrating “sensitivity tuners” or adding multiple protein-binding domains or transcriptional cascade systems into the cell that will adjust the effective binding cooperativity and improve cooperative binding of multiple transcription factors to the same promoter for transcriptionally regulated gene expression. Despite these differences, the system is capable of automating induction and monitoring gene expression, which can be extended to other types of induction assays (see expression optimization section).

Since fluorescence is used as a read-out for the protein production, an optical plate reader was used for analysis since the devices can be easily integrated with offline detectors. Using these optical detectors, only the droplet area can be detected and therefore there is no risk of other fluorescent signals interfering with the desired signals. In addition, this readout is the last step of the process and therefore only required the transfer of the device into the plate reader, and no additional pipetting steps or fluid handling steps are needed. As shown in FIG. 32B, the droplet can be selected by the well-plate software and can clearly distinguish between the droplet and its surrounding area and the difference between a low-fluorescence (no IPTG) and a highly fluorescent droplet (200 micromolar IPTG). This shows that this device is compatible with external detectors and can be used as an alternative for end-point fluorescence detection. In the future, it is proposed to integrate in-line fluorescent detectors or variations of other types of assays which require induction and use absorbance of fluorescence as a readout, e.g., genetic element screening and/or tuning gene expression.

As depicted in FIG. 32C, the method was carried out in a 5-plex format, but even higher levels of multiplexing of AIMS can be used in other embodiments, particularly with “hybrid” microfluidic techniques, which can increase throughput and analysis of 1000s of samples. In addition, the experiments reported here allow 10,000-fold reduction in bacterial culture volumes compared to bench-scale methods (15 microliters in microscale vs. 150 mL in batch scale) and at least 40-fold reduction in assay volumes (5 microliters on the device compared to 200 microliters in a 96-well plate). This system also allows an automated induction and gene expression analysis without intervention. The new system described here in this example may be particularly useful for applications involving precious and costly reagents and for induction assays that require multiple dilutions or conditions.

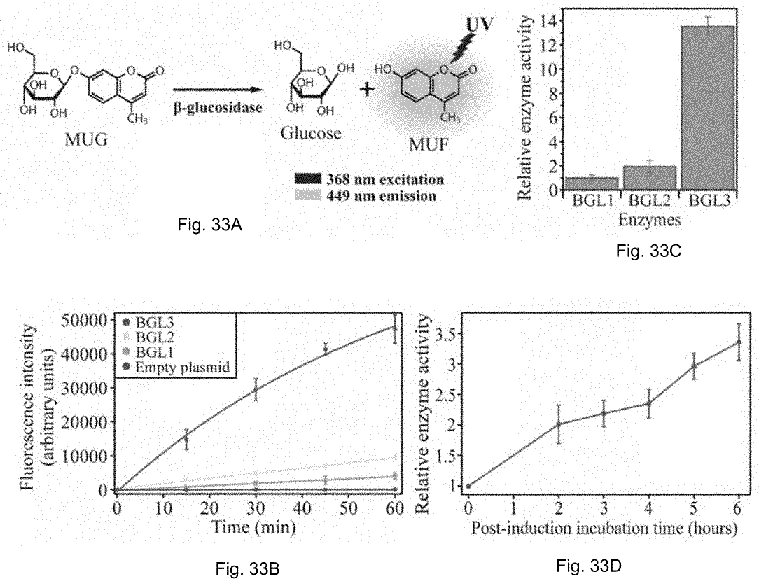

Expression optimization: screening active BGL enzymes. Referring to FIGS. 33A, 33B, 33C and 33D, there are shown expression optimization (single- and multi-point) assay to discover highly active BGL. Referring to FIG. 33A, there is shown a chemical scheme showing the enzymatic hydrolysis of 4-methylumbelliferyl beta-D-glucopyranoside (MUG) to 4-methylumbelliferone (MUF) by a beta-glucosidase (BGL). Referring to FIG. 33B, there is shown activity of three different BGLs in the presence of 2 mM MUG measured by fluorescence intensity (λₑₓ, lambda(ex)=369 nm and 2 km, lambda(em)=449 nm) over 60 min. Referring to FIG. 33C, there is shown a comparison of the rates of activity for the three enzymes relative to the lowest (BGL1). Referring to FIG. 33D, there is shown an induction profile of BGL3 over 6 h on the AIMS. For FIGS. 33B-33D, error bars represent +/−1 standard deviation across triplicates.

Given the versatility of the AIMS of this example, it is designed to analyze protein expression of more complex biological systems. For example, there has been a surge of interest in discovering enzymes for breaking down large sugar polymers (e.g., hexose and pentose sugars) that can be fermented into biofuels as potential substitutes for gasoline, diesel, and jet fuel. One group of enzymes, beta-glucosidases (BGL) have attracted considerable attention in recent years due to their ability to hydrolyze cellulose to produce glucose. Typically, BGL activity is first measured using artificial substrates such as 4-methylumbelliferyl beta-D-glucopyranoside (MUG). Hence, the AIMS was used in this example to investigate the catalytic activity of three BGLs based on the artificial substrate MUG (see FIG. 33A for chemical scheme).

To start, three reagent reservoirs were dedicated to the dispensing of multiple reagents (substrate, lysis solution, and stop solution) and 32 actuation electrodes to moving and mixing reagents with the induced culture, and five assay regions to measuring enzyme activity on device. After four hours of induction at 37° C., the cells were lysed and mixed with droplets containing the fluorogenic substrate MUG. Here, fluorescence over time was used as a read-out for enzyme activity. Many other possible probes or proteins relying on fluorescence are also compatible with the AIMS as shown in this example.

Fluorescence intensity for the enzymatic assay was measured directly on the device using a benchtop scanning well-plate reader and the enzyme activity curves are shown in FIG. 33B. As expected, there is an increase in the fluorescence measured over time for the three different BGL enzymes while little or no activity is observed in the negative control (i.e., an “empty” plasmid that does not contain any BGL). Specifically, in the single-point induction assay, the rate of activity measured by fluorescence was nearly identical for BGL1 and BGL2, but was significantly higher for BGL3. In fact, this rate is at least six times higher for BGL3 compared to the other two BGLs (FIG. 33C). To further optimize the activity of BGL3, a multi-point induction assay was performed to determine the optimal post-induction incubation period for BGL3 expression (pre-lysis).

As shown in FIG. 33C, the BGL3 showed highest expression (at least three times higher) after 6 h of induction and incubation compared to immediate induction and lysis (0 h). This is expected as the effect of post-induction incubation period affects the overall folding, accumulation and productivity of recombinant proteins in E. coli and therefore longer incubation times (>1 h) are more favorable. As for the high activity of BGL3 (compared to the other tested BGLs) it is not well understood, however, some groups have hypothesized that higher salt concentrations (and at neutral pH 7.0) will induce higher activity of enzymes and faster growth for thermo-tolerant organisms like Rhodothermus marinus. Furthermore, these organisms typically live in harsh environments and are required to constantly maintain their high-level thermostability and enzyme activity. Therefore, it is not a surprise that these enzymes can maintain their function and activity in a standard environment (at room temperature, constant pH, etc.). Regardless, these results confirm that the AIMS in this example is capable of automating induction and discovering enzymes that are possible candidates for biomass hydrolysis. The system described here may also be useful in testing a variety of enzymes to identify more candidates for biofuel production and synthetic biology applications.

The first automated induction microfluidics platform is presented in this example to monitor gene expression for synthetic biology applications using digital microfluidics. The AIMS allows on-device OD reading, in-line bacterial culture and induction in droplet format, and analysis of enzyme expression and activity. The system is characterized by optimizing the OD measurement and the growth conditions for bacterial cell culture. The AIMS of this particular example had a limit of detection of 0.035 OD units and was able to monitor bacterial growth at the micro-scale with no manual intervention over five hours. Additionally, the induction of an Rfgene in a pET expression vector is tested using different IPTG concentrations to generate a dose-response curve and compared it to the macro-scale experiment and found differences in their ultrasensitivity. Finally, the AIMS was used to measure the activity of three BGL enzymes directly on device after automated induction and optimized the highest active enzyme with different post-induction incubation conditions to optimize end-point activity. These results suggest the great potential for the application of digital microfluidics to automate induction and to analyze enzyme activity.

Supplementary Information is shown below and includes the following. Description of the fabrication procedure of the 3D enclosure with a figure showing the multiple layers of the AIMS. FIG. 2 shows the fabrication of the 3D enclosure for the AIMS. It has four layers (top to bottom): Layer 1 to hold the LED, Layer 2 is to support the pogo pin board that will apply electric potentials to the device, Layer 3 is used to support the device in place and Layer 4 is to position the sensor directly below the device.

A summary of each step is shown below for testing 5 conditions.

For the preparation of the starter culture, an overnight culture of the transformed E. coli BL21(DE3) cells in LB Amp was diluted to OD 0.1 in 150 mL of fresh media (2 min; 1 pipetting step per flask).

Frequent OD readings were taken to monitor growth and involved taking a 1 mL sample of the culture and measuring OD against a blank of LB at 600 nm (10 min; 1 pipetting step per reading and 1 for the blank).

Induction was carried out by adding 150 microliters of 1M IPTG to the culture flask (0.5 min; 1 pipetting step per flask) The induced culture was sampled at different times after induction by removing 1 mL samples from the growing flask and check OD (10 min; 5 pipetting steps per flask).

Lysis was done by adding 1 mL of lysis solution to each sample and leaving at room temperature for 15 min (2 min of hands-on time; 1 pipetting step per sample).

The assay was started by adding 50 microliters of lysate and 130 microliters of substrate solution to individual wells of a 96-well plate (10 min; 2 pipetting step per sample). It was stopped by the addition of 20 microliters of stop solution (1 pipetting step per sample).

AIMS was prepared by washing with EtOH and drying (10 min).

For the preparation of the starter culture, an overnight culture of the transformed E. coli BL21(DE3) cells in LB Amp was diluted to OD 0.1 in 1 mL of fresh media with 0.05% Pluronics F-68 (1 min; 3 pipetting steps).

Before starting the experiment, a droplet of starter culture, LB and IPTG were pipetted onto the device (1 min; 3 pipetting steps).

All subsequent OD readings and sampling of the induced culture are automated and do not require pipetting. (5 min to setup software.) In preparation for the assay, a droplet of lysis solution, substrate solution and stop solution were pipetted onto the device and actuated to their reservoirs (1 min; 3 pipetting steps).

All mixing steps for the assay are automated and do not require manual pipetting steps.

For 100 conditions, it is estimated for 4 different cultures that are interrogated with 5 different IPTG concentrations and 5 additive concentrations for the macro- and micro-scale. For the macroscale, cultures were started in flasks and then aliquoted into 96 well-plates. For the chip, the culture, buffers for dilutions, lysis, substrate, and stop solutions required refilling of the reservoir, hence the higher number of pipetting steps.

For 1000 conditions, it is estimated for 4 different cultures that are interrogated with 5 different IPTG concentrations and 50 additive concentrations. Pipetting steps were scaled linearly from 100 conditions while hands-on time are generally 3× more while the chip has been scaled linearly.

More information about the conditions on the chip are provided in Tables 9 and 10. Table 9 shows operating conditions on the chip according to some examples. Table 10 also shows operating conditions on the chip according to other examples.

TABLE 9
5 conditions on 1 chip

| | | | AIMS | | |
|---|---|---|---|---|---|
| Macro-scale | | | | | Pipetting |
| Step | Solution | Pipetting required | Step | Solution | required |
| Culture preparation | Overnight culture | 1 | | LB media | |
| | LB blank | 1 | Culture preparation | Overnight | 1 |
| OD readings | 4 OD readings | 4 | | culture | 1 |
| Induction | IPTG | 1 | | Pluronics | 1 |
| Sampling the induced culture | Culture | 5 | Setup on device | Diluted culture | 1 |
| aliquols | | 5 | | LB blank IPTG | 1 |
| Lysing each aliquot | Lysis solution | 5 | | Lysis buffer | 1 |
| | Transfer to 5 wells | 5 | Adding assay reagents | Substrate solution | 1 |
| Assay in 96-well plate | Substrate solution | 5 | | Stop solution | 1 |
| | Stop solution | | | | |
| Total | | 32 | Total | | 9 |

100 conditions on 1 chip

| Macro-scale | | | AIMS | | |
|---|---|---|---|---|---|
| Step | Solution | Pipetting required | Step | Solution | Pipetting |
| Culture preparation | Overnight culture | 4 | | LB media | 4 |
| | LB blank | 1 | Culture preparation | Overnight cuiture | 4 |
| OD readinos | 4 OD readiness per flask | 16 | | Pluronics | 4 |
| | Aliquot culture into 96-plate | 100 | Setup on device | Diluted culture | 20 |
| 5 induction concentrations | Adding to plate | 100 | | LB blank | 1 |
| (for each culture) | | | | | |
| 5 additive concentrations (for | Adding to plate | 100 | | IPTG | 1 |
| each culture) | | | | | |
| Lysing each aliquot | Lysis solution | 100 | | Additive | 1 |
| Asssy in 96-well plate | Substrate solution | 100 | | Butter for dilutions | 40 |
| | Stop solution | 100 | Adding assay reagents | Lysis buffer | 20 |
| | | | | Substrate solution | 20 |
| | | | | Stop solution | 20 |
| Total | | 621 | Total | | 135 |

1000 conditions on 10 chips

| Macro-scale | | | AIMS | | |
|---|---|---|---|---|---|
| Step | Solution | Pipetting required | Step | Solution | Pipetting |
| Culture preparation | Overnight culture | 4 | Culture preparation | LB media | 4 |
| | LB blank | 1 | | Overnight culture | 4 |
| OD readings | 4 OD readings per flask | 16 | | Pluronics | 4 |
| | Aliquot culture into 96-plate | 1000 | Setup on device | Diluted culture | 200 |
| 5 induction concentrations | Adding to plate | 1000 | | LB blank | 1 |
| (for each culture) | | | | | |
| 50 additive concentrations | Adding to plate | 1000 | | IPTG | 1 |
| (for each culture) | | | | | |
| Lysing each aliquot | Lysis solution | 1000 | | Additive | 1 |
| Assay in 96-welt plate | Substrate solution | 1000 | | Buffer for dilutions | 400 |
| | Stop solution | 1000 | Adding assay reagents | Lysis buffer | 200 |
| | | | | Substrate solution | 200 |
| | | | | Stop solution | 200 |
| Total | | 6021 | Total | | 1215 |

TABLE 10
5 conditions on 1 chip

| Macro-scale | | AIMS | |
|---|---|---|---|
| Step | Time min) | Step | Time (min) |
| Starter culture dilution | 2 | Chip preparation | 10 |
| Taking 4 × 1 OD readings; | 10 | Starter culture dilution | 1 |
| plate reader setup | | | |
| | | Automation system setup | 5 |
| Inducing 1 culture | 1 | Reagent setup | 1 |
| Taking 5 aliquots of the | 10 | Assay setup | 1 |
| culture | | | |
| Cell lysis | 2 | | |
| Assay setup | 10 | | |
| Total | 35 | Total | 18 |

100 conditions on 1 chip

| Macro-scale | | AIMS | |
|---|---|---|---|
| Step | Time min) | Step | Time min) |
| Starter culture dilution (into 4 flasks) | 10 | Chip preparation | 10 |
| Taking 4 × 4 OD readings | 40 | Starter culture dilution (incl. | 10 |
| readings; plate reader setup | | multiple cultures) | |
| Generating 5 inducer | 40 | Automation system setup | 10 |
| concentrations and inducing | | | |
| Generating 5 additive | 40 | Reagent setup | 5 |
| concentrations and adding to | | | |
| culture | | | |
| Cell lysis | 40 | Assay setup | 15 |
| Assay setup | 200 | | |
| Total | 370 | Total | 50 |

1000 conditions on 10 chips

| Macro-scale | | AIMS | |
|---|---|---|---|
| Step | Time (min) | Step | Time (min) |
| Stater culture dilution | 10 | 10 chip preparation | 15 |
| (into 4 flasks) | | (can wash in parallel) | |
| Taking 4 × 4 OD readings | 40 | Starter culture dilution | 10 |
| readings; plate reader setup | | (1 time) | |
| Generating 5 inducer | 40 | Automation system setup | 10 |
| concentrations and inducing | | (1 time) | |
| Generating 50 additive | 120 | Reagent setup (10 times) | 50 |
| concentrations and adding | | | |
| to culture | | | |
| Cell lysis | 120 | Assay setup (10 times) | 150 |
| Assay setup | 600 | | |
| Total | 930 | Total | 235 |

According to another example, a summary of each step is shown below:

For the preparation of the starter culture, an overnight culture of the transformed E. coli BL21(DE3) cells in LB Amp was diluted to OD 0.1 in 150 mL of fresh media (2 min; 1 pipetting step per flask).

Frequent OD readings were taken to monitor growth and involved taking a 1 mL sample of the culture and measuring OD against a blank of LB at 600 nm (10 min; 1 pipetting step per reading and 1 for the blank).

Induction was carried out by adding 150 mL of 1 mM IPTG to the culture flask (0.5 min; 1 pipetting step per flask) The induced culture was sampled at different times after induction by removing 1 mL samples from the growing flask and check OD (10 min; 5 pipetting steps per flask).

Lysis was done by adding 1 mL of lysis solution to each sample and leaving at room temperature for 15 min (2 min of hands-on time; 1 pipetting step per sample).

The assay was started by adding 50 microliters of lysate and 100 microliters of substrate solution to individual wells of a 96-well plate (10 min; 2 pipetting step per sample). It was stopped by the addition of 50 microliters of stop solution (1 pipetting step per sample).

AIMS was prepared by washing with EtOH and drying (10 min).

For the preparation of the starter culture, an overnight culture of the transformed E. coli BL21(DE3) cells in LB Amp was diluted to OD 0.1 in 1 mL of fresh media with 0.05% Pluronics F-68 (1 min; 3 pipetting steps). Before starting the experiment, a droplet of starter culture, LB and IPTG were pipetted onto the device (1 min; 3 pipetting steps).

All subsequent OD readings and sampling of the induced culture are automated and do not require pipetting. (5 min to setup software.) In preparation for the assay, a droplet of lysis solution, substrate solution and stop solution were pipetted onto the device and actuated to their reservoirs (1 min; 3 pipetting steps).

All mixing steps for the assay are automated and do not require manual pipetting steps.

### Example 5

This example describes an automated microfluidic gene-editing platform for deciphering cancer genes, in accordance with another embodiment of the invention.

Gene-editing techniques such as RNA-guided endonuclease systems are becoming increasingly popular for phenotypic screening. Such screens are normally conducted in arrayed or pooled formats. There has been considerable interest in recent years to find new technological methods for conducting these gene-editing assays. It is reported here the first digital microfluidic method that can automate arrayed gene-editing in mammalian cells. Specifically, the method in this example was useful in culturing lung cancer cells for up to six days, as well as implementing automated gene transfection and knockout procedures. In addition, in this example, a standardized imaging pipeline to analyze fluorescently labelled cells was also designed and implemented during these procedures. A gene editing assay for interrogating the MAPK/ERK pathway was performed to show the utility of the platform and to determine the effects of knocking out the RAF1 gene in lung cancer cells. In addition to gene knockout, the cells were also treated with an inhibitor, sorafenib tosylate, to determine the effects of enzymatic inhibition. The combination of enzymatic inhibition and guide targeting on device resulted in lower drug concentrations for achieving half-inhibitory effects (IC50) compared to cells treated only with the inhibitor, confirming that lung cancer cells are being successfully edited on the device. It is expected that the system in this example will be useful for other types of gene-editing assays and applications related to personalized medicine.

Recent efforts in cancer characterization are shifting towards a more personalized approach rather than hierarchical classifications based on chemosensitivity experiments. Cancer is a heterogeneous disease that highly differs in genetic makeup and relies on different pathways for survival, which gives rise to a wide-range of potential responses to different anti-cancer agents. One method that has been rapidly growing in interest is to use CRISPR-based screens to systematically identify the genes that are required for the survival and proliferation of mammalian cells. Such a method allows complete and permanent inactivation of genes and can offer insight into the genetic basis of the disease and lead to the identification of new drug targets. Several groups have reported successful editing of endogenous genes in cells in culture via transfection of plasmid DNA or stable delivery into cells through the use of lentiviruses or other retroviruses. These systems contain the Cas9 which can be targeted to specific location in the genome by a single guide RNA that complements the target DNA and be used for loss-of function screens aimed at identifying potential drug targets for cancer treatment.

The most common format for these loss-of-function perturbations is in vitro “pooled” screens relying on the delivery of Cas9 nucleases and a “pool” of guide RNAs (sgRNAs) into the cells by transfection or transduction. Pooled libraries allow screens that simultaneously assess the effect of knocking out hundreds to thousands of individual genes at multiple loci in a phenotypic readout, such as proliferation or metastasis assays. Although such developments provide new opportunities for drug target identification and validation, interpretation of results in a pooled format rely on differential representation of guide RNAs after vs before (as assessed by Next-Generation Sequencing) and rely on enrichment of multiple guide RNAs as a validation of target relevance. Furthermore, the complexity of population dynamics, each cell being in competition with many others, may contribute to biases resulting in higher relative abundance of some perturbations compared to some others. An alternative to “pooled” screens is to implement “arrayed” screens where cells are genetically perturbed only with one known gene target. This can potentially allow use of a wider range of cellular phenotypes to be investigated. Limitations of arrayed experiments are the associated costs (usually an order of magnitude more expensive than pooled libraries) since they require special facilities that use automation for the handling of plates and the inefficient workflow that includes labor-intensive preparatory work to build and produce individual guide libraries and transferring the samples to other platforms for analysis. Thus, an automated and integrated platform that will culture cells for days, allow efficient handling of mammalian cells and reagents, express the gene editing machinery targeting an individual gene or locus in cells, and assay cell phenotypes will be beneficial for these arrayed-type experiments to save overall costs and to improve the workflow that minimizes the time frame between perturbation and measurement. This is described in this example.

Arrayed libraries are typically generated in multi-well plates, where each well contains a virus or vector, or reagents with a guide targeting a specific gene. The tools used for these types of experiments, such as automated robotics coupled with flow cytometry, can provide an exploration of complex phenotypes arising from single perturbations. Despite their outstanding features in reducing cell death or limiting off-target mutagenesis associated with editing, these techniques suffer from three key limitations. First, available liquid handling technologies, data acquisition equipment and data storage/processing systems have traditionally been expensive and have large footprints that are well outside of the budgetary reach of many laboratories. In addition, the programming software packages are not standardized between laboratories which frequently discourages inter-disciplinary scientists and researchers to use robots as it usually requires more time and effort to instruct a robot to perform a task. Second, liquid handlers for cell culture and sample preparation have multiple sources of variability (especially at the nL volumes) which can cause unintended perturbations related to the gene-editing process, e.g., different volumes can alter cell growth resulting in unequal number of cells across wells of a plate. This can pose variability issues with downstream analysis in terms of measuring transfection and knockout efficiencies related to cell density. Third, there is a lack of standardization in assay and in instrument set-up for flow cytometry and especially for how flow data are analyzed and reported. Thus, these approaches may present additional challenges to the already complex procedures of gene editing.

A strategy to alleviate the challenges described above is to use flow-based microfluidics and fluorescent microscopy techniques. The development and maturation of these microdevices and optical techniques have been a boon to be used for cell-based assays and genomics. Microfluidics allows the manipulation of small volumes of liquids in nanoliter (or smaller) scales in interconnected micron-sized dimension channels and allows the automated delivery of chemical stimulant to cells. The resulting cellular responses can be imaged with fluorescent reporters or fluorescent labelling techniques. For gene-editing assays, this includes delivery of Cas9 into the cells and visualizing them via a fluorescence reporter or using flow cytometry techniques to determine if the Cas9 has been delivered into the cell. These methods offer an exciting new framework into gene-editing, but do not incorporate two key steps in the gene-editing process. First, the serial nature of flow-based microfluidics present challenges in delivering many reagents (e.g., lipids, DNA, culture medium, drugs, etc.) needed for the gene-editing process. Indeed, valves can be integrated into the PDMS-based microdevice, but this can be very complicated to setup (in terms of alignment and insertion of tubing) and to operate. Second, two key steps in gene editing—cell culturing and analysis—are typically been performed off-chip, e.g., the cells have been cultured in flasks and analyzed by flow cytometry. A standardized automated gene-editing platform that can automate all the steps would improve the workflow.

To address the challenges described above, it is reported here in this example a new droplet-based method for gene editing called microfluidic Automated CRISPR-Cas9 Editing (ACE). As shown in this example, this can be used to automate all the steps for gene-editing, e.g., culture, delivery, and analysis. In this example, it is reported the application of ACE to evaluate the well-characterized mitogen-activated protein kinase or extracellular signal-regulated kinase (MAPK/ERK) pathway and including downstream editing of the Raf-1 gene with and without the Raf-1 inhibitor sorafenib tosylate. The results recapitulate what is known about the pathway and its effect on cell viability, but the technique presented here in this example shows that it is possible to conduct an automated gene-editing workflow from cell culturing to analysis with an open-source automation system coupled with a standardized pipeline to analyze the transfected/knockout fluorescent cells. These results are the first of their kind and serve as examples of what is possible for the future-new techniques for probing other types of cancer, or other indications. This may serve as a platform for ex vivo applications, e.g., those relating to personalized medicine that require automated cell culture, transfection, CRISPR-Cas9 editing, and drug inhibition, etc.

Device fabrication and assembly, automation setup and operation is described herein.

Reagents and Materials. Microfluidic device fabrication reagents and supplies included chromium-coated glass slides with S1811 photoresist from Telic (Valencia, Calif.), indium tin oxide (ITO)-coated glass slides, R_{S}=15-25 ohm (cat no. CG-61IN-S207, Delta Technologies, Loveland Colo.), FluoroPel PFC1601V from Cytonix LLC (Beltsville, Md.), MF-321 positive photoresist developer from Rohm and Haas (Marlborough, Mass.), CR-4 chromium etchant from OM Group (Cleveland, Ohio), AZ-300T photoresist stripper from AZ Electronic Materials (Somerville, N.J.), DuPont AF from DuPont Fluoroproducts (Wilmington, Del.). Transparency masks for device fabrication were printed from CADArt (Bandon, Oreg.) and polylactic acid (PLA) material for 3D printing were purchased from 3Dshop (Mississauga, ON, Canada). General chemicals for tissue culture were purchased from Wisent Bio Products (Saint-Bruno, QC, Canada). Invitrogen Lipofectamine 3000 Transfection Reagent was purchased from Thermo Fisher Scientific (Waltham, Mass.). Unless specified otherwise, general-use chemicals and kits were purchased from Sigma-Aldrich (St. Louis, Mo.). Plasmids for this study were purchased from Addgene or donated (see Tables 11 and 12) and primers were purchased from Invitrogen (Waltham, Mass.), and genes (438 bp) were synthesized by IDT (Coralville, Iowa). Sorafenib Tosylate was purchased from Selleckchem (Houston, Tex.).

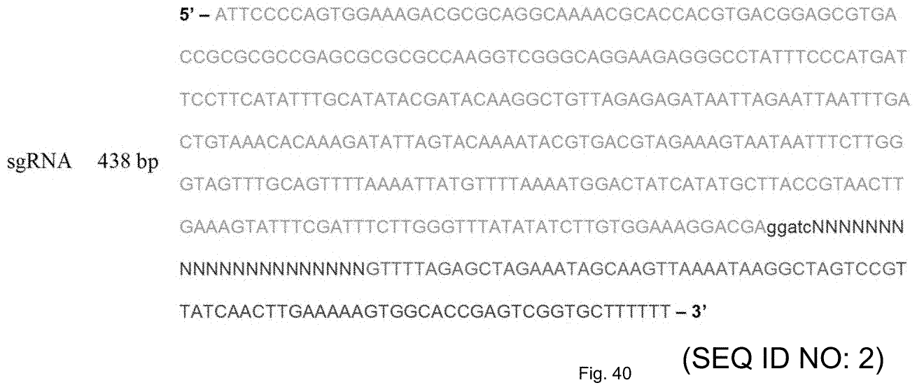

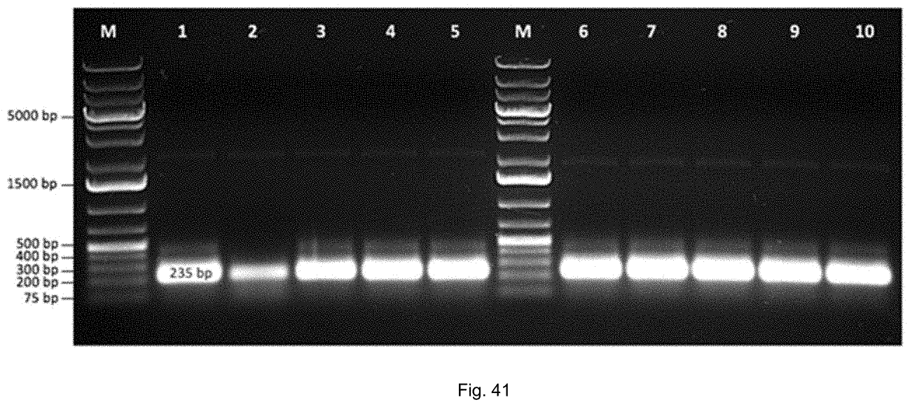

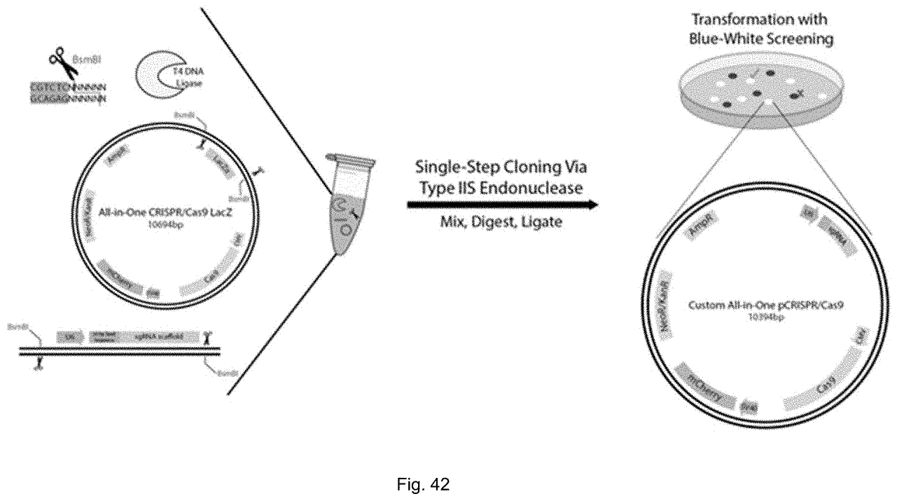

Plasmid construction and purification. CRISPR guide RNAs (gRNA) were synthesized (FIG. 40; see SEQ ID NO: 2) by IDT Technologies after being designed via the Benchling online platform, and were PCR amplified to create g-blocks flanked with Esp3I type IIS restriction sites (see Table 13 for primers). Individual PCR reactions used 10 microliters 5× Phusion buffer, 1 microliter dimethylsulfoxide (DMSO), 20 ng template DNA, individual dNTPs and primers to a final concentration of 200 micromolar and 0.5 micromolar each, 0.5 microliters Phusion polymerase and distilled water up to 50 microliters. The following PCR thermocycling conditions were used: initial denaturation at 98° C. for 30 s followed by 35 cycles of denaturation at 98° C. for 10 s, annealing at 55° C. for 30 s and extension at 72° C. for 30 s/kb, and a final extension step at 72° C. for 10 min. PCR products were loaded into a 0.8% agarose gel in TAE buffer and resolved at 130 V for 30 min. The corresponding bands from a gel (FIG. 41) were extracted using a gel extraction kit from BioBasic (Markham, ON, Canada). The one-step gRNA cloning method was adapted from the Findlay et al. protocol (Findlay, et al., “A Digital PCR-Based Method for Efficient and Highly Specific Screening of Genome Edited Cells,” PLoS One, 11(4):e0153901, 2016). The gRNAs were assembled via restriction digestion/ligation into the All_in_one_CRISPR/Cas9_LacZ backbone containing Esp3I cut sites on both the 3′ and 5′ ends of LacZ-alpha gene fragment. Individual reactions used 25 ng of the g-Block (10 ng/microliter), 75 ng All_in_one_CRISPR/Cas9_LacZ1 microliters BsmBI (10 U/microliter), 1 microliter T4 ligase (Thermo Fisher, Waltham, Mass.), 2 microliters T4 buffer and nuclease-free water to 20 microliters total. The mixture was incubated in a thermal cycler at 37° C. for 5 min, 16° C. for 10 min, 37° C. for 15 min and 80° C. for 5 min. Assembled products were heat-shock transformed into the LacZ-alpha deficient DH5-alpha E. Coli strain. The transformed products were grown on LB/S-Gal agar blend and assembled products were discriminated by a color bias for colonies-blue colonies contained the LacZ-alpha fragment required for S-Gal hydrolysis, whereas white colonies possessed the g-block insert (i.e., without the LacZ-alpha gene). White colonies were picked and grown overnight before being DNA purified and sent out for sequencing by Eurofins Genomics (Toronto, ON, Canada) (see FIG. 42 for a schematic of the procedure). All constructed plasmids were deposited to the online Addgene repository (Cambridge, Mass.).

Macro-scale cell culture, transfection, and knockout. Human lung squamous cell carcinoma dual-labeled stable NCI-H1299 cell line was purchased from Genecopoeia, Inc (SL001, Rockville, Md.). H1299 cells were grown in RPMI 1640 containing 10% fetal bovine serum with no antibiotics in an incubator at 37° C. with 5% CO₂.

For macroscale transfection experiments, cells were seeded (1.0×10⁵ cells/mL) a day before transfection (day 0) to reach 70-80% confluency in 24 well-plates. On day 1, 500 ng/microliters of DNA were pre-mixed with 1 microliter of P3000 reagent in 25 microliters of Opti-MEM and added to 1.5 microliters Lipofectamine 3000 that was pre-mixed in 25 microliters Opti-MEM. Lipids were then incubated with the DNA at room temperature for 10 min to form lipid-DNA complexes. The complexes were pipetted into each individual well containing the adhered cells. On day 2, after incubation, the lipid complex with DNA was removed by aspiration and fresh complete media was replenished into the wells. Cells were stained with Hoechst 33342 and incubated for 30 min on day 3. The cells were imaged with a 20× objective on an Olympus IX73 inverted microscope (Olympus Canada, Mississauga, ON, Canada) that has fluorescence imaging capabilities (Hoechst: λₑₓ, lambda(ex)=350 nm and λₑₘ, lambda(em)=461 nm; GFP: λₑₓ, lambda(ex)=488 nm and λₑₘ, lambda(em)=509 nm; mCherry: λₑₓ, lambda(ex)=585 nm and λₑₘ, lambda(em)=608 nm). Fluorescence images were further analyzed using the CellProfiler transfection pipeline.

For knockout experiments, the cell seeding followed the steps described in the transfection experiments. For transfection (day 1), 600 ng/microliter of assembled pCRISPR plasmid (with the inserted sgRNA) were mixed with the same reagent compositions as above (1:10 ratio of lipid complexes to media in wells). After cells were maintained (replaced with fresh media) on day 3, cells were sub-cultured at a 1:4 ratio in a new 24-well plate on day 4 by washing the cells with 200 microliters of PBS and removing the cells with 150 microliters of 0.25% trypsin-EDTA. Following further maintenance on day 5, on day 6 the cells were stained with 1 micromolar Hoechst 33342 and imaged using the same microscope (and filters) for knockout analysis using the CellProfiler knockout pipeline. Data were tested at P<0.05 for statistical significance using a student's t-test.

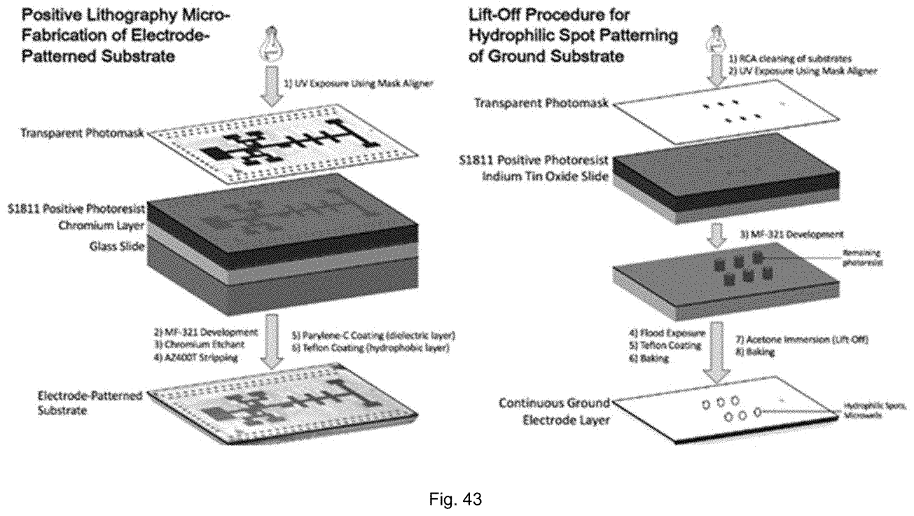

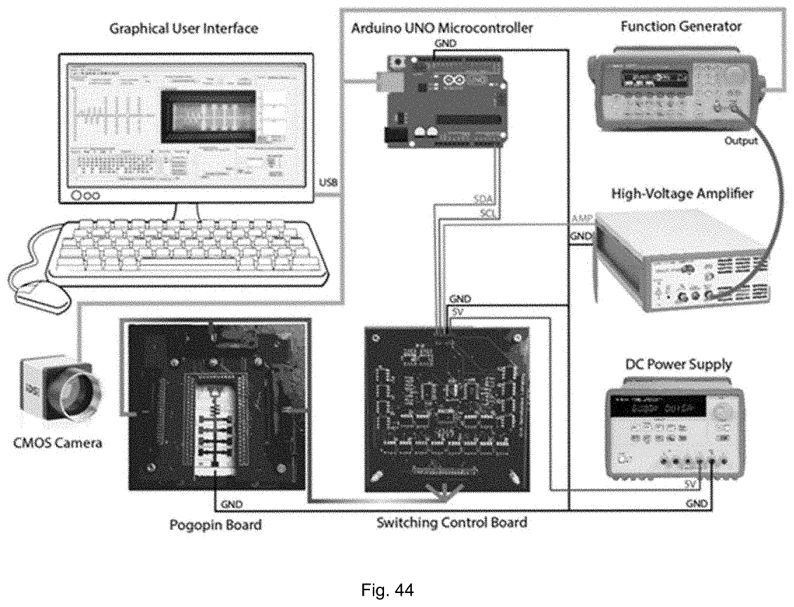

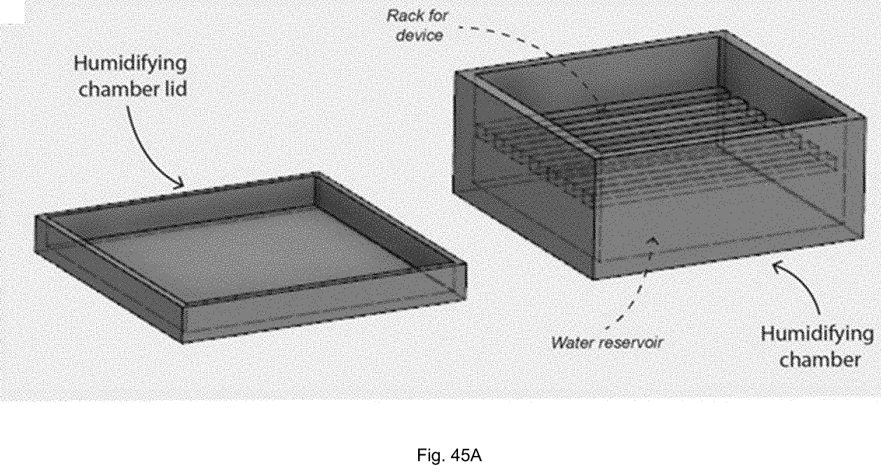

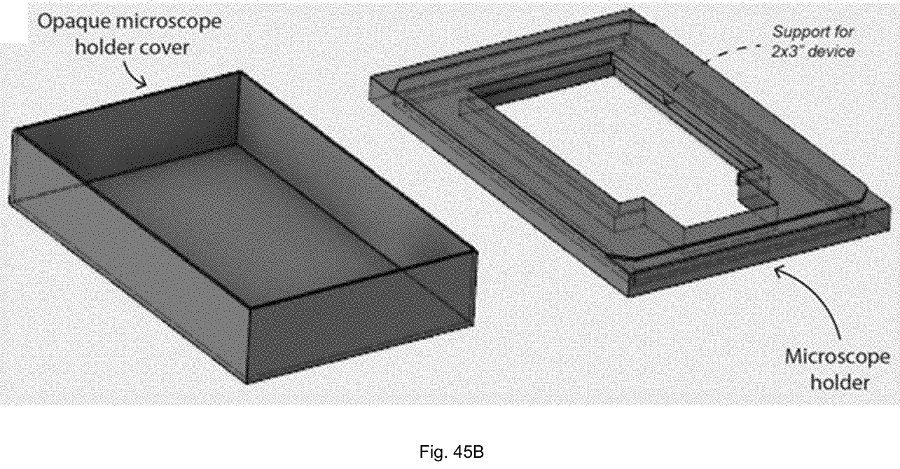

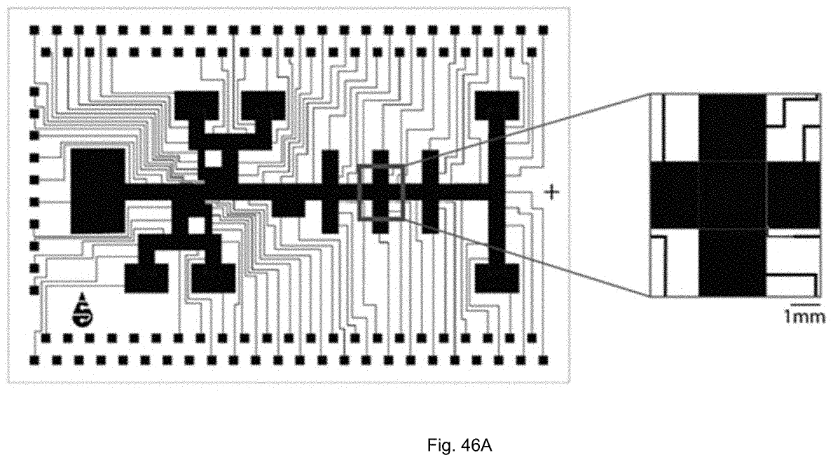

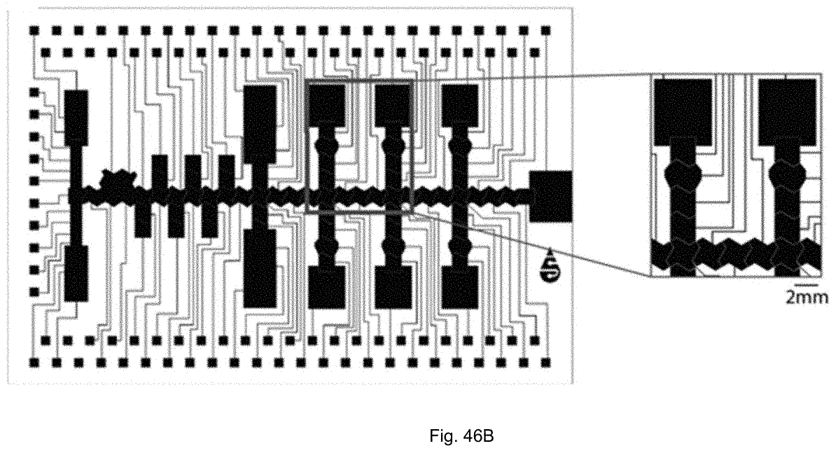

Microfluidic cell culture, transfection, and knockout. DMF was used to automate the protocols required for gene editing including cell seeding, culture, lipid transfection, reagent delivery, staining, washing, and drug inhibition (see FIG. 43 for fabrication procedure, FIG. 44 for automation system). In all droplet manipulation steps, the device was oriented in standard configuration, with the top plate on top, while in all incubation steps, the devices were inverted, with the top plate on the bottom and in a 3D-printed humidified chamber (FIG. 45A). Before seeding cells onto DMF devices (day 0), cell cultures were grown in T-75 flasks and were rinsed with PBS, trypsinized and suspended in 10 mL of complete media. After centrifugation at 1,000×g for 5 min, the cell pellet was suspended in 2 mL of complete media (and supplemented with 0.05% w/v Pluronics F-68) such that the initial concentration of cells is ˜1.5×10⁶ cells/mL.

To seed and culture cells (day 0), 2 microliters of cells at 1.75×10⁶ cells/mL in culture medium were pipetted onto the edge of the ITO and actively dispensed from the reservoirs into 690 nL unit droplets. These droplets were sequentially passively dispensed on each vacant lift-off spot forming 285 nL droplets on the hydrophilic sites. The excess liquid from the spot was actuated to a waste reservoir and removed with a KimWipe. The device was inverted and incubated in a 37° C. incubator with 5% CO₂ overnight allowing the cells to adhere onto the hydrophilic spot. In the next 7 steps, a sequence of transfection reagents was mixed to form lipid complexes and delivered (via passive dispensing) to each hydrophilic site that contains cells on day 1. (1) 1 microliter of Lipofectamine was diluted in 25 microliters of Opti-MEM and premixed and 2 microliters was added to a reservoir. (2) 500 ng/microliter of the plasmid DNA to be inserted and 1 microliter of P3000 reagent diluted in 25 microliters of Opti-MEM was also added to another reservoir. (3) Both reagents were actively dispensed (360 nL each), merged and mixed in a square configuration using 2×2 electrodes and incubated for 10 min to form lipid complexes. (4) The lipid complexes were diluted in a 1:1 ratio by combining with a 690 nL unit droplet of Opti-MEM. (5) After mixing, the complexes were delivered to the cells via passive dispensing 6×285 nL and incubated for 24 h overnight. (6) The lipid complexes on the cells were removed by passively dispensing 6×285 nL of fresh complete media. (7) After 24 h, 6×285 nL of 1 micromolar Hoechst stain in liquid media was passively dispensed to each well and fluorescence images were acquired to measure transfection efficiency. In transfection optimization experiments, lipid:media ratios in step 4 were changed by performing serial dilutions, e.g., by splitting the initial droplet containing the 1:1 diluted complexed DNA into two daughter droplets (360 nL each) and mixing it with a unit droplet of liquid media (690 nL). mCherry transfection efficiency was monitored on the device by microscopy, mounting the devices on a custom 3D-printed microscope holder (FIG. 45B). Fluorescence images were further analyzed using the CellProfiler transfection pipeline.

For assessing GFP knockout efficiency, 2 microliters of cells (˜1.75×10⁶ cells/mL) were pipetted onto the reservoir and a unit droplet was actuated to the vacant lift-off spot. After overnight incubation, the adhered cells were transfected with 600 ng/microliters of pCRISPR (with the inserted sgRNA) following the steps for transfection (steps 1-6). Cells were maintained until day 5 by passively dispensing fresh media daily (6×285 nL) to each cell culture site. GFP knock-out was monitored on the device by using microscopy and mounting the devices on a custom 3D-printed microscope holder to ensure healthy cells during image acquisition. On day 5, the microwells were rinsed with PBS followed by 0.25% trypsin-EDTA by passively dispensing a unit droplet across each well. Following incubation at 37° C. for 5 min, the top-plate was disassembled from the bottom-plate and 100 microliters of complete media was pipetted directly onto each hydrophilic spot and transferred to an individual well of a 96-well plate and incubated for 2 days. On day 6, 1 micromolar Hoechst stain in liquid media was added to each well and fluorescence images were acquired to measure knock-out efficiency using the custom CellProfiler knock-out efficiency pipeline.

Cell imaging and CellProfiler pipeline. Top plates bearing stained and fluorescent cells were analyzed using an inverted Olympus microscope. Typically, images were acquired using a Hamamatsu digital camera (Model C1140-42U) camera with the HC ImageLive software. Images were typically acquired using a UV (250 ms exposure time), GFP (500 ms), or mCherry filter set (1000 ms).

Images from the microscope were analyzed using the open-source CellProfiler 2.2.0 r9969F42 software package. A custom pipeline was developed, including image cropping, identifying individual and overlapping cells from Hoechst-stained and mCherry fluorescent images, counting total number of cells, measuring the size and shape of cells, creating binary images of the cells (i.e., black and white images), and comparing knocked-out and non-knocked out cells (UV and GFP channels). For transfection analysis, the pipeline is divided into four modules. In module 1, the software was instructed to smooth the Hoechst-stained image with a Gaussian filter (G, sigma=1) and uses the Otsu Global thresholding method to detect objects with diameters of 20-100 pixel units (two classes, threshold correction factor=0.8).

Neighboring pixels are grouped into objects and undesired clumped objects (e.g., two close overlapping objects) are declumped using intensity segregation. In module 2, the software was instructed to threshold the mCherry image to select cells that have the plasmid (threshold correction factor=1) and binarize the image to have black (corresponding to mCherry-negative) and white (mCherry-positive) regions. In module 3, the software was instructed to overlap images from module 1 and 2 where the image from module 2 served as a mask for the identified nuclei in module 1. All the nuclei-stained cells (from module 1) overlapping with an mCherry-positive region (module 2) were retained and counted which gave the total of transfected cells. In module 4, the equation 1 is used:

Efficiency (%)=[(overlapping nuclei)/(total nuclei)]×100%

The result corresponds to the proportion of mCherry-positive nuclei (e.g., transfected cells) versus the total number or nuclei. Each data point was further corrected from the negative control cells (e.g., non-transfected cells) using the same pipeline.

For the knockout pipeline, four similar modules were created to analyze knockout efficiencies. In module 1, the software followed the instructions for the transfection pipeline. In module 2, a GFP image was thresholded using the Otsu method (two classes, 0.65 threshold correction factor). Module 3 overlapped the image with the image from module 2 serving as a mask for the image from module 1. Nuclei-stained cells that overlap with GFP-positive cells (90% of its total pixels) were not considered as knocked-out cells. Module 4 followed equation 1, i.e., the total number of knocked out cells from module 3 divided by the total number of cells obtained from module 1 to obtain knockout efficiencies.

MAPK/ERK pathway experiments. The MAPK/ERK pathway experiments used two key components: CRISPR-Cas9 genomic disruption of Raf1 and drug inhibition using sorafenib tosylate. In the macroscale, 0.75×10⁵ cells/mL of H1299 cells were seeded on day 0 in 24-well plates. 600 ng of the pCRISPR plasmid targeting eGFP (control) or RAF1 was applied to the wells containing the cells on day 1. On day 3, drug conditions were added at different concentrations: 0 micromolar, 7.5 micromolar, 15 micromolar, 30 micromolar, 60 micromolar, 120 micromolar which were diluted in complete media. On day 5, 5 micromolar Calcein-AM violet stain (λₑₓ, lambda(ex)=408 nm and λₑₘ, lambda(em)=450 nm) diluted in 250 microliters fresh serum-free media was added to the cells and incubated at 37° C. for 30 min. The viability of cells was assessed by performing a fluorescence well scan using the CLARIOStar well-plate reader. The measured fluorescence was normalized to the control to determine the percent viability.

Similarly, in the microscale, the transfection protocol is followed for seeding cells and the 7-step protocol for transfection of the pCRISPR plasmid containing sgRNA targeting eGFP or Raf-1. The standard step 7 was replaced with step 7a and step 7b. In step 7a, sorafenib tosylate in complete media was actively dispensed into unit droplets and then diluted in liquid media to form six different concentrations (0 micromolar, 7.5 micromolar, 15 micromolar, 30 micromolar, 60 micromolar, 120 micromolar) of which one droplet (0.7 microliters) was used to passively dispense onto each hydrophilic spot and the other droplet was saved for future dilutions. After all cells were interrogated with the drugs, they were incubated for two days. In step 7b, six unit droplets of 5 micromolar Calcein-AM violet stain were passively dispensed to the cells and incubated for 30 min in which images were taken to count the cells using a single module imaging pipeline. Calcein-stained image was smoothed with a Gaussian filter (G, sigma=1) and used the Otsu Global thresholding method to detect objects with diameters of 20-100 pixel units (two classes, threshold correction factor=1.25). Neighboring pixels are grouped into objects and undesired clumped objects (e.g., two close overlapping objects) are declumped using intensity segregation. The counted cells were normalized to the control (e.g., cell interrogated with no drugs). All curves were fit with a sigmoid function and probed for statistical significance using an F-test in the linear region.

Digital microfluidic platform for gene-editing. There has been a wide variety of applications that use gene-editing techniques, particularly those involving silencing genes or developing gene therapy techniques related to diseases. Such applications would benefit from a miniaturized automated technique that is capable of integrating the gene-editing process on one platform. This example presents an automated CRISPR-based microfluidic platform that is capable of culturing, editing, and analysing cells. This platform presented in this example is called “ACE” after the function of this platform—Automated CRISPR Editing.

The ACE platform was developed to automate the processes related to gene-editing and to address the limitations in current techniques to evaluate genes related to a cancer pathway. ACE relies mainly on digital microfluidics (DMF) that will automate the gene-editing processes through its versatile liquid handling operations: dispense, merge, mix, and split droplets. This work builds upon several DMF and cell-culture studies that have established proof-of-principle protocols. This example illustrates the first DMF-based technique that is capable of cell culturing, gene editing, and image analysis for lung cancer cells, e.g., shown in FIGS. 34, 35 and 36.

Specifically, this platform was tailored to rapidly deliver single-guided RNAs (sgRNA) in an all-in-one pCRISPR plasmid format to effectively knockout targeted genes in lung cancer cells. The device was customized with reservoirs to hold necessary reagents for lipid-mediated transfection and designated regions for incubation, along with a cell culture region to accommodate cell seeding, maintenance, and transfection (FIG. 34). Genomic disruption can be assessed phenotypically on the same device using a microscopy-based imaging analysis workflow to determine plasmid delivery efficiencies through monitoring fluorescent protein expression and cell viability using various fluorescent dyes.

The device comprises of two parallel-plates separated by a 140 micrometer spacer. The bottom plate uses metal-patterned electrodes with dielectric and hydrophobic layers and serves to manipulate the droplets containing the constituents for gene-editing. One of the primary reasons for using DMF in this work is the individual addressability of droplets that allows for controlled automated liquid handling on the device. However, a continuous challenge with DMF is the reproducibility of droplet movement on the device, especially for liquids that are high in viscosity (e.g., complete cell media). To alleviate this challenge, there are studies that introduce chemical additives or an immiscible fluid to prolong droplet movement. However, in this example, it was initially observed that droplet movement of protein rich solutions (e.g., suspended cells) are difficult to move after two days of culturing and maintenance (see FIGS. 46A and 46B for designs). This is problematic given that typical gene-editing phenotypic readouts are usually observable beyond two days. Previous work has shown that changing the electrode shape can enhance the driving force of the droplet. Here, the electrode design in this example has been modified such that the boundary between electrodes are interlaced and have added chemical additives in the droplet. It is observed that droplet movement was improved and all the droplet movements necessary (˜300 total movements for five days) for cell culture and maintenance, and the gene editing assay were completed. As described in other studies, the primary reason for this improvement could be due to the overlap of the droplet on the adjacent electrode which increases the applied force on the droplet and thereby increases the velocity of the droplet movement. This will minimize the time a droplet is on activated electrode which can minimize biofouling on the hydrophobic surface and allows more actuations on the device.

The top-plate is responsible for adherent cell culture and relies on the microfabrication of six 1.5 mm diameter hydrophilic sites. Typically, the cells in suspension are manipulated by applying an electric potential. When moved across the hydrophilic spot, a fraction of the droplet remains pinned to the hydrophilic spot and will serve as the cell culture microvessel—this operation is called “passive dispensing” (FIG. 34, inset). The delivery of cells to these hydrophilic spots will allow cells to adhere, spread, and proliferate in an upside-down configuration (top plate on the bottom). To prevent evaporation, the devices were incubated in a 3D printed humidified chamber (FIG. 45A).

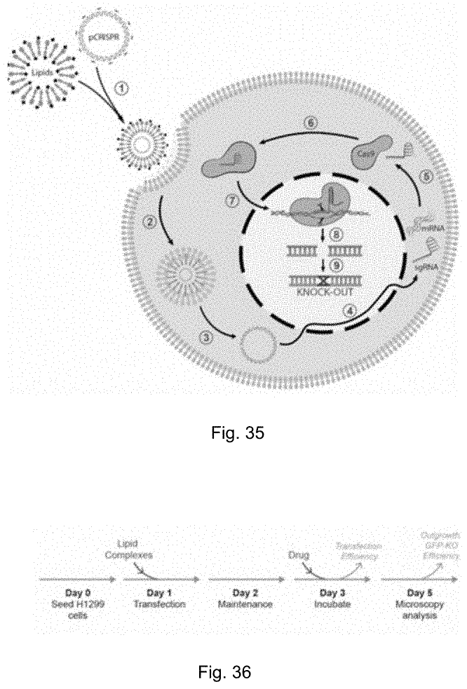

After the cells are fixed, the device is flipped to its standard configuration and at designated periods, the cells are transfected with CRIPSR-based plasmids that are complexed in lipid vesicles for efficient delivery of exogenous material to the cells. As shown in FIG. 35, successful gene-editing in individual cells using the method occur when cells co-express both the Cas9 and the sgRNA that assemble into a ribonucleoprotein (RNP) complex and is delivered to the nucleus for targeted cleavage. The complex will seek the target sequence, complementary to the seed sequence, using the designed sgRNA and will cleave the target DNA which results in a double stranded break and ideally causing a knockout. For downstream analysis, the cells are incubated and labeled with a fluorescent dye delivered in liquid media by passive dispensing to determine efficiencies of transfection and gene knockout. Using a custom 3D-printed microscope holder (FIG. 45B), images of the top plate containing cells (without disassembling the device) are captured which can be analyzed by CellProfiler to calculate the percentage of transfected or knocked-out cells to the total number of cells.

There have been previous studies which have cultured adherent cells with DMF, but this is the first time that lung cancer cells have been cultured, edited, and analyzed on such a platform. Using the passive dispensing technique, the reproducibility and viability of the lung cancer cells were tested on the hydrophilic spots. Factors such as cell seeding density and microwell culture volume are critical to the maintenance of the cell viability and morphology on the device. Cells were seeded at densities between 1 and 2×10⁶ cells/mL and maintained over five days by exchanging media once per 24 h to sustain viable lung cancer cells with appropriate morphologies. Depending on the assay, the seeding densities were altered to ensure cells are ready for the experiments. For example, for transfection optimization, cells were grown to be 70-80% confluent to ensure optimal transfection and therefore cells were seeded at a higher density—1.75×10⁶ cells/mL (see FIG. 36 for gene-editing assay timeline). For longer term experiments—such as knockout experiments which required 5-6 days—cells were seeded at a lower density to achieve the desired confluence for gene editing. At higher densities >1.5×10⁶ cells/mL, the cells reached confluency quickly, resulting in cell senescence prior to endpoint knock-out efficiency measurements.

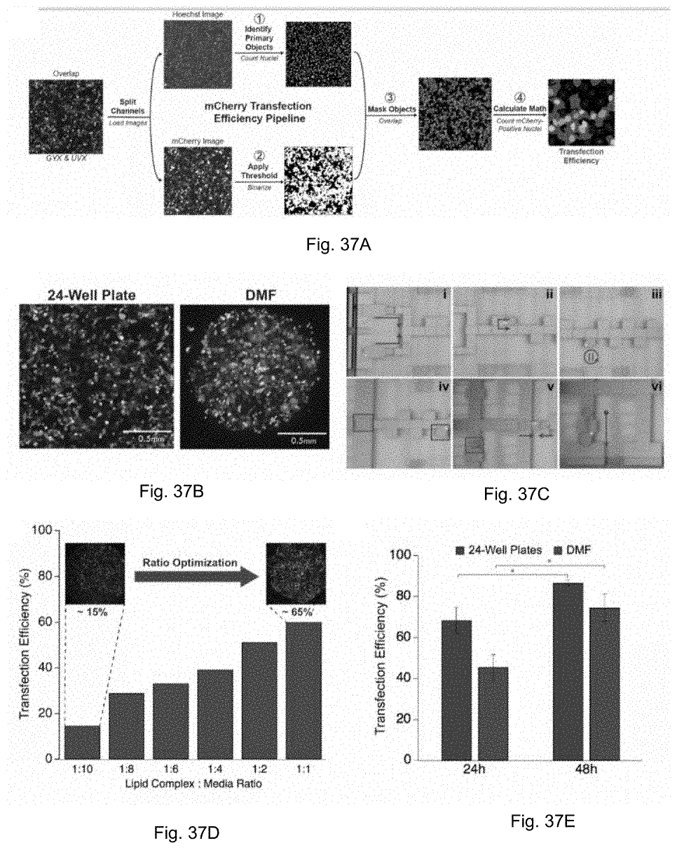

Optimizing gene-editing-transfection and knock-out. One of the advantages of digital microfluidics is its compatibility with external equipment and amenability with microscopy techniques for cellular analysis. In this example, microscopic imaging is used to analyze transfection and gene knockout of lung cancer cells on a DMF platform. Fluorescence-based imaging is performed by staining with fluorescent dyes or by the integration of fluorescent proteins and the use of reporter genes (e.g., mCherry, GFP) which can also help reveal information about cell state, phenotype and possibly provide some valuable insight on gene expression. As shown in FIG. 37A, two images (using UV and mCherry filters) displaying fluorescently labelled cells are counted, thresholded, and overlapped to measure the transfection efficiency. The simplicity of positioning the top plate on the bottom (such that the top plate was adjacent to the objective) is unique to digital microfluidics since there is no requirement of moving parts or tubing that may interfere with the imaging. FIG. 37B shows a representative image that displays two overlapped fluorescent-labelled images grown on the hydrophilic spot on DMF devices and for comparison, an overlapped image showing lung cancer cells grown on standard 24 well-plates. As shown, the morphologies of the cultured cells were similar on both surfaces.

For gene-editing assays, transfection may be used for the successful delivery of sgRNA and Cas9 into cells to produce double-stranded breaks at the target DNA. Lipid-mediated transfection remains popular due to the ease of use and its availability of reagents on the market and may be less harmful than electroporation techniques. One of the factors that affects cationic lipid-mediated transfection is the bioavailability of lipids assembled with the anionic nucleic acids or to the negatively supercharged proteins, which can be effectively directed to and engulfed by a large proportion of target cells. Concentration of lipid reagents and of nucleic acids can be used to maximize transfection efficiency while minimizing cytotoxicity. Seeking validation of the platform for the transfection of nucleic acids, the lipid-DNA complexes were generated by encapsulating an mCherry plasmid and delivering it to the cells on-chip to optimize transfection and measure the delivery efficiency.

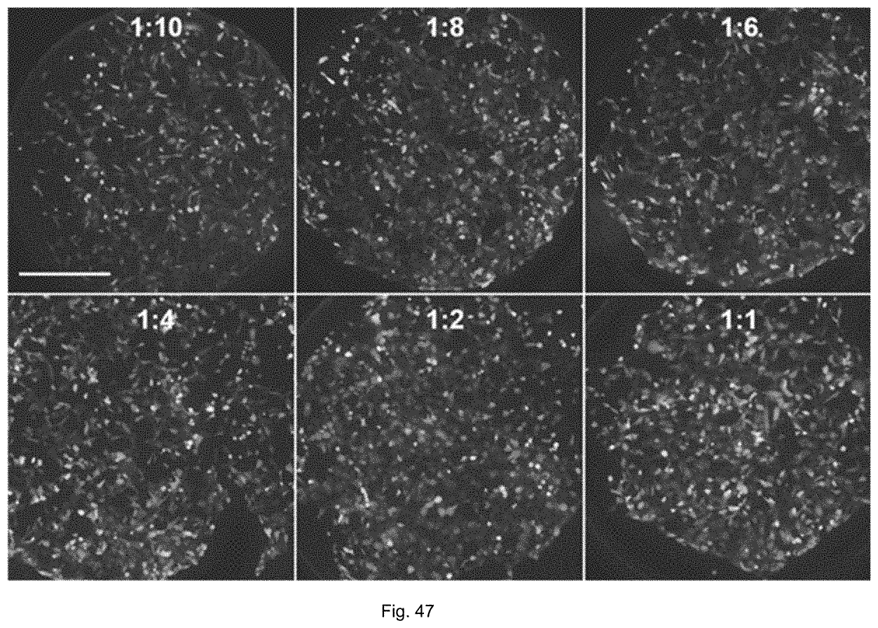

A portion of the experiment is depicted in FIG. 37C. Briefly, droplets of diluted lipids and DNA are dispensed, merged, mixed, and incubated. The droplet of complexed DNA-lipids is split and one droplet is used for passive dispensing to transfect the cells while the other droplet is used for further dilutions on the chip. The dilutions of lipid complexes in media were varied from 1:1 to 1:10 and it was determined that transfection efficiency is highest (˜65%) when a ratio of 1:1 is delivered to the cells on chip. Off-chip manufacturer's protocols suggest 1:10 ratios as the optimal, however, low efficiencies (˜15%) are observed when this ratio is performed on chip (FIG. 37D). Higher ratios (>1:10) were additionally conducted in well-plates, but it was observed that this ratio exhibited cytotoxic effects. Thus, other ratios may also be more optimal. It is hypothesized that signs of deterioration may be due to the presence of larger quantities of lipids which may cause toxicity to the cells due to the increase in likelihood in forming higher charge ratio complexes. While on the device in this example, higher ratios can be used since the lower volumes and cell densities require higher lipid complexes to media ratios for transfection to occur. As shown from FIG. 37D (inset images) and FIG. 47, the morphology of the cells at the 1:1 ratio is very similar to the 1:10 (and the other ratios) on device and do not show any signs of cell detachment or toxicity.

Next, with the selected ratios for each platform (1:10 in well plates; 1:1 on device), the transfection efficiency 24 to 48 h post-transfection was assessed. As shown in FIG. 37E, plasmids encoding mCherry to H1299 cells were successfully delivered using the device with transfection efficiencies that were highest after 48 h exhibiting ˜74.7%+/−6.8 compared to ˜45.7%+/−5.9 after 24 h (P<0.05). On-chip with well-plate techniques were also compared and it was observed no significant differences (P>0.05) in their efficiencies suggesting that DMF is a suitable alternative platform for transfection.

To test the efficacy of the ACE platform of achieving knockout of endogenous gene targets, H1299 cells that stably express enhanced GFP (eGFP) at the AAVS1 harboring sites were used, where there are no known adverse effects on cells resulting from the inserted DNA fragment. This allows simple phenotypic readouts of gene knock-out using GFP fluorescence to monitor the success of the platform in producing CRISPR-mediated genome editing.

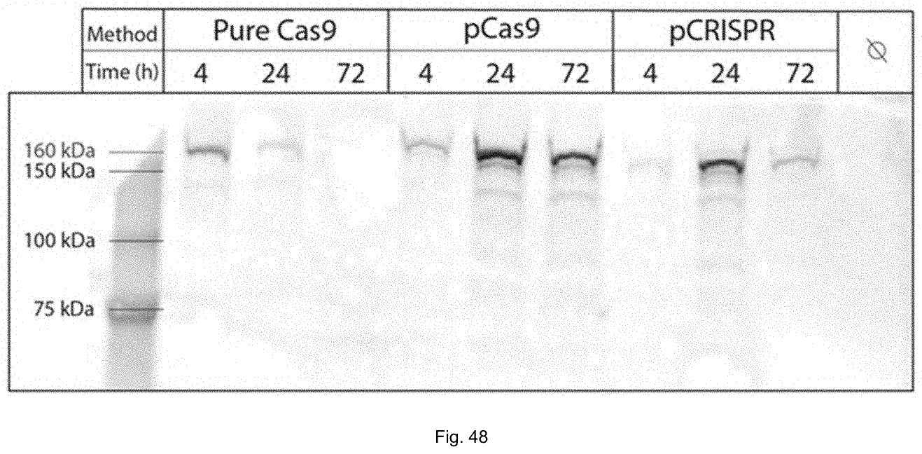

Initially, three experiments were performed to test the starting material for transfecting Cas9: (1) directly transfecting the Cas9 protein, (2) co-transfecting plasmids encoding Cas9 only and sgRNAs targeting GFP, and (3) transfecting an all-in-one pCRISPR plasmid containing both the Cas9 and sgRNA. As shown in FIG. 48, transfecting the all-in-one pCRISPR plasmid allowed high levels of Cas9 expression in 24 h while protein transfection showed lower levels at 24 h. In the Cas9 protein transfected cells, the level of Cas9 protein peaked at the first measured time point 4 h, then rapidly decreased and is barely detectable in the blot after 24 h. Upon realizing favorable expression patterns of the all-in-one pCRISPR plasmid, this format was opted for three reasons: (1) plasmid DNA is more stable as opposed to RNA and protein, (2) there is generally higher success for transfecting cells with one plasmid that can co-express both the sgRNA and the Cas9 protein as opposed to co-transfection, and (3) the ease by which such plasmids are redesigned (FIGS. 42 and 43).

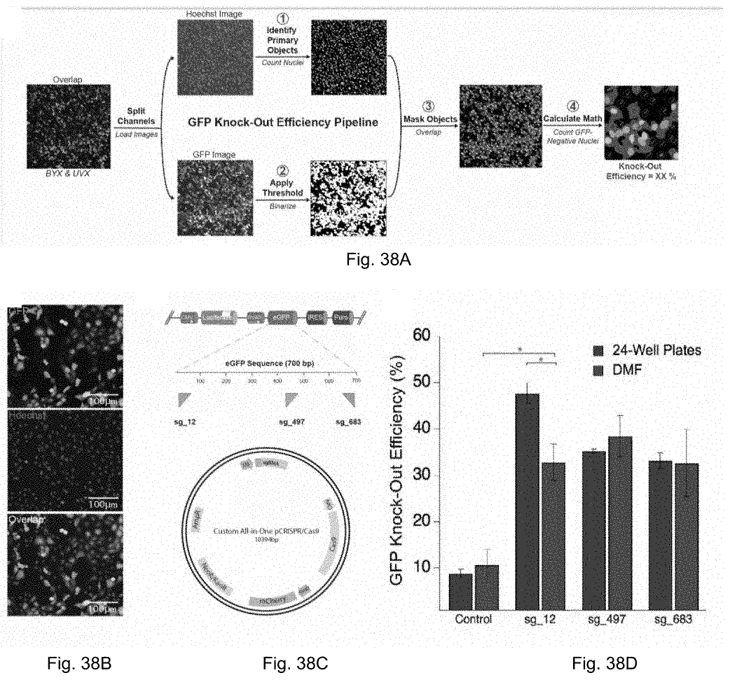

For proof-of-concept knock-out experiments, the eGFP was targeted and the knockout was analyzed using a pipeline similar to the transfection pipeline (FIG. 38A). Briefly, a Hoechst stained image and a GFP image (FIG. 38B) are processed by identifying nuclei and thresholding GFP regions-overlapping these images will highlight all the nuclei that are not overlapping GFP-positive regions, thereby being counted as cells exhibiting GFP knock-out. Comparing the number of knock-out nuclei to the total number of nuclei allows for a calculation of GFP knock-out efficiency. Three pCRISPR plasmids that contain an sgRNA targeting different loci in the GFP were designed and assembled: upstream (sg_12), middle (sg_497), downstream (sg_683) where the number represents the location of the base pairs for targeting (FIG. 38C).

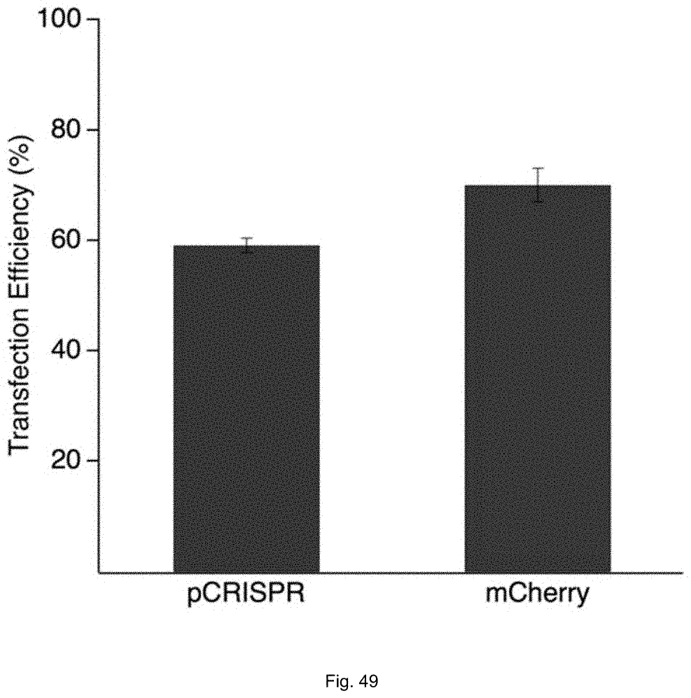

Cells were transfected with a larger pCRISPR plasmid (˜10.5 kb), with a reported transfection efficiency similar to a ˜5 kb mCherry plasmid (˜60% vs. 70%, as seen in FIG. 49) and knockout is observed on day 6. As shown in FIG. 38D, it was observed an average efficiency of ˜35% on-chip which is comparable to the well-plate experiments ˜39% (P>0.05). By analyzing the three different loci, it is observed that the knockout efficiencies for the middle and downstream loci using both technologies are very similar. However, it was observed a difference between the upstream loci knockout efficiencies (32.8% vs 47.7%). It is hypothesized that this variation is due to the use of well-plates for cell culturing in which adding medium (or any reagent) to the wells can result in uneven distribution, attachment, and growth of cells. This can cause a high variation in counting the cells using the pipeline especially after knockout. However, it is observed that there are no differences in the loci (32.8% for sg_12, 38.5% for sg_497, and 32.6% for sg_683) when using DMF and this it is believed is attributed to the homogeneity and reproducibility of cell culturing on device. Therefore, this particular example demonstrates the compatibility of DMF for knockout assays related to gene editing.

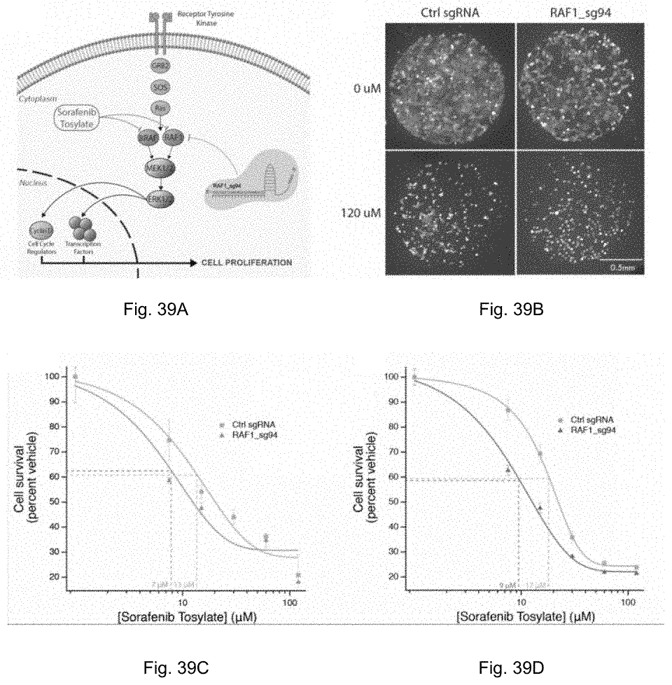

Evaluating MAPK/ERK pathway. To evaluate the potential of using the platform for gene editing, the relationship between gene function and cell phenotype was explored by studying a cellular signaling pathway. Cellular signaling is an intricate process driving various cellular activities such as protein synthesis, cell growth and cell senescence, which hold major implications regarding the understanding of tumor cell behavior and progression. Specifically, the MAPK/ERK (or also known as RAS-RAF-MEK-ERK) pathway is a highly conserved signaling cascade that plays a crucial role regulating cell fate decisions and is often upregulated in human cancers. The pathway is depicted in FIG. 39A, where a tyrosine receptor kinase serves to relay extracellular signaling to individual cells, through mitogen-activation. RAS and RAF genes are upstream components of the MAPK/ERK kinase signaling cascade, and therefore are a nodal point in cell proliferation, flagging them as potent oncogenes and natural targets for therapy. Generally, the RAS protein kinase gets phosphorylated and activated and the resulting RAS-GTP will complex with RAF in the plasma membrane. The order of subsequent events is still largely unknown, but a series of phosphorylation and dephosphorylation allow the dimerization of Raf protein kinases, used for the catalytic activation of RAF. Once activated, RAF kinases activate various effector proteins which govern cell proliferation. RAF proteins have been studied for characterization of human cancer—notably RAF1 (also known as c-RAF) was the first isoform to be identified as an oncogene, but interestingly mutations of RAF1 are rare in human cancers. Uncertainties surrounding the precise role of RAF1 have driven the interest in studying the effects of disrupting its encoding gene. This was initiated by regulating RAF1 protein expression at both the gene level by CRISPR-mediated knock-out and at the protein level by enzyme inhibition using protein inhibitor sorafenib tosylate.

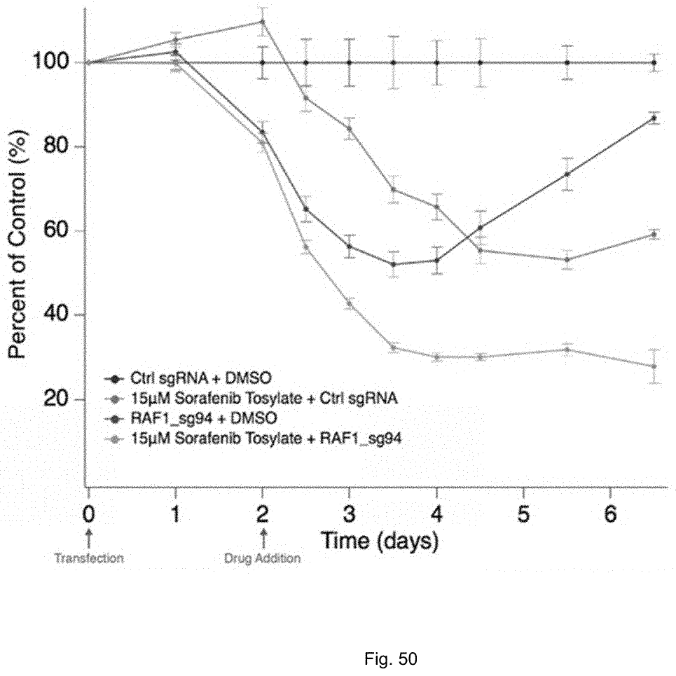

To assess the coupled effects of genome editing and drug inhibition, the H1299 cells with a pCRISPR targeting RAF1 or a control sgRNA were transfected and 15 micromolar sorafenib tosylate was added on day 2. Cells with RAF1 gene editing showed a minimum viability of ˜50% on day 4 over a 7-day experiment (FIG. 50). However, after day 4, cell viability levels started to increase while cells interrogated with both pCRISPR and sorafenib maintained low basal viability levels (˜25%) after day 4. This may be due to the heterogeneity of the cell population after transfection and knock-out or off-target effects caused by the single guide RNA. Evolving the Cas9 enzyme to be more versatile or using other types of RNA-guided endonucleases can perhaps alleviate these lower basal levels and efficiencies.

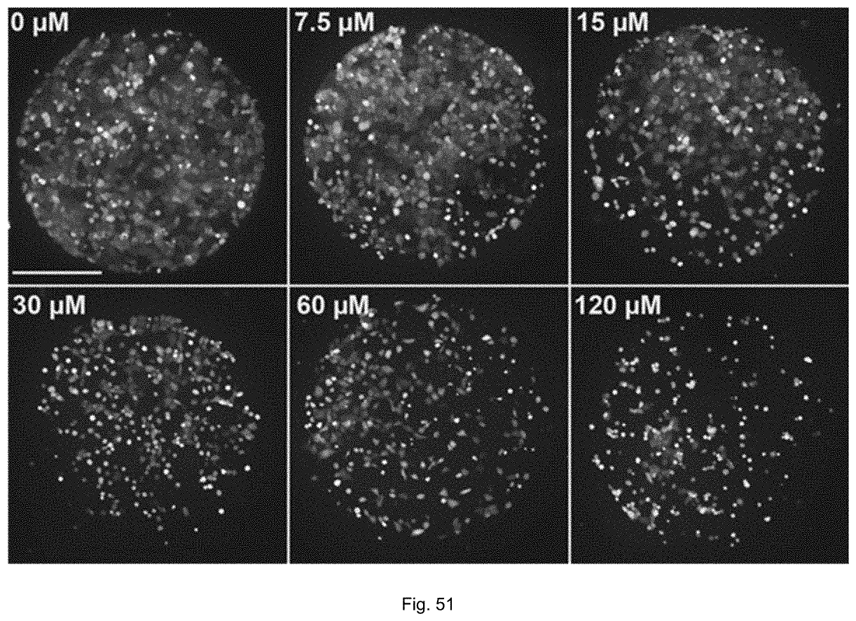

To verify the effects of targeting RAF1 by genome editing and enzymatic inhibition, H1299 cells were cultured, edited, assayed, and analyzed on the ACE platform following procedures for measuring transfection and knockout efficiencies. Images of the lung cancer cells that are transfected with and without pCRISPR targeting RAF1 and treated with the sorafenib inhibitor are analysed using the standardized imaging pipeline (FIG. 39B, FIG. 51). FIG. 39C (using ACE) shows a dose-response curve for sorafenib tosylate, illustrating the cell viability of the edited H1299 cells. The effects of RAF protein kinase inhibitor sorafenib tosylate with and without CRISPR-mediated RAF1 targeting were examined. For the case with CRISPR-mediated RAF1 targeting, the edited H1299 cells showed sensitivity in the linear micromolar range (˜7-35 micromolar) upon treatment of sorafenib.

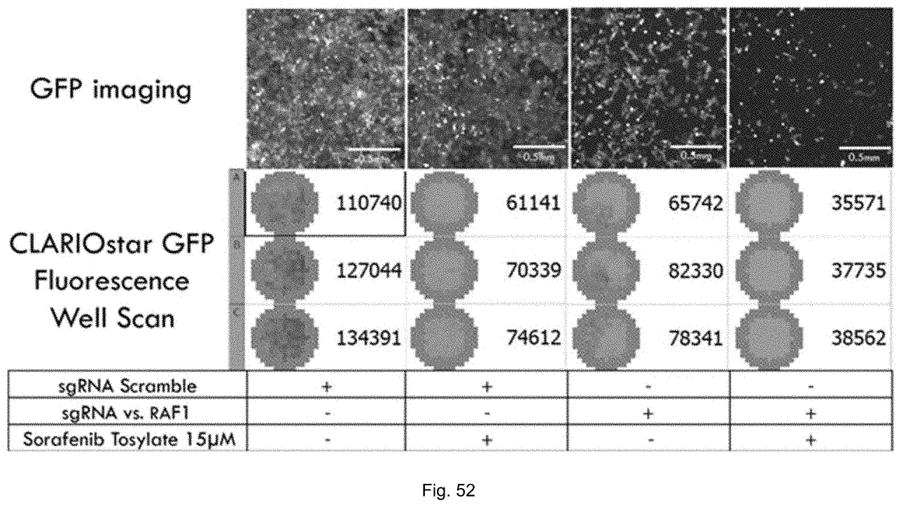

In addition, the viability of cells decreased compared to the control. Specifically, the fitted dose-response curve based on the sigmoid equation revealed that the inhibitory sorafenib concentration achieved half-maximal viability level (IC₅₀) is at 7.54 micromolar for the control while there is a ˜1.8-fold reduction (13.2 micromolar) when using pCRISPR targeting RAF1. An F-test revealed a significant difference between these two curves for concentrations in the linear regions of the curve (2.5-50 micromolar) (P<0.05). These on-chip results demonstrate that the addition of the single guide RNA targeting RAF1 shows a lower dose level to reduce cell viability. These results are also verified using well-plates and similar results were observed through fluorescence well-plate measurements and microscopy images (FIG. 39D; see examples of raw data in FIG. 52). Moreover, this is the first demonstration of gene-editing on a DMF platform. The ability to edit genes in cancer cells and to detect a phenotypic response highlights the potential of the ACE platform to investigate other pathways using gene-editing techniques.

The first demonstration of automated gene editing using digital microfluidics with an application to decipher cancer genes is presented in this example. The integration of gene-editing with DMF was characterized in terms of transfection and knockout efficiencies. A new standardized imaging pipeline was developed for the first time to analyze transfected and knockout cells. A gene-editing assay that targets the RAF1 gene in the MAPK/ERK pathway was performed to demonstrate the functionality in DMF-cultured lung cancer cells and to highlight a standardized imaging pipeline platform. The combination of automation, DMF, and gene-editing presented in this example can analyze a wide range of cancer genes.

Device Fabrication and Assembly.

Digital microfluidic devices were fabricated (FIG. 43). Briefly, designs were drawn using AutoCAD 2015 (Autodesk, San Rafael, Calif.) and photomasks were then printed in high-resolution (20,000 dpi) by CAD/Art Services Inc. (Bandon, Oreg.). The bottom plates bearing patterned electrodes were formed by standard photolithography techniques, in the Concordia Silicon Microfabrication Lab (ConSIM). Chromium substrates coated with photoresist were UV-exposed through the photomask (7 s, 42.4 mW/cm²) to imprint the transparency mask designs. Substrates were then developed in MF-321 positive photoresist developer (2 min, shaking), rinsed with DI water, dried under a stream of nitrogen and baked for 1 min at 115° C. The exposed chromium was then etched using CR-4 chromium etchant (3 min) and substrates were then rinsed with DI water and dried under a stream of nitrogen. Finally, devices were immersed in AZ300T photoresist stripper (3 min) to remove any remaining photoresist before being rinsed and dried under a stream of nitrogen.

Once the patterning step was completed, the substrates were immersed in a silane solution of deionized water, isopropanol and 3-(trimethoxysilyl)propyl-methacrylate (50:50:1) for dielectric priming during 15 min. Substrates were rinsed with isopropanol, DI water and then dried under a stream of nitrogen. Prior to the addition of the polymer coatings to complete the process, thermal tape was added on top of the contact pads to facilitate later removal of the polymer coatings from the contact pads and allow electrical contact for droplet actuation.

Parylene-C was used as a dielectric which was deposited by chemical vapor deposition in a SCS Labcoter 2 PDS 2010 (Specialty Coating Systems, Indianapolis, Ind.) achieving a homogenous final thickness of 7 micrometers. FluoroPel PFC1601V was used as a hydrophobic coating and was spin-coated in a Laurell spin-coater at 1500 rpm for 30 s followed by post-baking on a hotplate (180° C., 10 min).

The DMF top-plates were formed from a continuous ground electrode formed from an indium tin oxide (ITO) coated glass substrate. For typical ground plates, ITOs were spin-coated with the FluoroPel PFC1601V using the same program as described in the bottom-plate fabrication procedure. ITOs bearing an array of hydrophilic spots (e.g., circular regions of exposed ITO) for on-chip tissue culture were microfabricated using a fluorocarbon-liftoff procedure. ITOs were cleaned by immersion in an RCA solution comprising DI water, 28% aqueous ammonium hydroxide, 30% hydrogen peroxide (5:1:1 v/v/v) for 30 min at 80° C. on a hotplate. After rinsing, drying and dehydrating (2 min at 95° C.), the substrates were spin-coated with Shipley S1811 photoresist (10 s, 500 rpm, ACL=100 rpm and 60 s, 3000 rpm, ACL=500 rpm) and baked at 95° C. for 2 min. Slides were cut to the desired size (50×75 mm) using a Cuter's Mate (Creator's Stained Glass, Victoria, BC) and vented under a stream of nitrogen. Substrates were exposed through the photomask with an array of six 1.75 mm diameter circular features (10 s, 42.4 mW/cm²) and developed in MF-321 (3 min). After rinsing, air-drying and dehydrating (1 min, 95° C.), top-plates were then flood exposed (10 sec, 42.4 mW/cm²), spin-coated with 1% Teflon in FC-40 (10 s, 500 rpm, ACL=100 rpm and 60 s, 3000 rpm, ACL=500), and post-baked on a hotplate (165° C., 10 min). After allowing to cool on aluminum foil for 2 min, substrates were immersed in acetone with gentle agitation for 10-15 s until the Teflon-AF over the patterned sites was lifted off. After being rinsed with DI water and dried under a stream of nitrogen, droplets of AZ300T stripper was gently placed over the spots and substrates were placed aside for 1 min followed by rinsing with DI water and air-drying. Post-baking followed to reflow the Teflon-AF at 165° C., 210° C., and 300° C. for 5 min at each temperature.

Complete devices were assembled with the continuous ground ITO top-plate and the chromium electrode-bearing bottom plate, being joined by stacking two layers of double sided tape to a gap height of approximately 140 micrometers. Alignment of the ITO top plate above the bottom plate was performed with care such that the edge of the top plate was adjacent to the outer-edges of the reservoir electrodes of the bottom-plate pattern (see FIG. 34). Moreover, each 25 mm×75 mm top plate was roughly aligned to the electrodes over which the virtual microwells were required.

Automation setup and device operation. The automation system (FIG. 44) used a MATLAB (Natlick, Mass.) program that is used to control an Arduino Uno microcontroller (Adafruit, N.Y., USA). Driving input potentials of 130-270 V_{RMS} were generated by amplification of a sine wave output from a function generator (Agilent Technologies, Santa Clara, Calif.) operating at 10 kHz by a PZD-700A amplifier, (Trek Inc., Lockport, N.Y.) and delivered to the PCB control board. The Arduino controls the state of high-voltage relays (AQW216 Panasonic, Digikey, Winnipeg, MB) that are soldered onto the PCB control board. The logic state of an individual solid-state switch is controlled through an I²C communication protocol by an I/O expander (Maxim 7300, Digikey, Winnipeg, MB). This control board is mated to a pogo pin interface (104 pins), where each switch delivers a high-voltage potential (or ground) signal to a contact pad on the DMF device. The GitHub registry has instructions to assemble the hardware and to install the open-source software program to execute the automation system.

To start gene-editing experiments, reagent loading was achieved by pipetting a droplet of liquid onto the outer-edge of a reservoir electrode and adjacent to the gap between the bottom and top plates and actuating the reservoir electrode. Once inside the reservoirs, the droplets were then actively dispensed, moved, mixed, or merged by sequential actuation of neighboring electrodes on the bottom plates. Active dispensing was achieved over three electrodes and results in a droplet with a diameter of the same size as the electrodes (e.g., a unit droplet). To initiate passive dispensing, it was achieved by moving an actively dispensed droplet over the vacant lift-off spot. At times, contents on this spot may be displaced with the contents of a new source droplet. Generally, all droplets containing proteins were supplemented with 0.05% Pluronics F-68. Waste and unused fluids were removed by delivering them to reservoirs and removed using KimWipes (Kimberly-Clark, Irving, Tex.).

TABLE 11

| Cells | Genotype | Source |
|---|---|---|
| E. coli DH5α | fhuA2 Δ(argF-lacZ)U169 | V. Martin |
| | phoA glnV44 Φ80 | |
| | Δ(lacz)M15 gyrA96 recA1 | |
| | relA1 endA1 thi-1 hsdR17 | |

| Cell Line | Transgene Integration | Source |
|---|---|---|
| NCI-H1299 (Human lung | KanR | Genecopoeia |
| squamous cell carcinoma | | SL001 |
| dual-labeled stable) | | |

| Plasmids | Relevant characteristics | Addgene # |
|---|---|---|
| mCherry2-N1 | KanR | 54517 |
| All_in_one_CRISPR/ | AmpR | 74293 |
| Cas9_LacZ | | |
| pSpCas9(BB)-2A- | AmpR, PuroR | 62988 |
| Puro (PX459) v.2.0 | | |

TABLE 12

| Custom pCRISPR Plasmids | Custom Sequence | PAM | Source |
|---|---|---|---|
| pCRISPR_eGFP_191 | −/ACTGCACGCCGTAGGTCAGGG | TGG | This study |
| (SEQ ID NO: 13) | | | |
| pCRISPR_eGFP_314 | +/GCAACTACAAGACCCGCGCCG | AGG | This study |
| (SEQ ID NO: 14) | | | |
| pCRISPR_eGFP_369 | +/TCGATGCCCTTCAGCTCGATG | CGG | This study |
| (SEQ ID NO: 15) | | | |
| pCRISPR_eGFP_497 | +/TCAAGATCCGCCACAACATCG | AGG | This study |
| (SEQ ID NO: 16) | | | |
| pCRISPR_eGFP_683 | −/CCATGCCGAGAGTGATCCCGG | CGG | This study |
| (SEQ ID NO: 17) | | | |
| pCRISPR_RAFI_94 | +/GCCGCCCGAGAGTCTTAATCG | CGG | This study |
| (SEQ ID NO: 18) | | | |
| PX459_eGFP_12-31 | +/GGGCGAGGAGCTGTTCACCG | GGG | Genscript |
| (SEQ ID NO: 19) | | | |

TABLE 13

| Gene | Orientation | Sequence |
|---|---|---|
| g-block_universal | Forward | ATATATCGTCTCGAATTGAAAGTATTTCGATTTCTTGGGT |
| | | (SEQ ID NO: 20) |
| g-block_universal | Reverse | ATAATTCGTCTCTAGCGCAAAACGCCTAACCCTAAGCAGA |
| | | TTCTTCATGCAATTGTGTCTAGAAAAAAGCACCGACTCGG |
| | | TG (SEQ ID NO: 21) |
| SP6 sequencing primers | Forward | ATTTAGGTGACACTATAG (SEQ ID NO: 22) |

FIGS. 34, 35, and 36 illustrate digital microfluidic automated gene-editing assays used in this example. FIG. 34 is a top-view schematic of a digital microfluidic device used for cell culturing, transfection, gene-editing, and analysis. FIG. 35 is a side-view schematic showing adherent cells culture on the top-plate. The cells are transfected using lipid-mediated delivery of plasmids and then measured for knockout by imaging techniques. FIG. 35 shows a step-by-step CRISPR-Cas9 knock-out process at the cellular level This figure includes: (1) assembly of DNA-lipid complex, (2) endocytosis, (3) endosomal escape, (4) transduction of Cas9 and sgRNA, (5) translation of Cas9 mRNA, (6) Cas9 ribonucleoprotein assembly, (7) nuclear localization, (8) double-strand break, and (9) DNA repair by non-homologous end joining and subsequent genomic disruption by indels. FIG. 36 is a timeline showing the process of automated gene-editing on chip.

FIGS. 37A, 37B, 37C, 37D, and 37E show lipid-mediated transfection experiments in this example. FIG. 37A is a schematic showing the imaging pipeline used for analyzing transfection. FIG. 37B shows microscopy images of mCherry-transfected NCI-H1299 cells in the well-plate format and on DMF devices. FIG. 37C shows a video sequence depicting the mixing of lipids and DNA and the passive dispensing procedure onto the hydrophilic spot. Frame (i) shows dispensing of droplets containing DNA and lipids from separate reservoirs and merging both unit droplets. Frame (ii) displays mixing of DNA and lipids on a 2×2 electrode array. Frame (iii) shows incubation of complexes for 10 min. Frame (iv) shows the preparation of the dilution by dispensing a droplet of liquid media. Frame (v) show the 1:1 dilution of lipid complexes in media. Frame (vi) shows the passive dispensing of dilute lipids onto the cell culture spot. FIG. 37D is a plot showing the optimization of the lipid complex to media ratio for transfection on device. FIG. 37E is a plot of the transfection efficiency for a mCherry plasmid in the well-plate and on DMF devices. All plots show error bars with +/−1 s.d, n=3 and *P<0.05.

FIGS. 38A, 38B, 38C, and 38D show a knockout of stably integrated eGFP. FIG. 38A shows a schematic showing the imaging pipeline used for analyzing knockout. FIG. 38B shows an image set (Hoechst, GFP, overlap) processed by CellProfiler to assess eGFP knock-out efficiency. FIG. 38C shows a plasmid map of the pCRISPR plasmid used showing the transgene integration in NCI-H1299 and sgRNA target regions of eGFP. FIG. 38D show a plot shown for the knockout of GFP in well-plates compared to the microscale. Error bars are +/−1 s.d. with n=3 and *P<0.05.

FIGS. 39A, 39B, 39C, and 39D show identification of cancer genes in the MAPK/ERK pathway. FIG. 39A is a cartoon illustrating signal transduction in the Ras pathway that leads to eventual cell proliferation. The targeted genes using sgRNAs and the added drug (sorafenib) are indicated on the diagram. FIG. 39B shows microscopy images of the H1299 cells with sorafenib inhibitor (0 and 120 micromolar in DMSO) and with guide targeting RAF1 and eGFP (control).

FIG. 39C shows on-chip and FIG. 39D shows off-chip dose-response curve for H1299 cells transfected with and without individual guides targeting Raf-1 at different concentrations of sorafenib. Referring to FIG. 40, the sgRNA sequence (SEQ ID NO: 2) represents the template designed for all sgRNAs. It has the U6 Promoter, the variable seed sequence, the dCas9 handle and the S. pyogenes terminator. The seed sequences varied according to the target region (see Tables 11 and 12). All eight constructs were synthesized by Integrated DNA Technologies, Inc. (Coralville, Iowa).

FIG. 41 shows a gel electrophoresis image of the PCR products of the synthesized CRISPR guides, yielding g-blocks. PCR products were loaded into a 0.8% agarose gel in TAE buffer and resolved at 130 V for 30 min. These represent the g-blocks flanked with BsmBI cut sites, ready for insertion into a pCRISPR backbone: (1) KRAS_5608; (2) KRAS_41162; (3) RAF1_94; (4) RAF1_253; (5) RAF1_64486; (6) EGFP_191; (7) EGFP_314; (8) EGFP_369; (9) EGFP_497; (10) EGFP_683.

FIG. 42 shows blue/white screening. A schematic showing the procedure of inserting a CRISPR guide into a Cas9 vector backbone. An all-in-one pCRISPR template tailored to blue-white screening was used. The LacZ-alpha open reading frame, necessary to complement Δ(lacZ)M15 (delta) for functional beta-galactosidase expression, was inserted between two BsmBI flanking sites. One-pot assembly reactions containing the all-in-one pCRISPR template, the restriction enzymes, the g-block and the T4 DNA ligase were placed in a thermal cycler and the product was transformed into E. coli. Cells were plated on LB Agar with S-Gal, a colorless substrate that gets hydrolyzed by beta-galactosidase and results in blue bacterial colonies. Cells that were transformed with recombinant vectors of interest would be white, and those transformed with non-recombinant vectors would be blue.

FIG. 43 shows a schematic of DMF device and top-plate fabrication. Bottom-plate fabrication followed a photolithography procedure (left) and top-plate fabrication followed a standard lift-off procedure (right).

FIG. 44 shows a microfluidic automation system for gene-editing used in this example. The automation system includes a custom MATLAB program interfaced to an Arduino Uno microcontroller. The Arduino controls the state of high-voltage relays on a switching control board. Sine waves are generated from a function generator operating at 10 kHz and amplified using a high-voltage amplifier, producing driving input potentials of 130-270 V_{RMS} to the control board. The control of the state of an individual switch is done through an I²C communication protocol using an I/O expander. The control board is mated to a pogo pin board, where each switch is wired to an individual pogo-pin, in contact with a contact pad. The device is imaged live through a CMOS camera.

FIGS. 45A and 45B shows a 3D-printed humidified chamber and microscope holder for imaging. FIG. 45A shows a cell humidified chamber with cover to prevent evaporation of droplets. The design includes a rack above a water reservoir, on which the devices are placed and of a lid to prevent evaporation and allow saturation in humidity. FIG. 45B shows a microscope holder tailored to digital microfluidic devices, with opaque cover for fluorescence microscopy.

FIGS. 46A and 46B show optimization of chip configuration and electrode design in this example. In FIG. 46A, the first design shows a configuration with square electrodes. In FIG. 46B, the current design is modified to have interdigitated electrodes to facilitate droplet movement.

FIG. 47 shows optimization of on-chip transfection using various dilutions of lipid complexes in liquid media. Overlapped eGFP and mCherry images show empirical transfection efficiencies for a range of different ratios (1:10, 1:8, 1:6, 1:4, 1:2, and 1:1). The 1:1 ratio shows highest transfection efficiency. Scale bar=0.5 mm.

FIG. 48 is a Western blot showing Cas9 protein levels comparing different starting material of Cas9 into NCI-H1299 cells. Lipid-mediated transfection was done using three different starting materials (DNA and protein), and lysates were collected at three different time-points (4, 24, and 72 h). Lane (1) shows pure Cas9 protein to assess transfection of RNP complexes. Lane (2) shows Cas9 expressing plasmid, pCas9, to assess co-transfection of pCas9 with an sgRNA plasmid. Lane (3) shows transfection of pCRISPR all-in-one plasmid (Cas9 and sgRNA). A negative control was transfected with the mCherry2-N1 plasmid and the lysate was collected after 24 h. The expected protein size of Cas9 is 160 kDa.

FIG. 49 shows a plot of the transfection efficiency for both the All_in_one_CRISPR/Cas9_LacZ (pCRISPR) and mCherry2-N1. pCRISPR has a reporter mCherry gene under an SV40 promoter, and a CMV promoter was used for the mCherry plasmid. For transfection, a 1:10 ratio of lipid complexes to media was used. Images of the transfected H1299 cells were taken after 48 h and processed using the standardized transfection pipeline.

FIG. 50 shows a plot showing progression of cell viability over time. Four conditions were tested by acquiring fluorescent measurements over 7 days to assess proliferation. Cells were transfected on day 0 with an sgRNA targeting RAF1 or a scramble sgRNA. After 48 h post-transfection, a drug Sorafenib Tosylate or DMSO and was added to the guides. All readings were taken in triplicate and error bars represent +/−1 s.d.

FIG. 51 shows microscopy images of H1299 cells on-chip. Each image shows a condition that is treated with the enzymatic inhibitor sorafenib tosylate. The images are taken on day 5. Scale bar=0.5 mm.

FIG. 52 shows raw data showing the absolute fluorescence and the morphology of the H1299 cells. Four conditions were tested and microscopy fluorescent images were captured on day 5 using GFP filter set.

The embodiments of the above paragraphs are presented in such a manner in the present disclosure so as to demonstrate that every combination of embodiments, when applicable can be made. These embodiments have thus been presented in the description in a manner equivalent to making dependent claims for all the embodiments that depend upon any of the preceding claims (covering the previously presented embodiments), thereby demonstrating that they can be combined together in all possible manners.

In addition, the definitions and embodiments described in any of the sections above are intended to be applicable to other embodiments herein described for which they are suitable as would be understood by a person skilled in the art.

Thus, while several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including,” “having,” and their derivatives. Finally, terms of degree such as “substantially,” “about,” and “approximately” as used herein mean a reasonable amount of deviation of the modified term such that the end result is not significantly changed. These terms of degree should be construed as including a deviation of at least +/−10% of the modified term if this deviation would not negate the meaning of the word it modifies.

As used in this specification and the appended claims, the singular forms “a,” “an,” and “the” include plural references unless the content clearly dictates otherwise. Thus, for example, a composition containing “a compound” includes a mixture of two or more compounds. It should also be noted that the term “or” is generally employed in its sense including “and/or” unless the content clearly dictates otherwise.

All definitions, as defined and used herein, should be understood to control over dictionary definitions, definitions in documents incorporated by reference, and/or ordinary meanings of the defined terms.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1-88. (canceled)

89. A method of gene-editing a mammalian cell, the method comprising:
culturing mammalian cells within a digital microfluidics device; dispensing a droplet containing a transfection complex on the digital microfluidics device; and
exposing the mammalian cells to the droplet to transfect at least some of the mammalian cells using the transfection complex.

90. The method of claim 89, wherein the transfection complex is a lipid transfection complex.

91. The method of claim 89, further comprising:
dispensing a first droplet and a second droplet on the digital microfluidics device, the first droplet containing a first transfection reagent and the second droplet containing a second transfection reagent; and
merging the first and second droplets to form the droplet containing the transfection complex, wherein the first and second transfection reagents react to form the transfection complex.

92. (canceled)

93. The method of claim 91, wherein first transfection reagent comprises liposome-forming lipids.

94-104. (canceled)

105. The method of claim 91, wherein the second transfection reagent comprises a nucleic acid.

106. The method of claim 91, wherein the second transfection reagent comprises a plasmid.

107. The method of claim 106, wherein the plasmid comprises a gRNA sequence.

108-109. (canceled)

110. The method of claim 106, wherein the plasmid encodes Cas9.

111-114. (canceled)

115. The method of claim 91, wherein the second transfection reagent comprises a nucleic acid binding endonuclease.

116. The method of claim 91, wherein the second transfection reagent comprise a CRISPR-associated nuclease.

117. The method of claim 91, wherein the second transfection reagent comprises Cas9 protein.

118. (canceled)

119. The method of claim 91, wherein the second transfection reagent comprises an HDR template.

120-121. (canceled)

122. The method of claim 91, comprising merging the first droplet and the second droplet to form the droplet containing the transfection complex at a mixing region within the digital microfluidics device.

123-135. (canceled)

136. The method of claim 89, comprising culturing the mammalian cells within a cell culture region within the digital microfluidics device.

137-141. (canceled)

142. The method of claim 141, wherein culturing mammalian cells comprises:
seeding mammalian cells into a reservoir in the digital microfluidics device; and
dispersing the mammalian cells from the reservoir to the cell culture region.

143-150. (canceled)

151. The method of claim 141, further comprising applying a removal agent to remove adherent mammalian cells from the cell culture region.

152-154. (canceled)

155. The method of claim 89, comprising supplying reagent to the mammalian cells from a media feed region within the digital microfluidics device.

156-158. (canceled)

159. The method of claim 89, further comprising determining transfection of the mammalian cells after exposing the mammalian cells to the droplet.

160-162. (canceled)

163. The method of claim 159, wherein determining transfection of the mammalian cells comprises moving the mammalian cells to an assay site within the digital microfluidics device.

164-171. (canceled)

172. A method, comprising:
culturing mammalian cells within a digital microfluidics device;
transfecting the mammalian cells within the digital microfluidics device; and
determining transfection of the mammalian cells within the digital microfluidics device.

173. The method of claim 172, comprising transfecting the mammalian cells using a CRISPR-associated nuclease.

174. (canceled)

175. The method of claim 172, comprising transfecting the mammalian cells using electroporation.

176. (canceled)

177. The method of claim 172, comprising transfecting the mammalian cells using chemical transfection.

178-179. (canceled)

180. The method of claim 172, further comprising:
dispensing a first droplet and a second droplet on the digital microfluidics device, the first droplet containing a first transfection reagent and the second droplet containing a second transfection reagent; and
merging the first and second droplets to form the droplet containing the transfection complex, wherein the first and second transfection reagents react to form the transfection complex.

181-182. (canceled)

183. The method of claim 172, comprising culturing the mammalian cells within a cell culture region within the digital microfluidics device.

184. The method of claim 172, comprising culturing the mammalian cells within an incubator containing the digital microfluidics device.

185. The method of claim 172, wherein the mammalian cells are present within a cell culture region within the digital microfluidics device.

186. The method of claim 185, wherein the mammalian cells are adhered to a cell culture region within the digital microfluidics device.

187-192. (canceled)
